(19) United States
(12) Patent Application Publication
(10) Pub. No.: US 2026/0285943 A1
(43) Pub. Date: Sep. 24, 2026

(54) POLYMERIZED HEMOGLOBINS AND RELATED COMPOUNDS AND METHODS

(71) Applicant: The Regents of the University of California, Oakland, CA (US)

(72) Inventors: Pedro Cabrales, La Mesa, CA (US); Carlos Munoz, San Diego, CA (US); Daniela Lucas, La Jolla, CA (US)

(21) Appl. No.: 19/475,989

(22) PCT Filed: Apr. 17, 2024
(86) PCT No.: PCT/US2024/025049

§ 371 Date: Oct. 16, 2025

Related U.S. Application Data
(60) Provisional application No. 63/459,941, filed on Apr. 17, 2023.
(60) Provisional application No. 63/535,934, filed on Aug. 31, 2023.

(51) Int. Cl. C07K 14/805; C12N 9/10

(52) U.S. Cl. CPC C07K 14/805; C12N 9/1044

(57) ABSTRACT

The present disclosure is related to a solution comprising hemoglobins including polymerized hemoglobins (TgHbs). In some embodiments, a method can comprise adding a transglutaminase to a solution including hemoglobins, to polymerize at least some of the hemoglobins to form polymerized hemoglobins.

### CROSS-REFERENCE TO RELATED APPLICATIONS

This application claims the benefit of U.S. Provisional Appl. No. 63/459,941, filed Apr. 17, 2023; and U.S. Provisional Appl. No. 63/535,934, filed Aug. 31, 2023, each of which is incorporated by reference as if fully set forth herein.

### FIELD

This Application relates to methods for blood engineering, and particularly polymerized hemoglobins.

### BACKGROUND

Blood is a vital fluid that circulates throughout the body, carrying oxygen and nutrients to the cells and removing waste products. It is a major component of the immune system. There have been a variety of medical technology involving blood. For example, blood transfusions are used to replace blood that has been lost due to injury, surgery, or disease. Blood transfusions can also be used to treat anemia, a condition in which the body does not have enough red blood cells. Blood products are made from donated blood and are used to treat a variety of medical conditions. Some examples of blood products include plasma, platelets, and red blood cells. Blood is a valuable resource that is essential for many medical treatments. Advances in blood technology are helping to improve the lives of patients.

### SUMMARY

This Summary is provided to introduce blood engineering, and particularly for artificial blood that are further described below in the Detailed Description. This Summary is not intended to identify key aspects or essential aspects of the claimed subject matter.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

The present disclosure is related to a method comprising introducing a transglutaminase to a solution that contains a plurality of hemoglobins to polymerize at least a portion of the plurality of the hemoglobins to form polymerized hemoglobins.

In some embodiments, prior to introducing the transglutaminase, solid content of the plurality of hemoglobins in the solution can be from about 1 g/dL to about 25 g/dL.

In some embodiments, prior to introducing the transglutaminase, solid content of the plurality of hemoglobins in the solution can be from about 5 g/dL to about 20 g/dL.

In some embodiments, prior to introducing the transglutaminase, solid content of the plurality of hemoglobins in the solution can be from about 10 g/dL to about 15 g/dL.

In some embodiments, introducing the transglutaminase may comprise introducing the transglutaminase at a ratio of from about 2 to about 10 enzyme units of the transglutaminase per gram of the plurality of hemoglobins.

In some embodiments, introducing the transglutaminase may comprise introducing the transglutaminase at a ratio of from about 5 enzyme units of the transglutaminase per a gram of the plurality of hemoglobins.

In some embodiments, the transglutaminase comprises a calcium-independent transglutaminase.

The present disclosure is related to a composition comprising a plurality of hemoglobins, wherein at least a portion of the plurality of hemoglobins may comprise polymerized hemoglobins and an amount of transglutaminase.

In some embodiments, the amount of transglutaminase may be a trace amount.

In some embodiments, an aqueous solution may comprise the composition comprising a plurality of hemoglobins, wherein at least a portion of the plurality of hemoglobins may comprise polymerized hemoglobins and an amount of transglutaminase.

In some embodiments, solid content of the plurality of hemoglobins in the solution may be from about 1 g/dL to about 25 g/dL.

In some embodiments, solid content of the plurality of hemoglobins in the solution may be from about 5 g/dL to about 20 g/dL.

In some embodiments, solid content of the plurality of hemoglobins in the solution may be from about 10 g/dL to about 15 g/dL.

In some embodiments, solid content of the plurality of hemoglobins in the solution may be about 20 g/dL.

In some embodiments, the aqueous solution may exhibit a colloid osmotic pressure of less than about 50 mmHg.

In some embodiments, the aqueous solution may exhibit a colloid osmotic pressure of less than about 40 mmHg.

In some embodiments, the aqueous solution may exhibit a colloid osmotic pressure of from about 30 mmHg to about 35 mmHg.

In some embodiments, wherein solid content of the plurality of hemoglobins in the solution may be about 10 g/dL.

In some embodiments, when an oxygenation state of the polymerized hemoglobins is about a fully oxygenated state of the polymerized hemoglobins, a viscosity of the aqueous solution at a shear rate of about 100 s⁻¹ at about 37° C. can be from about 35 cP to about 50 cP.

In some embodiments, when the oxygenation state is about the fully oxygenated state of the polymerized hemoglobins, the viscosity of the aqueous solution at the shear rate of about 100 s⁻¹ at about 37° C. can be about 42 cP.

In some embodiments, a viscosity of the aqueous solution at a shear rate of about 100 s⁻¹ may change at least by about 25 cP or more when an oxygenation state of the polymerized hemoglobins changes from about a deoxygenated state to about a fully oxygenated state.

In some embodiments, the transglutaminase may comprise a calcium-independent transglutaminase.

The present disclosure is related to an aqueous solution comprising a plurality of hemoglobins, wherein at least a portion of the plurality of hemoglobins can comprise transglutaminase-polymerized hemoglobins (TgHbs), and wherein a viscosity of the aqueous solution at a shear rate of about 100 s⁻¹ at about 37° C. can change by about 25 cP or more when an oxygenation state of the TgHbs changes from about a deoxygenated state to about a fully oxygenated state.

In some embodiments, the viscosity of the aqueous solution at the shear rate of about 100 s⁻¹ at about 37° C. can change by about 30 cP when the oxygenation state of the TgHbs changes from about deoxygenated state to about fully oxygenated state.

In some embodiments, solid content of the plurality of hemoglobins in the solution can be from about 1 gram per g/dL to about 25 g/dL.

In some embodiments, solid content of the plurality of hemoglobins in the solution can be from about 5 gram per g/dL to about 20 g/dL.

In some embodiments, solid content of the plurality of hemoglobins in the solution can be from about 10 gram per g/dL to about 15 g/dL.

In some embodiments, solid content of the plurality of hemoglobins in the solution can be from about 10 gram per deci-Liter (g/dL) to about 20 g/dL.

In some embodiments, solid content of the plurality of hemoglobins in the solution can be about 20 g/dL.

In some embodiments, the aqueous solution may exhibit a colloid osmotic pressure of less than about 50 mmHg.

In some embodiments, the aqueous solution may exhibit a colloid osmotic pressure of less than about 40 mmHg.

In some embodiments, the aqueous solution may exhibit a colloid osmotic pressure of from about 30 mmHg to about 35 mmHg.

In some embodiments, solid content of the plurality of hemoglobins in the solution can be about 10 g/dL.

In some embodiments, when the oxygenation state is around the fully oxygenated state of the TgHbs, the viscosity of the aqueous solution at a shear rate of about 100 s⁻¹ at about 37° C. can be from about 35 cP to about 50 cP.

In some embodiments, when the oxygenation state is around the fully oxygenated state of the TgHbs, the viscosity at the shear rate of about 100 s⁻¹ at about 37° C. can be about 42 cP.

### BRIEF DESCRIPTION OF THE FIGURES

In this Application:

FIG. 1 describes the hypovolemic infusion protocol in Golden Syrian hamsters in some embodiments.

FIG. 2 indicates results mean arterial pressure and heart rate absolute values and relative to baseline following a hypovolemic infusion of hHb, HBOC-201, and TgHb in some embodiments.

FIG. 3 indicates results of log (shear rate) versus log (viscosity) of TgHb over a variety of shear rates starting from 0 s⁻¹ and up to 1000 s⁻¹ using a plate and cone geometry in some embodiments.

FIG. 4 indicate results of log (shear rate) versus log (shear stress) of TgHb over a variety of shear rates starting from 0 s⁻¹ and up to 1000 s⁻¹ using a plate and cone geometry in some embodiments.

FIG. 5 indicates results of Pharmacokinetics and oxidation rate of TgHb from 0 hours up to 48 hours taking measurements of plasma Hb and plasma metHb at 0, 2, 4, 8, 12, 24, and 48 hours in some embodiments.

FIG. 6 indicates results of microcirculatory arterioles' diameter and flow relative to baseline following a hypovolemic infusion of hHb, HBOC-201, or TgHb. The micro vessels were separated by diameter ≤40 μm, 40<x≤60 μm, or <60 μm. † is significant to baseline in some embodiments.

FIG. 7 indicates results of microcirculatory venules' diameter and flow relative to baseline following a hypovolemic infusion of hHb, HBOC-201, or TgHb. The micro vessels were separated by diameter ≤40 μm, 40<x≤60 μm, or <60 μm. † is significant to baseline in some embodiments.

FIG. 8 indicates results of functional capillary density relative to baseline following the hypovolemic infusion of hHb, HBOC-201, or THb. † is significant to baseline in some embodiments.

FIG. 9 indicates levels of cardiac CRP, cardiac IL-6, cardiac IL-1, cardiac TNF-α, cardiac MCP-1 and cardiac Troponin proteins that can indicate one or more status of the cardiac function and cardiac inflammation in hamster in some embodiments.

FIG. 10 indicates levels of plasma BUN, urine NGAL and urine creatinine, which can be based on to speculate one or more kidney functions in some embodiments.

FIG. 11 indicates concentrations of renal IL-6, renal IL-1, CXCL1 and IL-10, which can be based on to speculate one or more kidney inflammation in hamsters in some embodiments.

FIG. 12 indicates Hepatic CXCL1, plasma AST and plasma ALT concentrations, which can be based on to speculate one or more liver functions in hamsters in some embodiments.

FIG. 13 indicates concentrations of plasma IL-6, plasma CXCL1, and plasma IL-10, which can be evaluated to speculate endothelium health in hamsters in some embodiments.

FIG. 14 indicates concentrations of ferritin and bilirubin in hamsters in some embodiments.

FIG. 15 indicates concentrations of plasma epinephrine and plasma norepinephrine which can be evaluated to speculate nervous system health in hamsters in some embodiments.

FIG. 16 describes the protocol of an acute hemorrhage and volume replacement with lactated ringers or TgHb in some embodiments.

FIG. 17 indicates results mean arterial pressure and heart rate absolute values and relative to baseline following an acute hemorrhage and volume replacement of lactated ringers or TgHb in some embodiments.

FIG. 18 indicates results of microcirculatory arterioles' diameter and flow relative to baseline following an acute hemorrhage and volume replacement of lactated ringers or TgHb in some embodiments. The micro vessels were separated by diameter ≤50 μm or >50 μm. † is significant to baseline.

FIG. 19 indicates results of microcirculatory venules' diameter and flow relative to baseline following an acute hemorrhage and volume replacement of lactated ringers or TgHb in some embodiments. The micro vessels were separated by diameter 20<x<80 μm. † is significant to baseline.

FIG. 20 indicates results of functional capillary density relative to baseline following an acute hemorrhage and volume replacement of lactated ringers or TgHb in some embodiments, † is significant to baseline.

FIG. 21 indicates levels of oxygen saturation in the red blood cells in the arterioles and venules over 60 minutes of recovery after resuscitation with lactated ringers or TgHb in some embodiments.

FIG. 22 illustrates the temporal progression of oxygen saturation in a hamster following an acute hemorrhage and volume replacement of lactated ringers or TgHb in some embodiments.

FIG. 23 indicates levels of cardiac CRP, cardiac IL-6, cardiac IL-1, cardiac TNF-α, cardiac MCP-1 and cardiac Troponin proteins that can indicate one or more status of the cardiac function and cardiac inflammation in hamster following an acute hemorrhage and volume replacement of lactated ringers or TgHb in some embodiments.

FIG. 24 indicates levels of plasma BUN, urine NGAL and urine creatinine, which can be based on to speculate one or more kidney functions following an acute hemorrhage and volume replacement of lactated ringers or TgHb in some embodiments.

FIG. 25 indicates concentrations of renal IL-6, renal IL-1, CXCL1 and IL-10, which can be based on to speculate one or more kidney inflammation in hamsters following an acute hemorrhage and volume replacement of lactated ringers or TgHb in some embodiments.

FIG. 26 indicates Hepatic CXCL1, plasma AST and plasma ALT concentrations, which can be based on to speculate one or more liver functions in hamsters following an acute hemorrhage and volume replacement of lactated ringers or TgHb in some embodiments.

FIG. 27 indicates concentrations of plasma IL-6, plasma CXCL1, and plasma IL-10 in some embodiments, which can be evaluated to speculate endothelium health in hamsters following an acute hemorrhage and volume replacement of lactated ringers or TgHb.

FIG. 28 indicates concentrations of ferritin and bilirubin in hamsters following an acute hemorrhage and volume replacement of lactated ringers or TgHb in some embodiments.

FIG. 29 indicates concentrations of plasma epinephrine and plasma norepinephrine which can be evaluated to speculate nervous system health in hamsters in some embodiments.

FIG. 30 describes the hypovolemic infusion protocol in Golden Syrian hamsters in some embodiments.

FIG. 31 indicates results mean arterial pressure and heart rate relative to a baseline following a hypovolemic infusion of R-State or T-State TgHb in some embodiments.

FIG. 32 indicate results of shear rate versus shear stress of R-State and T-State TgHb over a variety of shear rates starting from 0 s⁻¹ and up to 1000 s⁻¹ using a plate and cone geometry in some embodiments.

FIG. 33 indicates results of log (shear rate) versus log (viscosity) of R-State and T-State TgHb over a variety of shear rates starting from 0 s⁻¹ and up to 1000 s⁻¹ using a plate and cone geometry.

FIG. 34 indicates results of Pharmacokinetics and oxidation rate of R-State and T-State TgHb from 0 hours up to 48 hours taking measurements of plasma Hb and plasma metHb at 0, 2, 4, 8, 12, 24, and 48 hours.

FIG. 35 indicates results of microcirculatory arterioles and venules' diameter and flow relative to baseline following a hypovolemic infusion of R-State or T-State TgHb in some embodiments. The arterioles were separated by diameter ≤50 μm or >50 μm. The venules were separated by diameter 20 μm<x<100. † is significant to baseline.

FIG. 36 indicates results of functional capillary density relative to baseline following the hypovolemic infusion of R-State or T-State TgHb. † is significant to baseline.

FIG. 37 indicates levels of cardiac CRP, cardiac IL-6, cardiac IL-1, cardiac TNF-α, cardiac MCP-1 and cardiac Troponin proteins that can indicate one or more status of the cardiac function and cardiac inflammation in hamster in some embodiments

FIG. 38 indicates levels of plasma BUN, urine NGAL and urine creatinine, which can be based on to speculate one or more kidney functions in some embodiments.

FIG. 39 indicates concentrations of renal IL-6, renal IL-1, CXCL1 and IL-10, which can be based on to speculate one or more kidney inflammation in hamsters in some embodiments.

FIG. 40 indicates Hepatic CXCL1, plasma AST and plasma ALT concentrations, which can be based on to speculate one or more liver functions in hamsters in some embodiments.

FIG. 41 indicates concentrations of plasma IL-6, plasma CXCL1, and plasma IL-10, which can be evaluated to speculate endothelium health in hamsters in some embodiments.

FIG. 42 indicates concentrations of ferritin and bilirubin in hamsters in some embodiments.

FIG. 43 indicates concentrations of plasma epinephrine and plasma norepinephrine which can be evaluated to speculate nervous system health in hamsters in some embodiments.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

### Overview of Disclosure

The present disclosure relates to methods for blood engineering, and particularly for artificial blood.

### Definitions and Interpretation

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

Other examples of implementations will become apparent to the person skilled in the art in view of the teachings of the present description and as such, will not be further described here.

Note that titles or subtitles may be used throughout the present disclosure for the convenience of the reader, but in no way should these limit the scope of the invention. Moreover, certain theories may be proposed and disclosed herein; however, in no way should they, whether they are right or wrong, limit the scope of the invention so long as the invention is practiced according to the present disclosure without regard for any particular theory or scheme of action.

Any and all references cited throughout the specification are hereby incorporated by reference in their entirety for all purposes.

It will be understood by those of skill in the art that throughout the present specification, the term “a” used before a term encompasses embodiments containing one or more of what the term refers to. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions, will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

As used herein, the term “artificial blood”, which is also referred to herein as “synthetic blood” and “blood substitute”, refers a fluid that is not natural blood and that is to replicate or mimic a variety of functions of natural blood, such the oxygen-carrying function of natural blood.

### Detailed Description of Aspects and Embodiments of the Disclosure

With the shortage of blood looming over the growing demand for transfusions, the need to develop blood substitutes is exponentially increasing. A plethora of ideas has been explored over the last 60 years.

Artificial blood, also known as synthetic blood or blood substitute, is a medical technology designed to attempt to replicate or mimic a variety of functions of natural blood, such the oxygen-carrying function of natural blood. Artificial blood can serve as an alternative to human blood transfusions, addressing critical needs in emergency situations, surgeries, and medical treatments where traditional blood supply may be limited or unavailable. Artificial blood can offer the potential to provide a safe, universal, and readily accessible resource for transfusion therapy, potentially saving more lives and overcoming the challenges associated with blood donation and compatibility issues. Artificial blood is a new technology that is being developed to replace donated blood. It could be used to replace donated blood, which is in short supply. For example, artificial blood can be used to treat anemia, a condition in which the body does not have enough red blood cells. For example, artificial blood can be used to improve the outcomes of surgery and trauma care. For example, artificial blood can be used to treat patients who are allergic to blood transfusions.

In some embodiments, artificial blood can be from a variety of materials, including hemoglobin, perfluorocarbons, and polymers. Artificial blood is still in development. Artificial blood has the potential to replace donated blood in a variety of medical applications.

In some embodiments, artificial blood can be based on hemoglobin-based oxygen carriers (HBOCs). HBOCs are made from hemoglobin, which is the protein that carries oxygen in human blood. In some embodiments, artificial blood can be based on perfluorocarbons (PFCs). PFCs are synthetic fluids that can dissolve oxygen and carbon dioxide.

In some embodiments, HBOCs have been shown to be effective in transporting oxygen. In some cases, they can also be toxic to the body. PFCs can be less toxic than HBOCs, but they may not be as effective at transporting oxygen in some cases.

Oxygen carrying blood substitutes based on Hb have been studied for 60 years, still with no formulation available for clinical use. In fact, despite encouraging animal studies, clinical trials have not been considered sufficiently effective, revealing complex biological effects. These problems have made Hb-based red cell substitutes an elusive goal.

Hemoglobin (Hb) is the main component of red blood cells that transports oxygen from the lungs to all parts of the body. It is made up of two sets of two protein molecules, called dimers, and each dimer weighs approximately 32 kDa. Each dimer consists of an Alpha and Beta subunit, with each subunit containing a protein chain and a heme molecule. Hemoglobin has the potential to be a useful replacement for blood transfusions as it can deliver oxygen to tissues.

Acellular hemoglobin (Hb) may not be suitable to be injected to transport oxygen in vivo. In some embodiments, The Hb can be modified to confer biophysical and physiological properties to reduce or prevent Hb toxicity.

Even though earlier attempts to develop HBOCs (Hemoglobin based oxygen carriers) seemed plausible, a few came to fruition due to the unsatisfactory performance and lack of scientific knowledge about oxygen transport and extraction from blood. Common issues associated with HBOCs in the past revolved around the molecular stability of the hemoglobin (Hb). (Alayash, A. I. Hemoglobin-Based Blood Substitutes and the Hazards of Blood Radicals. Free Radic. Res. 2000, 33 (4), 341-348; Buehler, P. W.; Alayash, A. I. All Hemoglobin-Based Oxygen Carriers Are Not Created Equally. Biochim. Biophys. Acta BBA-Proteins Proteomics 2008, 1784 (10), 1378-1381. https://doi.org/10.1016/j.bbapap.2007.12.009; and Alayash, A. I. Hemoglobin-Based Blood Substitutes: Oxygen Carriers, Pressor Agents, or Oxidants? Nat. Biotechnol. 1999, 17 (6), 545-549.) Although a variety of molecular modifications lead to more stable Hb polymers that effectively minimized renal clearance of the protein, they still were not feasible due to economic reasons as many were done using recombinant technology. (Buehler et al. Biochim. Biophys. Acta BBA-Proteins Proteomics 2008; Varnado, C. L.; Mollan, T. L.; Birukou, I; Smith, B. J. Z.; Henderson, D. P.; Olson, J. S. Development of Recombinant Hemoglobin-Based Oxygen Carriers. Antioxid. Redox Signal. 2013, 18 (17), 2314-2328. https://doi.org/10.1089/ars.2012.4917.)

However, protein engineering by post-translation modification led to the creation of high-molecular-weight (MW) Hb molecule through polymerization. A notable hemoglobin-based oxygen carrier is Hemopure (HBOC-201). Hemoglobin-based oxygen carrier-201 (HBOC-201) [hemoglobin glutamer-250 (bovine); Hemopure, HbO2 Therapeutics LLC, Souderton PA 18964, USA] is a solution of purified, glutaraldehyde-polymerized, stroma-free, bovine Hb. (Hughes, G. S. J.; Francome, S. F.; Antal, E. J.; Adams, W. J.; Locker, P. K.; Yancey, E. P.; Jacobs, E. E. J. Hematologic Effects of a Novel Hemoglobin-Based Oxygen Carrier in Normal Male and Female Subjects. J. Lab. Clin. Med. 1995, 126 (5), 444-451.) Through yielding high MW Hb fractions, an increase in viscosity and colloidal osmotic pressure (COP) were observed with increasing cross-linking densities. The large polymerized hemoglobin molecule had a direct impact on the proximity to the vascular endothelium and its molecular diffusivity, thus decreasing nitric oxide (NO) scavenging (preventing induction of vasoconstriction). (Meng, F.; Kassa, T.; Jana, S.; Wood, F.; Zhang, X.; Jia, Y.; D'Agnillo, F.; Alayash, A. I. Comprehensive Biochemical and Biophysical Characterization of Hemoglobin-Based Oxygen Carrier Therapeutics: All HBOCs Are Not Created Equally. Bioconjug. Chem. 2018, 29 (5), 1560-1575. https://doi.org/10.1021/acs.bioconjchem.8b00093; Eich, R. F.; Li, T.; Lemon, D. D.; Doherty, D. H.; Curry, S. R.; Aitken, J. F.; Mathews, A. J.; Johnson, K. A.; Smith, R. D.; Phillips, George N.; Olson, J. S. Mechanism of NO-Induced Oxidation of Myoglobin and Hemoglobin. Biochemistry 1996, 35 (22), 6976-6983. https://doi.org/10.1021/bi960442g; Olson, J. S.; Eich, R. F.; Smith, L. P.; Warren, J. J.; Knowles, B. C. Protein Engineering Strategies for Designing More Stable Hemoglobin-Based Blood Substitutes. Artif. Cells Blood Substit. Biotechnol. 1997, 25 (1-2), 227-241.)

Nonetheless, the use of this HBOC as well as others has been shown in controversy after the meta-analysis of cell-free hemoglobin-based blood substitutes and risk of myocardial infarction (MI) and death was released. (Natanson, C.; Kern, S. J.; Lurie, P.; Banks, S. M.; Wolfe, S. M. Cell-Free Hemoglobin-Based Blood Substitutes and Risk of Myocardial Infarction and Death: A Meta-Analysis. JAMA 2008, 299 (19), 2304-2312. https://doi.org/10.1001/jama.299.19.jrv80007.) This and several other editorials commenting on the potential risks caused widespread controversy over the safety of clinical patients using HBOC-201 that was legalized in South Africa in 2001. (Sarani, B.; Gracias, V. Hemoglobin-Based Blood Substitutes and Risk of Myocardial Infarction and Death. JAMA 2008, 300 (11), 1297-1298; author reply 1298-1299. https://doi.org/10.1001/jama.300.11.1297-b; Cao, M.; Zhao, Y.; He, H.; Yue, R.; Pan, L.; Hu, H.; Ren, Y.; Qin, Q.; Yi, X.; Yin, T.; Ma, L.; Zhang, D.; Huang, X. New Applications of HBOC-201: A 25-Year Review of the Literature. Front. Med. 2021, 8, 794561. https://doi.org/10.3389/fmed.2021.794561.)

The use of HBOC for clinical use is under reasearch and development. The need for some kind of blood substitute increases from year to year. Thus, research on other potential bioconjugated HBOC solutions has continued.

The current standard to generate bioconjugates of hemoglobin uses chemical ligation of glutaraldehyde, a pH-dependent, and temperature sensitive reaction. (Migneault, I.; Dartiguenave, C.; Bertrand, M. J.; Waldron, K. C. Glutaraldehyde: Behavior in Aqueous Solution, Reaction with Proteins, and Application to Enzyme Crosslinking. BioTechniques 2004, 37 (5), 790-796, 798-802. https://doi.org/10.2144/04375RV01.) In Migneault et al., reactions of glutaraldehyde with proteins involve principally the lysine residues in the relative amounts of 4 mol of glutaraldehyde to one of lysine. The products of the interaction of glutaraldehyde with 6-aminohexanoic acid and with a-N-acetyl-lysine were found to be tetrasubstituted pyridinium salts and it was suggested that similar structures are present in the cross-linked products formed by the reaction of glutaraldehyde with proteins. This style of bioconjugation requires large amounts of glutaraldehyde to hemoglobin ratios, previous results suggest that a 36:1 glutaraldehyde to hemoglobin ratio is necessary to alleviate some of the well-studied toxicities of HBOCs. (Muller, C. R.; Williams, A. T.; Munoz, C. J.; Eaker, A. M.; Breton, A. N., Palmer, A. F.; Cabrales, P. Safety Profile of High Molecular Weight Polymerized Hemoglobins. Transfusion (Paris) 2021, 6/(1), 212-224. https://doi.org/10.1111/trf.16157; Williams, A. T.; Muller, C. R.; Eaker, A. M.; Bolden-Rush, C.; Palmer, A. F.; Cabrales, P. Polymerized Hemoglobin Maintains Cardiac Function After Extreme Anemia. FASEB J. 2020, 34 (S1), 1-1. https://doi.org/10.1096/fasebj.2020.34.s1.06291.) Even though chemical ligation is effective, enzyme-promoted bioconjugation provides a viable alternative to it. Enzymes can be easily isolated from cell culture, are cost-effective, environmentally friendly, and avoid the use of organic solvents and harmful chemicals. (Lobba, M. J.; Fellmann, C.; Marmelstein, A. M.; Maza, J. C.; Kissman, E. N.; Robinson, S. A.; Staahl, B. T.; Urnes, C.; Lew, R. J.; Mogilevsky, C. S.; Doudna, J. A.; Francis, M. B. Site-Specific Bioconjugation through Enzyme-Catalyzed Tyrosine-Cysteine Bond Formation. ACS Cent. Sci. 2020, 6 (9), 1564-1571. https://doi.org/10.1021/acscentsci.0c00940; Kalia, J.; Raines, R. T. Advances in Bioconjugation. Curr. Org. Chem. 2010, 14 (2), 138-147. https://doi.org/10.2174/138527210790069839.)

In some embodiments, polymerization of the Hb can extend the retention and circulation time enabling the polymerized Hb (polyHb) to temporary substitute for oxygen carrying properties of red blood cells.

The present disclosure is related to an approach to cross-link Hb to generate biocompatible and homogeneous Hb polymers capable of transport and delivery oxygen to tissues.

The present disclosure is related to an artificial blood that can be relatively more heat stable and can have a longer shelf life compared to a natural blood. In some embodiments, the artificial blood can be used for preserving and transporting live tissue or organ, for example, for transplantation.

In some embodiments, an approach to cross-link the Hb can be the use of an enzyme that can catalyze the formation of an isopeptide bond between the two Hbs, such as γ-carboxamide groups (—(C═O)NH₂) of glutamine residue side chains and the ε-amino groups (⁻NH₂) of lysine residue side chains with subsequent release of ammonia (NH₃). In the case of polymerization of the Hb, various enzymes can be used.

The percentage of Hb cross-linking can be determined by any suitable method including using high-pressure liquid chromatography (HPLC) to determine the percent of total cross-linked Hb, percent of the cross-linked tetramer, percent of the non-cross-linked tetramer, and percent of cross-linked higher-order species (e.g., cross-linked species larger than that of a tetramer). A Waters Protein-Pak (Waters Corporation, Milford, MA) 7.5 mm×300 mm size exclusion column will be used with a mobile phase of 50 mM Tris buffer at pH 7.2 with 0.5 M MgCl₂ (99% purity). The 0.5 M MgCl₂ mobile phase served to dissociate any non-cross-linked tetrameric Hb into pairs of α-β dimers, while cross-linked Hb remained intact. Hence, non-cross-linked Hb and intramolecularly cross-linked Hb tetramers elute separately. All HPLC chromatograms will be reproducible within a 5% experimental margin of error. All measurements will be conducted at 25° C.

For example, protein-glutamine gama-glutamyltransferase, transglutaminase, factor XIIIa, fibrinoligase, fibrinstabilizing factor, glutaminylpeptide gama-glutamyltransferase, polyamine transglutaminase, tissue transglutaminase, R-glutaminyl-peptide: amine gama-glutamyl transferase.

In some embodiments, an approach to cross-link the ultrapure Hb is the use of transglutaminase enzyme that in nature primarily catalyze the formation of an isopeptide bond between γ-carboxamide groups (—(C═O)NH₂) of glutamine residue side chains and the ε-amino groups (⁻NH₂) of lysine residue side chains with subsequent release of ammonia (NH₃).

In some embodiments, transglutaminases comprise a class of protein-glutamine γ-glutamyltransferases (EC 2.3.2.13), bond-forming enzymes that catalyze the attachment of primary amines to protein- or peptide-bound-carboxamides under ammonia release. This transamination involves the glutamine side chains as acyl donors, and the ε-amino groups of lysines as acyl acceptors resulting in stable inter or intramolecular γ-glutamyl-8-lysine isopeptide linkage. (Griffin, M.; Casadio, R., Bergamini, C. M. Transglutaminases: Nature's Biological Glues. Biochem. J. 2002, 368 (Pt 2), 377-396. https://doi.org/10.1042/BJ20021234.) In eukaryotes, transglutaminases are found in many phylogenetic taxonomic groups, including plants, animals, and fungi, where they participate in various physiological and cellular processes, among them wound healing, male sperm maturation, apoptotic death pathways, macrophage endocytosis or cell-matrix assembly. (Duarte, L.; Matte, C. R.; Bizarro, C. V., Ayub, M. A. Z. Transglutaminases: Part I-Origins, Sources, and Biotechnological Characteristics. World J. Microbiol. Biotechnol. 2020, 36 (1), 15. https://doi.org/10.1007/s11274-019-2791-x.) Furthermore, prokaryotic transglutaminases have been found in microorganisms where they are involved in sporulation and the formation of aerial hyphae. (Griffin et al., Biochem. J. 2002; Duarte et al. World J. Microbiol. Biotechnol. 2020). Compared to their eukaryotic counterparts, bacterial transglutaminases possess many advantages like small size, cofactor independency, improved performance, high stability, and reduced deamination activity. Therefore, it has become a field of interest for biochemical application for site-specific bioconjugation.

In some embodiments, Hb alone or combined with other proteins can be effectively modified with transglutaminase (TG); and TG can be inactivated at temperatures inferior to Hb denaturing. Thus, the possibility of creating a library of TG polymeric Hb (TGPolyHb) with different molecular sizes and O2 affinities, either modifying Hb in the tense (deoxy) or relaxed (oxyHb) quaternary state becomes a means of evaluating a potential new hemoglobin-based oxygen carrier. In this study, we begin to explore the use of transglutaminase polymerized hemoglobin (TgHb) at physiological saturation conditions (86%) and comparing it against the microcirculatory responses, organ functionality, and organ inflammation to HBOC-201 and stroma free hemoglobin.

In some embodiments, transglutaminase may be used as the cross-linker as it produces rapid intermolecular and limited intramolecular cross-linking, resulting in stabilized tetramer and consistent molecular weight species. In some embodiments, since a transglutaminase or a transglutaminase-based enzyme can be derived as or from an enzyme that naturally exists in a living organism, the toxicity of the transglutaminase can be relatively substantially lower than other agents such as glutaraldehyde.

Accordingly, the present disclosure is related to a method comprising introducing a transglutaminase to a solution that contains hemoglobins (e.g., oxygenated, deoxygenated, oxidized hemoglobin, human, and/or bovine, etc) to polymerize at least some of the hemoglobins to form polymerized hemoglobins. The present disclosure is related to an aqueous solution comprising the composition comprising hemoglobins including polymerized hemoglobins; and at least a trace amount of transglutaminase.

In some embodiments, the transglutaminase may be a calcium-independent transglutaminase.

In some embodiments, microbial transglutaminase that are calcium-independent can be used. Microbial transglutaminases that are calcium-independent are a type of enzyme that catalyze a chemical reaction known as an acyl transfer. This reaction involves the transfer of acyl groups from glutaminyl residues found in proteins and peptides to various primary amines, such as lysyl residues, in order to form monosubstituted y-amide bonds. As a result of this reaction, transglutaminases can create glutamyl-lysine linkages between different proteins. For this cross-linking to occur, specific glutaminyl residues are to be present and susceptible to the action of transglutaminases, while the accepting lysyl residues are to be in a favorable stereochemical position. Overall, this cross-linking reaction is highly specific.

Calcium-dependent transglutaminases are widely found in mammals and are thought to catalyze many essential biological reactions. For example, they can facilitate the cross-linking of vertebrate fibrin by plasma transglutaminase (also known as blood coagulation Factor XIIa), the clotting of seminal plasma by transglutaminase from the anterior prostate gland, the cross-linking of hair proteins by hair follicle transglutaminase, and the cross-linking of keratin by epidermal transglutaminase.

Transglutaminases (TG) can carry out various biological functions that are generally attributed to their protein-modifying activity, in some instances their biological function is due to specialized non-catalytic actions, such as scaffolding of the cytoskeleton to maintain membrane integrity (for example, erythrocyte band 4.2, which is a catalytically inactive ninth member of the mammalian TG family), cell adhesion and possibly signal transduction (for example, TG2).

In some embodiments, the utilization of TG to polymerize hemoglobin to Transglutaminase-polymerized hemoglobin (TgHb or PolyTghHb) as another class of HBOCs.

In some embodiments, various TG can be used to polymerize hemoglobin.

In some embodiments, hemoglobin can be obtained from different sources. For example, hemoglobin can be derived from a human blood. For example, hemoglobin can be derived from expired blood from a blood bank. For example, hemoglobin can be derived from another species, such as an animal, a mammalian or any types of life form such as eukaryotic and prokaryotic cells. For example, hemoglobin can be produced or synthesized using various chemical and biological processes. There can be many different methods of protein manufacturing to manufacture hemoglobins. For example, the methods can include bacterial expressions, yeast expressions, mammalian cell expressions, insect cell expressions, plant cell expressions, animal cell expressions, and cell-free expressions that do not involve live cells. In addition to the methods listed above, there are a number of other methods that can be used to produce proteins. For example, the methods may include chemical synthesis and recombinant DNA technology. A protein production process can depend on a number of factors, including the desired purity of the protein, the cost of production, and the time required to produce the protein.

In some embodiments, various methods of polymerizing hemoglobin or controlling or influencing the structure of the hemoglobin polymer can be used. For example, different types of enzymes or any combination thereof can be used. For example, different parameters can be controlled, such as temperature, concentrations, and time.

For example, different hemoglobin protein dispersions can be prepared such as at either 10% or 20% solids content (w/v). For example, pH of the dispersions can be controlled or adjusted such as pH 7.5. For example, condition of mixing protein substrate with transglutaminase can be controlled, such as agitating speed and incubation temperature that can influence the rate of enzyme activities. For example, the ratio between protein substrate such as hemoglobin and an enzyme, such as transglutaminase, can be controlled, such as at a ratio of approximately 5 units per gram of hemoglobin.

In some embodiments, enzyme concentration, hemoglobin concentration, temperature, and time of reaction can be controlled and adjusted by various factors or considerations. For example, one or more of these parameters can be evaluated for a central composite design with factors to find the optimum conditions. For example, experiments can be performed, with different concentrations of transglutaminase, temperature, and Hb concentrations.

The present disclosure is related to a method comprising introducing a transglutaminase to a solution that contains a plurality of hemoglobins to polymerize at least a portion of the plurality of the hemoglobins to form polymerized hemoglobins.

In some embodiments, prior to introducing the transglutaminase, solid content of the plurality of hemoglobins in the solution can be from about 1 g/dL to about 25 g/dL.

In some embodiments, prior to introducing the transglutaminase, solid content of the plurality of hemoglobins in the solution can be from about 5 g/dL to about 20 g/dL.

In some embodiments, prior to introducing the transglutaminase, solid content of the plurality of hemoglobins in the solution can be from about 10 g/dL to about 15 g/dL.

In embodiments, the solid content of the plurality of hemoglobins in the solution may be up to 10 g/dL, up to 12 g/dL, up to 14 g/dL, up to 16 g/dl, up to 20 g/dL; not less than 20 g/dL, not less than 15 g/dL, not less than 12 g/dL, not less than 10 g/dL, not less than 8 g/dL, not less than 5 g/dL, not less than 2 g/dL, not less than 1 g/dL; or from 1 g/dL to 10 g/dL, 1 g/dL to 5 g/dL, 2 g/dL to 12 g/dL, 6 g/dL to 18 g/dL, 5 g/dL to 15 g/dL, 5 g/dL to 25 g/dL, 10 g/dL to 20 g/dL, 1 g/dL to 20 g/dL, 1 g/dL to 15 g/dL, 5 g/dL to 10 g/dL, or 10 g/dL to 25 g/dL.

In some embodiments, introducing the transglutaminase may comprise introducing the transglutaminase at a ratio of from about 2 to about 10 enzyme units of the transglutaminase per gram of the plurality of hemoglobins.

In some embodiments, introducing the transglutaminase may comprise introducing the transglutaminase at a ratio of from about 5 enzyme units of the transglutaminase per a gram of the plurality of hemoglobins.

In some embodiments, the transglutaminase comprises a calcium-independent transglutaminase.

The present disclosure is related to a composition comprising a plurality of hemoglobins, wherein at least a portion of the plurality of hemoglobins may comprise polymerized hemoglobins and an amount of transglutaminase.

In some embodiments, the amount of transglutaminase may be a trace amount.

In some embodiments, an aqueous solution may comprise the composition comprising a plurality of hemoglobins, wherein at least a portion of the plurality of hemoglobins may comprise polymerized hemoglobins and an amount of transglutaminase.

In some embodiments, solid content of the plurality of hemoglobins in the solution may be from about 1 g/dL to about 25 g/dL.

In some embodiments, solid content of the plurality of hemoglobins in the solution may be from about 5 g/dL to about 20 g/dL.

In some embodiments, solid content of the plurality of hemoglobins in the solution may be from about 10 g/dL to about 15 g/dL.

In some embodiments, solid content of the plurality of hemoglobins in the solution may be about 20 g/dL.

In embodiments, the solid content of the plurality of hemoglobins in the solution may be up to 10 g/dL, up to 12 g/dL, up to 14 g/dL, up to 16 g/dL, up to 20 g/dL; not less than 20 g/dL, not less than 15 g/dL, not less than 12 g/dL, not less than 10 g/dL, not less than 8 g/dL, not less than 5 g/dL, not less than 2 g/dL, not less than 1 g/dL; or from 1 g/dL to 10 g/dL, 1 g/dL to 5 g/dL, 2 g/dL to 12 g/dL, 6 g/dL to 18 g/dL, 5 g/dL to 15 g/dL, 5 g/dL to 25 g/dL, 10 g/dL to 20 g/dL, 1 g/dL to 20 g/dL, 1 g/dL to 15 g/dL, 5 g/dL to 10 g/dL, or 10 g/dL to 25 g/dL.

In some embodiments, the aqueous solution may exhibit a colloid osmotic pressure of less than about 50 mmHg.

In some embodiments, the aqueous solution may exhibit a colloid osmotic pressure of less than about 40 mmHg.

In some embodiments, the aqueous solution may exhibit a colloid osmotic pressure of from about 30 mmHg to about 35 mmHg.

In some embodiments, wherein solid content of the plurality of hemoglobins in the solution may be about 10 g/dL.

As described herein, hemoglobins can have two main states, the “R State” and “T State. In the R state (relaxed state), hemoglobin has a higher affinity for oxygen, meaning it binds oxygen more readily. In contrast, in the T state (tense state), hemoglobin has a lower affinity for oxygen, making it less likely to bind oxygen molecules. The transition between these two states is crucial for the efficient loading and unloading of oxygen in the lungs and tissues, respectively.

Fully oxygenated hemoglobin (Hb) is in the R state. In the R state, hemoglobin has a higher affinity for oxygen, allowing it to bind oxygen molecules more readily. As oxygen binds to hemoglobin in the lungs, it induces a conformational change from the T state to the R state, facilitating the loading of oxygen onto hemoglobin. In tissues where oxygen is needed, the release of oxygen causes hemoglobin to transition back to the T state, allowing for efficient oxygen unloading. The T state refers to the conformation of hemoglobin when it is fully deoxygenated, meaning it has released all its bound oxygen molecules. In this state, hemoglobin has a lower affinity for oxygen, making it more likely to release oxygen molecules to the surrounding tissues.

Whether hemoglobins are in an “oxygenated” state (e.g., fully oxygenated) or a deoxygenated state (e.g., fully deoxygenated state) can be determined using methods known in the art, for example, using a Hemox-Analyzer (TCSScientific Corporation, New Hope, PA) at 37° C.

In some embodiments, when an oxygenation state of the polymerized hemoglobins is about a fully oxygenated state of the polymerized hemoglobins, a viscosity of the aqueous solution at a shear rate of about 100 s⁻¹ at about 37° C. can be from about 35 cP to about 50 cP.

In some embodiments, when the oxygenation state is about the fully oxygenated state of the polymerized hemoglobins, the viscosity of the aqueous solution at the shear rate of about 100 s⁻¹ at about 37° C. can be about 42 cP.

In some embodiments, a viscosity of the aqueous solution at a shear rate of about 100 s⁻¹ may change at least by about 25 cP or more when an oxygenation state of the polymerized hemoglobins changes from about a deoxygenated state to about a fully oxygenated state.

In some embodiments, the transglutaminase may comprise a calcium-independent transglutaminase.

The present disclosure is related to an aqueous solution comprising a plurality of hemoglobins, wherein at least a portion of the plurality of hemoglobins can comprise transglutaminase-polymerized hemoglobins (TgHbs), and wherein a viscosity of the aqueous solution at a shear rate of about 100 s⁻¹ at about 37° C. can change by about 25 cP or more when an oxygenation state of the TgHbs changes from about a deoxygenated state to about a fully oxygenated state.

In some embodiments, the viscosity of the aqueous solution at the shear rate of about 100 s⁻¹ at about 37° C. can change by about 30 cP when the oxygenation state of the TgHbs changes from about deoxygenated state to about fully oxygenated state.

In some embodiments, solid content of the plurality of hemoglobins in the solution can be from about 1 gram per g/dL to about 25 g/dL.

In some embodiments, solid content of the plurality of hemoglobins in the solution can be from about 5 gram per g/dL to about 20 g/dL.

In some embodiments, solid content of the plurality of hemoglobins in the solution can be from about 10 gram per g/dL to about 15 g/dL.

In some embodiments, solid content of the plurality of hemoglobins in the solution can be from about 10 gram per deci-Liter (g/dL) to about 20 g/dL.

In some embodiments, solid content of the plurality of hemoglobins in the solution can be about 20 g/dL.

In embodiments, the solid content of the plurality of hemoglobins in the solution may be up to 10 g/dL, up to 12 g/dL, up to 14 g/dL, up to 16 g/dL, up to 20 g/dL; not less than 20 g/dL, not less than 15 g/dL, not less than 12 g/dL, not less than 10 g/dL, not less than 8 g/dL, not less than 5 g/dL, not less than 2 g/dL, not less than 1 g/dL; or from 1 g/dL to 10 g/dL, 1 g/dL to 5 g/dL, 2 g/dL to 12 g/dL, 6 g/dL to 18 g/dL, 5 g/dL to 15 g/dL, 5 g/dL to 25 g/dL, 10 g/dL to 20 g/dL, 1 g/dL to 20 g/dL, 1 g/dL to 15 g/dL, 5 g/dL to 10 g/dL, or 10 g/dL to 25 g/dL.

In some embodiments, the aqueous solution may exhibit a colloid osmotic pressure of less than about 50 mmHg.

In some embodiments, the aqueous solution may exhibit a colloid osmotic pressure of less than about 40 mmHg.

In some embodiments, the aqueous solution may exhibit a colloid osmotic pressure of from about 30 mmHg to about 35 mmHg.

In some embodiments, solid content of the plurality of hemoglobins in the solution can be about 10 g/dL.

In some embodiments, when the oxygenation state is around the fully oxygenated state of the TgHbs, the viscosity of the aqueous solution at a shear rate of about 100 s⁻¹ at about 37° C. can be from about 35 cP to about 50 cP.

In some embodiments, when the oxygenation state is around the fully oxygenated state of the TgHbs, the viscosity at the shear rate of about 100 s⁻¹ at about 37° C. can be about 42 cP.

The present disclosure is related to a method comprising introducing a transglutaminase to a solution that contains hemoglobins, to polymerize at least some of the hemoglobins to form polymerized hemoglobins.

In some embodiments, before introducing the transglutaminase, the solid content of the hemoglobins in the solution may be from about 10 gram per deci-Liter (g/dL) to about 20 g/dL, for example, up to 10 g/dL, up to 12 g/dL, up to 14 g/dL, up to 16 g/dL, up to 20 g/dL; not less than 20 g/dL, not less than 15 g/dL, not less than 12 g/dL, not less than 10 g/dL; or from 6 g/dL to 18 g/dL, 5 g/dL to 15 g/dL, 5 g/dL to 20 g/dL, 10 g/dL to 20 g/dL, or 5 g/dL to 10 g/dL.

In some embodiments, introducing the transglutaminase may comprise introducing the transglutaminase at a ratio of from about 2 to about 10 enzyme units of the transglutaminase per gram of the hemoglobins.

In some embodiments, introducing the transglutaminase may comprise introducing the transglutaminase at a ratio of from about 5 enzyme units of the transglutaminase per gram of the hemoglobins.

In some embodiments, the transglutaminase may be a calcium-independent transglutaminase.

The present disclosure is related to a composition comprising hemoglobins including polymerized hemoglobins; and at least a trace amount of transglutaminase.

The present disclosure is related to an aqueous solution comprising the composition comprising hemoglobins including polymerized hemoglobins; and at least a trace amount of transglutaminase.

In some embodiments, the solid content of the hemoglobins in the solution may be from about 10 gram per deci-Liter (g/dL) to about 20 g/dL, for example, up to 10 g/dL, up to 12 g/dL, up to 14 g/dL, up to 16 g/dL, up to 20 g/dL; not less than 20 g/dL, not less than 15 g/dL, not less than 12 g/dL, not less than 10 g/dL; or from 6 g/dL to 18 g/dL, 5 g/dL to 15 g/dL, 5 g/dL to 20 g/dL, 10 g/dL to 20 g/dL, or 5 g/dL to 10 g/dL.

In some embodiments, the solid content of the hemoglobins in the solution may be about 20 g/dL.

In some embodiments, the aqueous solution may exhibit the colloid osmotic pressure of less than about 50 mmHg.

In some embodiments, the aqueous solution may exhibit the colloid osmotic pressure of less than about 40 mmHg.

In some embodiments, the aqueous solution may exhibit the colloid osmotic pressure of from about 30 mmHg to about 35 mmHg.

In some embodiments, the solid content of the hemoglobins in the solution may be about 10 g/dL.

In some embodiments, the viscosity of the aqueous solution at the shear rate of about 100 s⁻¹ may be from about 35 cP to about 50 cP.

In some embodiments, the viscosity of the aqueous solution at the shear rate of about 100 s⁻¹ may be about 42 cP.

In some embodiments, the viscosity of 42 cP at the shear rate of about 100 s⁻¹ may indicate at least close to about fully oxygenated state of the polymerized hemoglobins.

In some embodiments, the viscosity of the aqueous solution at the shear rate of about 100 s⁻¹ may change at least by about 25 cP or more when the oxygenation state of the polymerized hemoglobin polymer changes from about deoxygenated state to about fully oxygenated state.

In some embodiments, the transglutaminase may be a calcium-independent transglutaminase.

The present disclosure is related to an aqueous solution comprising hemoglobins including transglutaminase polymerized hemoglobins (TgHbs), wherein the viscosity of the aqueous solution at the shear rate of about 100 second⁻¹ (s⁻¹) changes by about 25 cP or more when the oxygenation state of the TgHbs changes from about a deoxygenated state to about a fully oxygenated state.

In some embodiments, the viscosity of the aqueous solution at the shear rate of about 100 s⁻¹ may change by about 30 cP when the oxygenation state of the TgHbs changes from about deoxygenated state to about fully oxygenated state.

In some embodiments, the solid content of the hemoglobins in the solution may be from about 10 gram per deci-Liter (g/dL) to about 20 g/dL, for example, up to 10 g/dL, up to 12 g/dL, up to 14 g/dL, up to 16 g/dL, up to 20 g/dL; not less than 20 g/dL, not less than 15 g/dL, not less than 12 g/dL, not less than 10 g/dL; or from 6 g/dL to 18 g/dL, 5 g/dL to 15 g/dL, 5 g/dL to 20 g/dL, 10 g/dL to 20 g/dL, or 5 g/dL to 10 g/dL.

In some embodiments, the solid content of the hemoglobins in the solution may be about 20 g/dL.

In some embodiments, the aqueous solution may exhibit the colloid osmotic pressure of less than about 50 mmHg.

In some embodiments, the aqueous solution may exhibit the colloid osmotic pressure of less than about 40 mmHg.

In some embodiments, the aqueous solution may exhibit the colloid osmotic pressure of from about 30 mmHg to about 35 mmHg.

In some embodiments, the solid content of the hemoglobins in the solution may be about 10 g/dL.

In some embodiments, the viscosity of the aqueous solution at the shear rate of about 100 s⁻¹ may be from about 35 cP to about 50 cP.

In some embodiments, the viscosity at the shear rate of about 100 s⁻¹ may be about 42 cP.

In some embodiments, the viscosity of 42 cP at the shear rate of about 100 s⁻¹ may indicate at least close to about fully oxygenated state of the TgHbs.

The disclosure also relates to the following numbered Embodiments in no particular order of importance:

### Embodiment 1

A method comprising:
introducing a transglutaminase to a solution that contains a plurality of hemoglobins to polymerize at least a portion of the plurality of the hemoglobins to form polymerized hemoglobins.

### Embodiment 2

The method of claim 1, wherein, prior to introducing the transglutaminase, solid content of the plurality of hemoglobins in the solution is from about 1 g/dL to about 25 g/dL.

### Embodiment 3

The method of Embodiment 1, wherein, prior to introducing the transglutaminase, solid content of the plurality of hemoglobins in the solution is from about 5 g/dL to about 20 g/dL.

### Embodiment 4

The method of Embodiment 1, wherein, prior to introducing the transglutaminase, solid content of the plurality of hemoglobins in the solution is from about 10 g/dL to about 15 g/dL.

### Embodiment 5

The method of Embodiment 2, wherein introducing the transglutaminase comprises introducing the transglutaminase at a ratio of from about 2 to about 10 enzyme units of the transglutaminase per gram of the plurality of hemoglobins.

### Embodiment 6

The method of Embodiment 5, wherein introducing the transglutaminase comprises introducing the transglutaminase at a ratio of from about 5 enzyme units of the transglutaminase per a gram of the plurality of hemoglobins.

### Embodiment 7

The method of Embodiment 1, wherein the transglutaminase comprises a calcium-independent transglutaminase.

### Embodiment 8

A composition comprising:
a plurality of hemoglobins, wherein at least a portion of the plurality of hemoglobins comprises polymerized hemoglobins; and an amount of transglutaminase.

### Embodiment 9

The composition of Embodiment 8, wherein the amount of transglutaminase is a trace amount.

### Embodiment 10

An aqueous solution comprising the composition of Embodiment 8.

### Embodiment 11

The aqueous solution of Embodiment 10, wherein solid content of the plurality of hemoglobins in the solution is from about 1 g/dL to about 25 g/dL.

### Embodiment 12

The aqueous solution of Embodiment 10, wherein solid content of the plurality of hemoglobins in the solution is from about 5 g/dL to about 20 g/dL.

### Embodiment 13

The aqueous solution of Embodiment 10, wherein solid content of the plurality of hemoglobins in the solution is from about 10 g/dL to about 15 g/dL.

### Embodiment 14

The aqueous solution of Embodiment 11, wherein solid content of the plurality of hemoglobins in the solution is about 20 g/dL.

### Embodiment 15

The aqueous solution of Embodiment 14, wherein the aqueous solution exhibits a colloid osmotic pressure of less than about 50 mmHg.

### Embodiment 16

The aqueous solution of Embodiment 14, wherein the aqueous solution exhibits a colloid osmotic pressure of less than about 40 mmHg.

### Embodiment 17

The aqueous solution of Embodiment 14, wherein the aqueous solution exhibits a colloid osmotic pressure of from about 30 mmHg to about 35 mmHg.

### Embodiment 18

The aqueous solution of Embodiment 11, wherein solid content of the plurality of hemoglobins in the solution is about 10 g/dL.

### Embodiment 19

The aqueous solution of Embodiment 18, wherein, when an oxygenation state of the polymerized hemoglobins is about a fully oxygenated state of the polymerized hemoglobins, a viscosity of the aqueous solution at a shear rate of about 100 s⁻¹ at about 37° C. is from about 35 cP to about 50 cP.

### Embodiment 20

The aqueous solution of Embodiment 19, wherein, when the oxygenation state is about the fully oxygenated state of the polymerized hemoglobins, the viscosity of the aqueous solution at the shear rate of about 100 s⁻¹ at about 37° C. is about 42 cP.

### Embodiment 21

The aqueous solution of Embodiment 18, wherein a viscosity of the aqueous solution at a shear rate of about 100 s⁻¹ changes at least by about 25 cP or more when an oxygenation state of the polymerized hemoglobins changes from about a deoxygenated state to about a fully oxygenated state.

### Embodiment 22

The composition of Embodiment 8, wherein the transglutaminase comprises a calcium-independent transglutaminase.

### Embodiment 23

An aqueous solution comprising:
a plurality of hemoglobins, wherein at least a portion of the plurality of hemoglobins comprises transglutaminase-polymerized hemoglobins (TgHbs), and
wherein a viscosity of the aqueous solution at a shear rate of about 100 s⁻¹ at about 37° C. changes by about 25 cP or more when an oxygenation state of the TgHbs changes from about a deoxygenated state to about a fully oxygenated state.

### Embodiment 24

The aqueous solution of Embodiment 23, wherein the viscosity of the aqueous solution at the shear rate of about 100 s⁻¹ at about 37° C. changes by about 30 cP when the oxygenation state of the TgHbs changes from about deoxygenated state to about fully oxygenated state.

### Embodiment 25

The aqueous solution of Embodiment 23, wherein solid content of the plurality of hemoglobins in the solution is from about 1 gram per g/dL to about 25 g/dL.

### Embodiment 26

The aqueous solution of Embodiment 23, wherein solid content of the plurality of hemoglobins in the solution is from about 5 gram per g/dL to about 20 g/dL.

27. The aqueous solution of Embodiment 23, wherein solid content of the plurality of hemoglobins in the solution is from about 10 gram per g/dL to about 15 g/dL.

### Embodiment 28

The aqueous solution of Embodiment 23, wherein solid content of the plurality of hemoglobins in the solution is from about 10 gram per deci-Liter (g/dL) to about 20 g/dL.

### Embodiment 29

The aqueous solution of Embodiment 28, wherein solid content of the plurality of hemoglobins in the solution is about 20 g/dL.

### Embodiment 30

The aqueous solution of Embodiment 29, wherein the aqueous solution exhibits a colloid osmotic pressure of less than about 50 mmHg.

### Embodiment 31

The aqueous solution of Embodiment 29, wherein the aqueous solution exhibits a colloid osmotic pressure of less than about 40 mmHg.

### Embodiment 32

The aqueous solution of Embodiment 29, wherein the aqueous solution exhibits a colloid osmotic pressure of from about 30 mmHg to about 35 mmHg.

### Embodiment 33

The aqueous solution of Embodiment 28, wherein solid content of the plurality of hemoglobins in the solution is about 10 g/dL.

### Embodiment 34

The aqueous solution of Embodiment 33, wherein, when the oxygenation state is around the fully oxygenated state of the TgHbs, the viscosity of the aqueous solution at a shear rate of about 100 s⁻¹ at about 37° C. is from about 35 cP to about 50 cP.

### Embodiment 35

The aqueous solution of Embodiment 34, wherein, when the oxygenation state is around the fully oxygenated state of the TgHbs, the viscosity at the shear rate of about 100 s⁻¹ at about 37° C. is about 42 cP.

### Embodiment 36

A method comprising: polymerizing a plurality of hemoglobin molecules in the presence of transglutaminase to form polymerized hemoglobin molecules.

### Embodiment 37

Polymerized hemoglobins comprising one or more γ-glutamyl-¿-lysine isopeptide linkages.

### Embodiment 38

A plurality of hemoglobins, wherein at least a portion of the plurality of hemoglobins comprises transglutaminase-polymerized hemoglobins (TgHbs) comprising one or more γ-glutamyl-8-lysine isopeptide linkages.

### Embodiment 39

A plurality of hemoglobins, wherein at least a portion of the plurality of hemoglobins comprises transglutaminase-polymerized hemoglobins (TgHbs) comprising one or more γ-glutamyl-¿-lysine isopeptide linkages made by the method of Embodiment 1.

### EXAMPLES

The following methods were employed in the Examples described herein.

### Example 1: Preparation of TgHb

### Materials

Calcium-independent microbial transglutaminase was purchased from Ajinomoto (Japan). The enzyme presented mean enzymatic activity of 200 U/g, as per manufacturer's data. The enzyme was used in the original form without further purification.

The Blood Bank provided outdated packs of human red blood cells (PRBCs) that had been tested for the presence of human immunodeficiency virus (HIV) and hepatitis B antigen. These PRBCs were of types A, AB, or O, and were pooled together.

### Hemoglobin Preparation

The PRBCs were then washed four times with chilled normal saline at 4° C. and centrifuged at 5,000 rpm for 15 minutes for each wash. In each wash, the volume of saline used was four times greater than the volume of the PRBCs. To confirm that most of the plasma was removed during the washing process, the albumin concentration was measured using a Human Albumin ELISA Kit (ABCAM: ab179887).

After washing, the red cells were suspended three times the volume of deionized water at 4° C. for 30 minutes to release the hemoglobin (Hb). The solution was then washed with ice-cold deionized water (Epure, Barnstead, Dubuque, IA) to break open the red cells. Toluene was added at 4° C. and mixed vigorously to separate the Hb from the red cell debris. The mixture was left to rest overnight at 4° C. The next day, the aqueous phase containing the Hb was removed and filtered through a 0.22 μm filter. The concentration of Hb and MetHb level were established using Drabkin's method, where all forms of Hb were oxidized into the stable CnMetHb using a mixture of potassium ferricyanide and potassium cyanide. The absorbance was read at 540 nm, and the concentration of total Hb was determined using the Beer-Lambert law. MetHb was calculated by measuring the change in absorption of MetHb to CnMetHb at 635 nm. The filtrate, Stroma-Free Hemoglobin (SFH), was then concentrated to 10 g/dL by circulating it through three 10-kDa hollow fiber membrane cartridges. The SFH solution was then diafiltered with five volumes of Ringer's acetate, pH 7.4, and transferred to a sterile reservoir.

### MetHb Measurements.

MetHb levels were determined using a cyanometHb method using spectrophotometery (19) measured using a Cary 3 UV-visible spectrometer (Varian Analytical Instruments, Walnut Creek, CA) as mentioned before.

### HPLC Measurements.

High-pressure liquid chromatography (HPLC) was used to determine the percent of total cross-linked Hb, percent of cross-linked tetramer, percent of non-cross-linked tetramer, and percent of cross-linked higher-order species (i.e., cross-linked species larger than that of a tetramer). A Waters Protein-Pak (Waters Corporation, Milford, MA) 7.5 mm×300 mm size exclusion column was used with a mobile phase of 50 mM Tris buffer at pH 7.2 with 0.5 M MgCl₂ (99% purity). The 0.5 M MgCl₂ mobile phase served to dissociate any non-cross-linked tetrameric Hb into pairs of α-β dimers, while cross-linked Hb remained intact. Hence, non-cross-linked Hb and intramolecularly cross-linked Hb tetramers elute separately. All HPLC chromatograms were reproducible within 5% experimental error. All measurements were conducted at 25° C. In preparation for AFFF-MASLS-DIR experiments, all glassware was carefully cleaned and all solutions were thoroughly filtered through 0.1 μm filters. A typical separation lasted approximately 60 min. A nondissociating mobile phase of 50 mM Tris pH 7.2 was used for all measurements, since dissociating conditions (high salt concentrations) would corrode the internal plumbing of the AFFF chromatography system. This resulted in both cross-linked and non-cross-linked tetramers giving rise to the same molecular weight of 64,000 g/mol.

### Enzymatic Polymerization of Hemoglobin Protein

Hemoglobin protein dispersions were prepared at either 10% or 20% solids content (w/v) and adjusted to pH 7.5. The protein substrate was mixed with microbial transglutaminase at a ratio of approximately 5 units per gram of hemoglobin and slowly agitated in a rotating incubator at 37° C. At timed intervals, samples were taken for SDS-PAGE and OPA analyses. In most cases, the soluble supernatants obtained after centrifugation at 11950 g were tested. Residual protein content in transglutaminase-treated samples that were kept at 37° C. for 24 hours was measured using the BCA method. Control solutions without the enzyme were also evaluated.

### Factorial Design

Enzyme concentration, temperature and time of reaction were evaluated by a central composite design with three factors to find the optimum conditions. A total of 12 experiments were performed, 3 transglutaminase concentration (2, 5, 10), 2 temperature (25 C and 37 C), and 2 Hb concentrations (10 and 20 g/dL). The average molecular weight of the protein solutions subjected to enzyme reaction with transglutaminase were the selected responses.

### SDS-PAGE Electrophoresis for Hemoglobin Protein

To ensure equal loading amounts into each lane, all samples were initially evaluated for protein content using the BCA method. The fractions were then appropriately diluted and mixed (1:1, v/v) with sample buffer [8% SDS, 0.9 M Tris buffer containing 5.0% β-mercaptoethanol (In Vitrogen Inc., Carlsbad, CA)]. Each sample was heated at 100° C. for 10 minutes, which also stops the enzymatic reaction, before loading onto 10-20% Tris-Tricine gradient polyacrylamide gels (In Vitrogen, Inc.). After electrophoresis, proteins were visualized by directly staining the samples with a colloidal Coomassie Blue staining reagent (In Vitrogen, Inc.).

### Example 2: Comparison of hHb, HBOC-201, and TgHb

For the following EXAMPLES 2-10, the efficacy of this hemoglobin-based oxygen carrier using transglutaminase polymerization (TgHb) was tested en-vivo using the Golden Syrian hamsters.

### Materials and Methods

### Hb Preparation

Hb (P68871, P69905) for this study was prepared via tangential flow filtration as previously described. (Palmer, A. F.; Sun, G.; Harris, D. R. Tangential Flow Filtration of Hemoglobin. Biotechnol. Prog. 2009, 25 (1), 189-199. https://doi.org/10.1002/btpr.119). The concentration of Hb was determined spectrophotometrically.

### Polymerization of Hb Using Transglutaminase

Hemoglobin protein dispersions were prepared at either 20% solids content (w/v) and adjusted to pH 7.5. The protein substrate was mixed with microbial transglutaminase at a ratio of approximately 5 units per gram of hemoglobin and slowly agitated in a rotating incubator at 37° C. At timed intervals, samples were taken for SDS-PAGE and OPA analyses. In most cases, the soluble supernatants obtained after centrifugation at 11950 g were tested. Residual protein content in transglutaminase-treated samples that were kept at 37° C. for 24 hours was measured using the BCA method.

### MetHb Measurements.

MetHb levels were determined using a cyanometHb method using spectrophotometery (19) measured using a Cary 3 UV-visible spectrometer (Varian Analytical Instruments, Walnut Creek, CA) as mentioned before.

### Rheology and Colloidal Osmotic Pressure Measurements

The viscosity and colloidal osmotic pressure were measured for transglutaminase polymerization human Hb (TghHb). The viscosity was measured using a computerized cone-plate rheometer at 37° C. (Discovery HR-2; TA Instruments, New Castle, DE). Colloidal osmotic pressure (COP) was measured using a 30-kDa cutoff filter at 22° C. (colloid osmometer model 4420; Wescor, Logan, UT).

### Window Chamber Preparation in Golden Syrian Hamsters

Investigations were performed in 55-65 g male Golden Syrian Hamsters (Charles River Laboratories, Boston, MA) fitted with a dorsal skinfold window chamber. Animal handling and care followed the NIH Guide for the Care and Use of Laboratory Animals. The experimental protocol was approved by the local animal care committee. The hamster window chamber model is widely used for microvascular studies in the unanesthetized state, and the complete surgical technique is described in detail elsewhere. (Colantuoni, A.; Bertuglia, S.; Intaglietta, M. Quantitation of Rhythmic Diameter Changes in Arterial Microcirculation. Am. J. Physiol. 1984, 246 (4 Pt 2), H508-517. https://doi.org/10.1152/ajpheart. 1984.246.4.H508.) The experimental animal was allowed at least 2 days for recovery before the preparation was assessed under the microscope for any signs of edema, bleeding, or unusual neovascularization. Animals were anesthetized again, and arterial and venous catheters filled with a heparinized saline solution (30 IU/ml) were implanted. Catheters were tunneled under the skin, exteriorized at the dorsal side of the neck, and securely attached to the window frame. The microvasculature was examined 3 to 4 days after the initial surgery and only animals with window chambers whose tissue did not present regions of low perfusion, inflammation, and edema were entered into the study. (Tsai, A. G.; Friesenecker, B.; McCarthy, M.; Sakai, H.; Intaglietta, M. Plasma Viscosity Regulates Capillary Perfusion during Extreme Hemodilution in Hamster Skinfold Model. Am. J. Physiol. 1998, 275 (6), H2170-2180)

### Inclusion Criteria

Animals were considered suitable for experiments if systemic parameters were as follows: heart rate (HR)>340 beats/min, mean arterial blood pressure (MAP)>80 mm Hg, systemic hematocrit (Hct)>45%, and arterial O2 partial pressure (pAO2)>50 mm Hg. Additionally, animals with signs of low perfusion, inflammation, edema, or bleeding in their microvasculature were excluded from the study.

### Experimental Setup

The unanesthetized animal was placed in a restraining tube with a longitudinal slit from which the window chamber protruded, then fixed to the microscopic stage for transillumination with the intravital microscope (BX51WI, Olympus, Japan). Animals were given 20 minutes to adjust to the tube environment and images were obtained using a CCD camera (4815, COHU, San Diego, CA). Measurements were carried out using a 40× (LUMPFL-WIR, numerical aperture 0.8, Olympus) water immersion objective.

### Systemic Parameters

The Mean Arterial Pressure (MAP) and Heart Rate (HR) were monitored continuously (MP150, Biopac System Inc., Santa Barbara, CA). Hematocrit (Hct) was measured from centrifuged arterial blood samples taken in heparinized capillary tubes. Hb content was determined spectrophotometrically (B-Hemoglobin, Hemocue, Stockholm, Sweden). Arterial blood was collected in heparinized glass capillaries (50 μL) and immediately analyzed for total hemoglobin concentration (tHb), partial pressure of oxygen (pO2), partial pressure of carbon dioxide (pCO2), and pH (ABL90; Radiometer America, Brea, CA).

### Microhemodynamics

Arteriolar and venular blood flow velocities were measured online by using the photodiode cross-correlation method (Intaglietta, M.; Silverman, N. R.; Tompkins, W. R. Capillary Flow Velocity Measurements in Vivo and in Situ by Television Methods. Microvasc. Res. 1975, 10 (2), 165-179. https://doi.org/10.1016/0026-2862 (75) 90004-7) (Photo Diode/Velocity Tracker Model 102B, Vista Electronics, San Diego, CA). The measured centerline velocity (V) was corrected according to vessel size to obtain the mean RBC velocity. (Lipowsky, H. H.; Zweifach, B. W. Application of the “Two-Slit” Photometric Technique to the Measurement of Microvascular Volumetric Flow Rates. Microvasc. Res. 1978, 15 (1), 93-101. https://doi.org/10.1016/0026-2862 (78) 90009-2.) A video image-shearing method was used to measure vessel diameter (D). (Intaglietta, M.; Tompkins, W. R. Microvascular Measurements by Video Image Shearing and Splitting. Microvasc. Res. 1973, 5 (3), 309-312. (2) Lipowsky, H. H., Zweifach, B. W. Application of the “Two-Slit” Photometric Technique to the Measurement of Microvascular Volumetric Flow Rates. Microvasc. Res. 1978, 15 (1), 93-101. https://doi.org/10.1016/0026-2862 (78) 90009-2.) Blood flow (Q) was calculated from the values measured as Q=π×V(D/2)\hat{ }2. Changes in arteriolar and venular diameter from baseline were used as indicators of a change in vascular tone. This calculation assumes a parabolic velocity profile and has been found to be applicable to tubes of 15-100 μm internal diameters and for Hets in the range of 6-60%. (Lipowsky, H. H.; Zweifach, B. W. Application of the “Two-Slit” Photometric Technique to the Measurement of Microvascular Volumetric Flow Rates. Microvasc. Res. 1978, 15 (1), 93-101. https://doi.org/10.1016/0026-2862 (78) 90009-2)

### Functional Capillary Density (FCD)

Functional capillaries, defined as capillary segments that have transit of at least one RBC in a 60 second period in 10 successive microscopic fields, were assessed in a region of 0.46 mm². The FCD (cm-1) was calculated as the total length of RBC perfused capillaries divided by the viewing area (0.46 mm²)

### Hypervolemic Infusion (Top-Load) Protocol

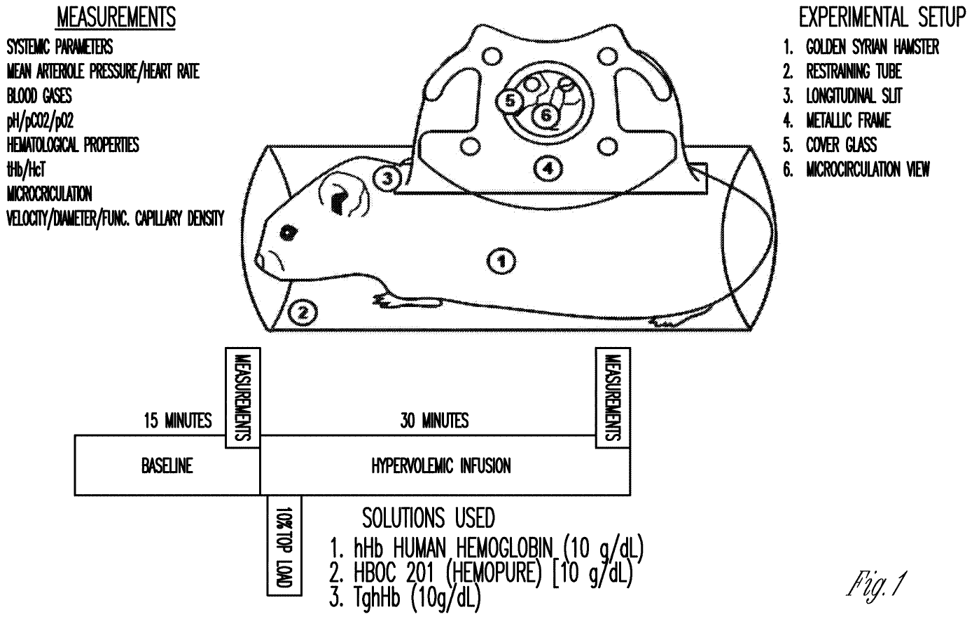

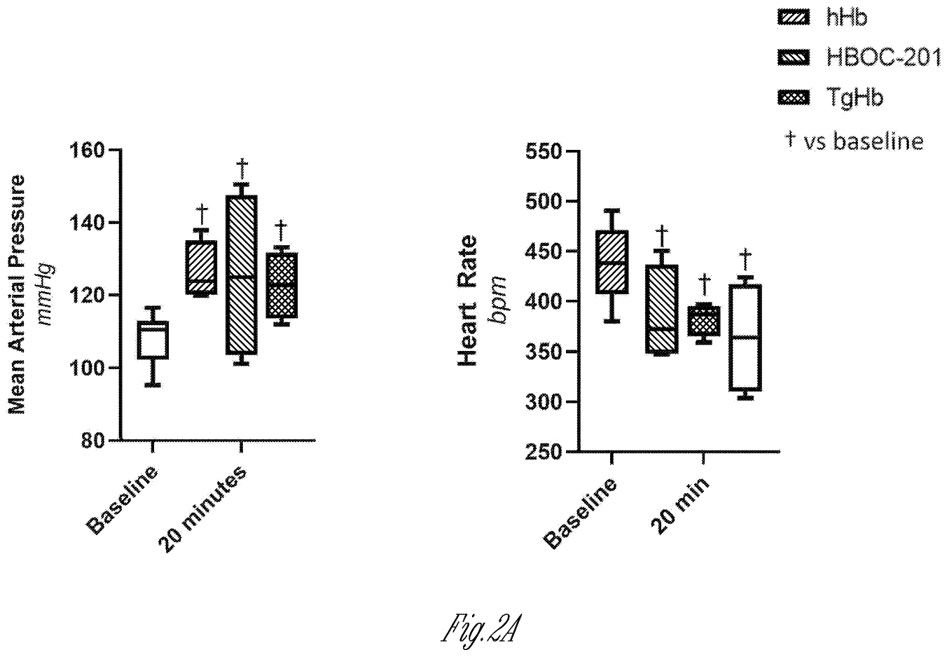

Hamsters received a hypervolemic infusion as a bolus through the carotid artery of 10% of the hamster's blood volume. The hamsters blood volume was calculated as 7% of their total body weight. The experimental groups were labeled based on the solution human hemoglobin (hHb), hemoglobin-based oxygen carrier-201 (HBOC-201), Hemopure®; Biopure Corporation, Cambridge, MA), TgHb all solutions were kept at a concentration of 100 mg/mL. After each infusion, animals were allowed 20 minutes to stabilize before systemic and microvascular characterization. The experimental timeline is described in FIG. 1.

### Pharmacokinetics and Oxidation Rate Measurements

Pharmacokinetics and oxidation rate was studied for 48 hours after exchange transfusion with 100 mg/mL TghHb. Briefly, hamsters fitted with the dorsal window chamber were exchanged with 40% of the estimated BV (7% of body weight). Test solutions were infused into the jugular vein catheter at a rate of 100 μl/min with simultaneous blood withdrawal at the same rate from the carotid artery catheter via a dual syringe pump (Harvard Apparatus, Holliston, MA). Blood samples (50 μL) were taken after exchange transfusion at 0.5, 1, 2, 8, 12, 24, and 48 hours. TghHb pharmacokinetic and oxidation rates were determined using non-compart-mental analysis.

Organ Harvesting and Analysis for Markers of Organ Damage and Inflammation.

Blood was collected and centrifuged to separate the plasma. Animals were sacrificed with Fatal Plus (sodium pentobarbital, 300 mg/kg), urine was collected, and heart, kidneys, liver, and spleen were harvested. Markers of inflammation, function, and organ injury were evaluated using the ELISA Kits/Assays described in Table 1.

Table 1 describes the ELISA Kits/Assays used to quantify markers of organ damage and inflammation.

TABLE 1

| Method | Kit/Assay | Analyte | Vendor |
|---|---|---|---|
| ELISA | KA1625 | AST | Abnova Corp, Taiwan |
| ELISA | KA4189 | ALT | Abnova Corp, Taiwan |
| ELISA | BMS625 | IL-6 | Thermo Fisher, Waltham, MA |
| ELISA | BMS629 | IL-10 | Thermo Fisher, Waltham, MA |
| ELISA | KB02-H2 | Creatinine | Arbor Assays Inc, Ann Arbor, MI |
| ELISA | K024-H5 | BUN | Arbor Assays Inc, Ann Arbor, MI |
| ELISA | ERCXCL1 | CXCL1 | Thermo Fisher, Waltham, MA |
| ELISA | MCA-155 | Ferritin | Serotec, Oxford, UK |
| ELISA | BA-E-8600 | Catecholamines | ImmunoSmol, France |
| ELISA | ERLON2 | Unne NGAL | Thermo Fisher, Waltham, MA |
| ELISA | ab235627 | Bilirubin | Abcem, Cambridge, UK |
| Luminex technology/ | RECYTNMAG- | MCP-1 | Millipore Corporation, |
| Miliplex Immunoassay | 66K | | Massachusetts, USA |
| Luminex technology/ | RECYTNMAG- | TNF-α | Millipore Corporation, |
| Miliplex Immunoassay | 65K | | Massachusetts, USA |
| ELISA | ab246529 | Cardiac Troponin | Abcam, Cambridge, UK |
| ELISA | Ab256398 | CRP | Abcam, Cambridge, UK |
| ELISA | Ab108797 | ANP | Abcam, Cambridge, UK |

### Statistical Analysis

Results are presented as mean±standard deviation. Data within each group was analyzed using analysis of variance for repeated measurements (ANOVA, Kruskal-Wallis test). Microhemodynamic measurements were compared to baseline levels obtained before the experimental procedure. The box-whisker plot separates the data into quartiles, with the top of the box defining the 75th percentile, the line within the box giving the median, and the bottom of the box showing the 25th percentile. The upper “whisker” defines the 95^{th} percentile and the lower whisker defines the 5^{th} percentile. Microhemodynamic data is presented as absolute values and ratios relative to baseline values. A ratio of 1.0 signifies no change from baseline while lower and higher ratios are indicative of changes proportionally lower and higher than baseline (i.e., 1.5 would mean a 50% increase from the baseline level). The same vessels and capillary fields were followed so that direct comparisons to their baseline levels could be performed, allowing for more robust statistics for small sample populations. All statistics were calculated using GraphPad Prism 4.01 (GraphPad Software, Inc., San Diego, CA). Changes were considered statistically significant if p<0.05

### Results

### Mean Arterial Pressure (MAP), Heart Rate (HR), Hematological Properties and Blood Gases

Table 2 represents the baseline values and the measured quantities of total hemoglobin concentration (tHb), hematocrit (Hct), mean arterial pressure (MAP), heart rate (HR), pH, pCO₂, pO₂, after the hypervolemic infusion of HBOC-201, human hemoglobin (hHb), transglutaminase polymerized human hemoglobin (TgHb).

TABLE 2
20 Minutes After Hypovolemic Infusion

| | Baseline | hHb | HBOC-201 | TgHb |
|---|---|---|---|---|
| tHb (g/dl) | 15.5 ± 0.4 | 15.0 ± 0.9 | 15.2 ± 0.9 | 15.4 ± 0.3 |
| Hct (%) | 47 ± 0.6 | 44 ± 4 | 44 ± 3 | 45 ± 1 |
| MAP (mmHg) | 108.5 ± 13.6 | 126.3 ± 8.2^{†} | 121.0 ± 23.1^{†} | 122.6 ± 9.4^{†} |
| HR (bpm) | 430.12 ± 19.38 | 393.1 ± 65.29^{†} | 352.50 ± 57.27^{†} | 341.3 ± 56.29^{†} |
| pH | 7.36 ± 0.05 | 7.35 ± 0.02 | 7.34 ± 0.11 | 7.36 ± 0.05 |
| pCO₂ (mmHg) | 58.0 ± 6.8 | 56.0 ± 4.8 | 55.8 ± 8.3 | 55.6 ± 8.6 |
| pO₂ (mmHg) | 61.3 ± 11.22 | 58.7 ± 7.0 | 69.4% 17.4 | 57.3 ± 16.3 |
| pHb (g/dL) | — | 1.1 ± 0.3 | 1.0± 0.1 | 1.0 ± 0.2 |

^{†}vs baseline

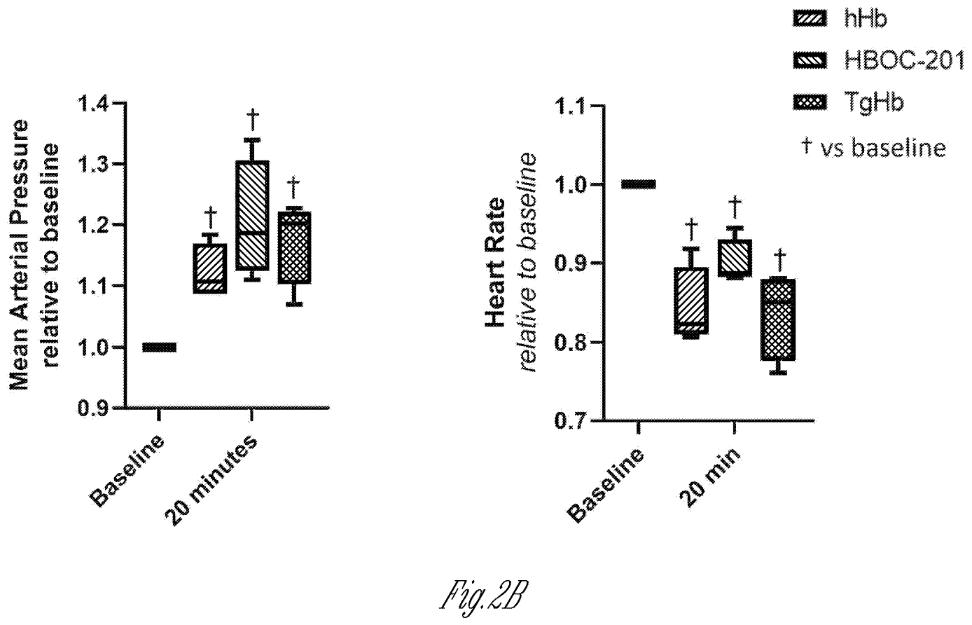

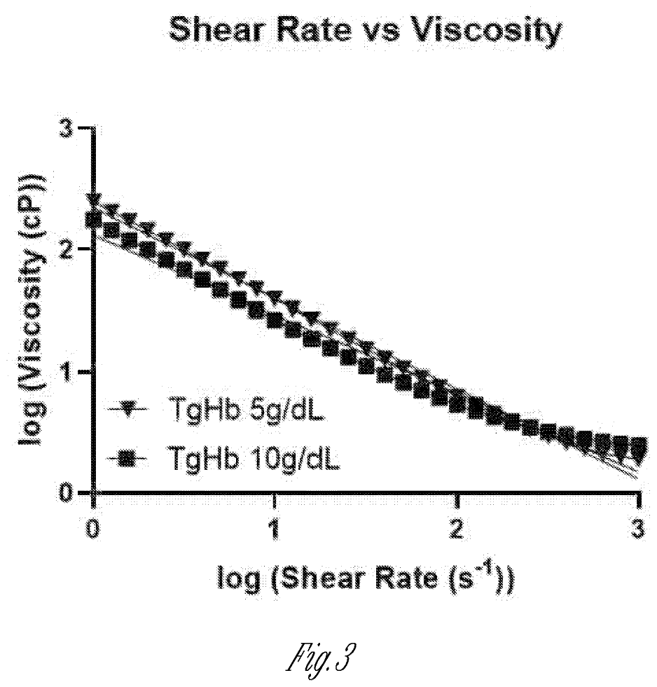

FIG. 2 indicates results of MAP and HR with their absolute values and their values relative to baseline. MAP measurements were significant to baseline following the hypervolemic infusion of hHb, HBOC-201, and TgHb. HR measurements were significant to baseline following the hypervolemic infusion of hHb, HBOC-201, and TgHb.

Total hemoglobin concentration (tHb), hematocrit (Hct), pH, pCO2, and pO2 showed no significance to baseline following the hypervolemic infusion of hHb, HBOC-201, and TgHb. There was no significance between groups following the hypervolemic infusion of hHb, HBOC-201, and TgHb.

Table 3 represents the colloid osmotic pressure of, hHb, HBOC-201 and TgHb (at a concetration of 10 g/dL).

TABLE 3
Colloid Osmotic Pressure

| | hHb | HBOC-201 | TgHb |
|---|---|---|---|
| COP (mmHg) | 50 | 40* | 32 |

*HBOC-201 was recovered from literature values at a concentration of 13.2 g/dL

### Example 3: Biophysical Properties

Table 4A indicates the values of the viscosity and the shear stress of a solution containing 5 g/dL of TgHb at shear rates from 0 to 1000.

TABLE 4A

| Shear rate | Viscosity | Step time | Normal stress | Stress | Temperature | Viscosity |
|---|---|---|---|---|---|---|
| (1/s) | (cP) | (s) | (Pa) | (Pa) | (° C.) | (Pa · s) |
| 999.999 | 1.90207 | 21.7632 | −306.233 | 1.90206 | 37.016 | 0.001902 |
| 794.328 | 1.99976 | 47.8422 | −278.877 | 1.58847 | 37.006 | 0.002 |
| 630.958 | 2.13687 | 73.9508 | −262.832 | 1.34828 | 37 | 0.002137 |
| 501.187 | 2.33643 | 100.106 | −253.207 | 1.17099 | 37.018 | 0.002336 |
| 398.107 | 2.59948 | 126.309 | −245.213 | 1.03487 | 37.004 | 0.002599 |
| 316.227 | 2.93496 | 152.502 | −241.137 | 0.928114 | 37.008 | 0.002935 |
| 251.189 | 3.35409 | 178.644 | −237.949 | 0.842509 | 36.982 | 0.003354 |
| 199.526 | 3.87405 | 204.766 | −237.511 | 0.772974 | 37.007 | 0.003874 |
| 158.489 | 4.51169 | 230.925 | −235.438 | 0.715055 | 36.997 | 0.004512 |
| 125.893 | 5.29537 | 257.089 | −234.98 | 0.666648 | 37.001 | 0.005295 |
| 99.9997 | 6.26126 | 283.23 | −233.411 | 0.626124 | 36.991 | 0.006261 |
| 79.4328 | 7.43577 | 309.429 | −233.216 | 0.590644 | 36.992 | 0.007436 |
| 63.0958 | 8.89134 | 335.551 | −232.581 | 0.561007 | 37.001 | 0.008891 |
| 50.1186 | 10.6587 | 361.716 | −231.695 | 0.5342 | 37.001 | 0.010659 |
| 39.8106 | 12.8017 | 382.839 | −232.002 | 0.509642 | 37.002 | 0.012802 |
| 31.6228 | 15.418 | 403.999 | −232.319 | 0.46756 | 36.998 | 0.015418 |
| 25.1188 | 18.581 | 425.078 | −231.731 | 0.466733 | 37.005 | 0.018581 |
| 19.9526 | 22.4556 | 446.251 | −231.201 | 0.448049 | 37 | 0.022456 |
| 15.8491 | 27.1234 | 467.435 | −230.887 | 0.429882 | 37 | 0.027123 |
| 12.5892 | 32.6617 | 488.594 | −229.944 | 0.411183 | 37.01 | 0.032662 |
| 9.99984 | 39.2733 | 509.736 | −231.16 | 0.392726 | 37 | 0.039273 |
| 7.94332 | 47.3149 | 530.854 | −231.659 | 0.375837 | 37.019 | 0.047315 |
| 6.3096 | 57.078 | 551.945 | −230.806 | 0.360139 | 37.005 | 0.057078 |
| 5.01186 | 68.9065 | 573.041 | −230.087 | 0.34535 | 37.004 | 0.068907 |
| 3.98119 | 82.6558 | 594.123 | −230.395 | 0.329068 | 37.001 | 0.082656 |
| 3.16246 | 99.7677 | 615.284 | −230.557 | 0.315512 | 37.007 | 0.099768 |
| 2.51199 | 120.04 | 636.499 | −228.866 | 0.30154 | 37.004 | 0.12004 |
| 1.99524 | 143.946 | 657.664 | −227.937 | 0.287208 | 37.004 | 0.143946 |
| 1.58497 | 172.077 | 678.726 | −230.458 | 0.272736 | 37.001 | 0.172077 |
| 1.25907 | 205.959 | 699.828 | −230.47 | 0.259316 | 36.99 | 0.205959 |
| 0.999953 | 248.59 | 720.976 | −227.778 | 0.248579 | 37.001 | 0.24859 |

Table 4B indicates the values of the viscosity and the shear stress of a solution containing 10 g/dL TgHb at shear rates from 0 to 1000.

TABLE 4B

| Shear rate | Viscosity | Step time | Normal stress | Stress | Temperature | Viscosity |
|---|---|---|---|---|---|---|
| (1/s) | (cP) | (sec) | (Pa) | (Pa) | (° C.) | (Pa · s) |
| 999.999 | 2.47088 | 26.1348 | −237.266 | 2.47067 | 37.002 | 0.002471 |
| 794.328 | 2.54577 | 52.666 | −217.792 | 2.02218 | 37.017 | 0.002546 |
| 630.957 | 2.65516 | 78.7607 | −205.582 | 1.67529 | 36.997 | 0.002655 |
| 501.188 | 2.80169 | 104.902 | −197.832 | 1.40417 | 37.01 | 0.002802 |
| 398.107 | 2.9885 | 131.085 | −192.567 | 1.18974 | 37 | 0.002969 |
| 316.229 | 3.21349 | 157.264 | −191.763 | 1.0162 | 37.002 | 0.003213 |
| 251.189 | 3.48977 | 183.375 | −190.464 | 0.876591 | 37 | 0.00349 |
| 199.526 | 3.62562 | 209.53 | −191.558 | 0.763312 | 36.994 | 0.003826 |
| 158.489 | 4.23784 | 235.673 | −192.076 | 0.671653 | 37.013 | 0.004238 |
| 125.893 | 4.74361 | 261.815 | −193.699 | 0.597185 | 36.995 | 0.004744 |
| 99.9998 | 5.36326 | 287.941 | −193.957 | 0.536325 | 36.996 | 0.005363 |
| 79.4328 | 6.12155 | 314.046 | −195.122 | 0.486252 | 37.006 | 0.006122 |
| 63.0959 | 7.04003 | 340.18 | −196.006 | 0.444197 | 37.002 | 0.00704 |
| 50.1185 | 8.15168 | 361.344 | −197.756 | 0.40856 | 37.01 | 0.008152 |
| 39.8106 | 9.5213 | 382.454 | −198.78 | 0.379049 | 36.993 | 0.009521 |
| 31.6227 | 11.2005 | 408.522 | −200.143 | 0.354189 | 37.008 | 0.011201 |
| 25.1188 | 13.2105 | 429.633 | −201.011 | 0.331832 | 36.989 | 0.013211 |
| 19.9527 | 15.6879 | 450.75 | −202.863 | 0.313017 | 37.01 | 0.015688 |
| 15.8488 | 18.7423 | 471.907 | −204.359 | 0.297043 | 37.001 | 0.018742 |
| 12.5892 | 22.4175 | 492.989 | −204.396 | 0.282217 | 36.995 | 0.022418 |
| 10.0001 | 26.793 | 514.055 | −206.503 | 0.267934 | 36.997 | 0.026793 |
| 7.94301 | 32.2686 | 535.911 | −207.744 | 0.25631 | 36.994 | 0.032269 |
| 6.3097 | 38.8599 | 557.029 | −206.706 | 0.245194 | 37.005 | 0.03886 |
| 5.01174 | 46.9261 | 578.133 | −207.865 | 0.235192 | 37.001 | 0.046926 |
| 3.98107 | 56.6405 | 599.3 | −208.24 | 0.22549 | 37.005 | 0.056641 |
| 3.16202 | 66.2944 | 620.453 | −209.354 | 0.215949 | 37 | 0.066294 |
| 2.512 | 82.9499 | 641.567 | −210.763 | 0.20837 | 37.003 | 0.08295 |
| 1.99498 | 99.7694 | 662.662 | −210.046 | 0.199078 | 37.006 | 0.099769 |
| 1.5849 | 120.021 | 683.743 | −212.076 | 0.190221 | 37.006 | 0.120021 |
| 1.25888 | 144.976 | 704.815 | −213.719 | 0.162507 | 37.015 | 0.144976 |
| 0.999996 | 175.799 | 725.894 | −213.011 | 0.175781 | 37.007 | 0.175799 |

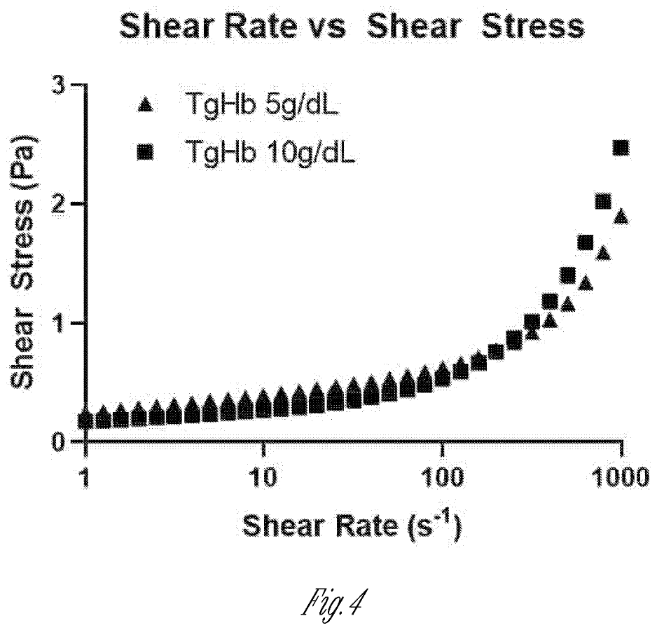

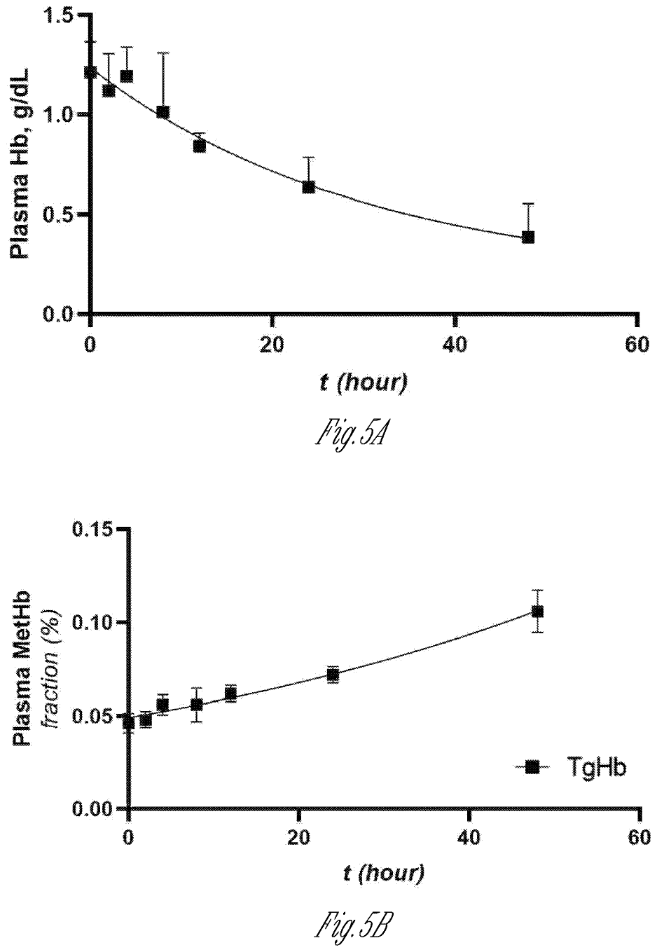

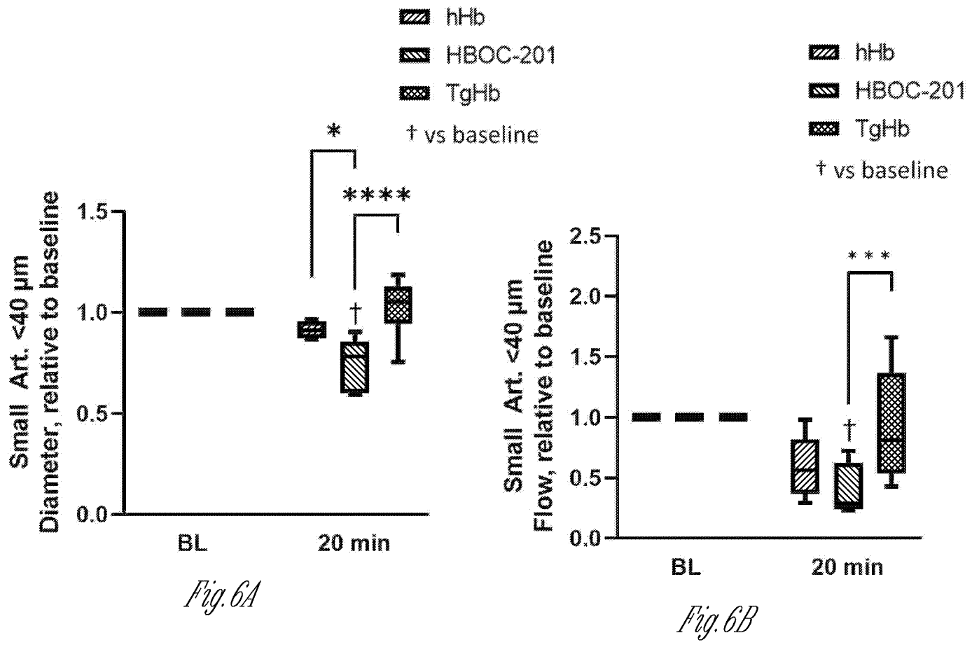

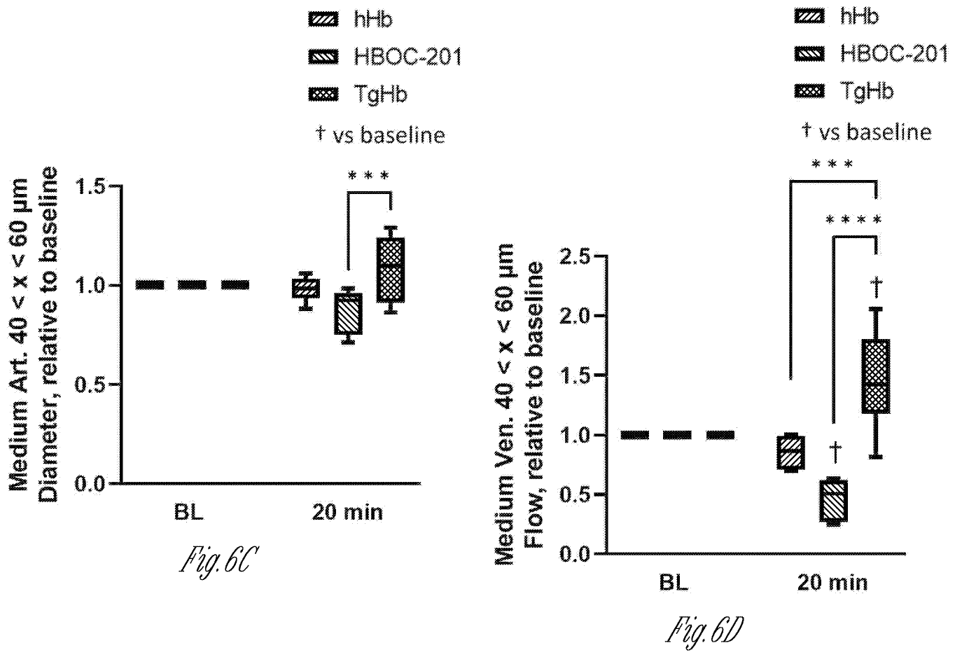

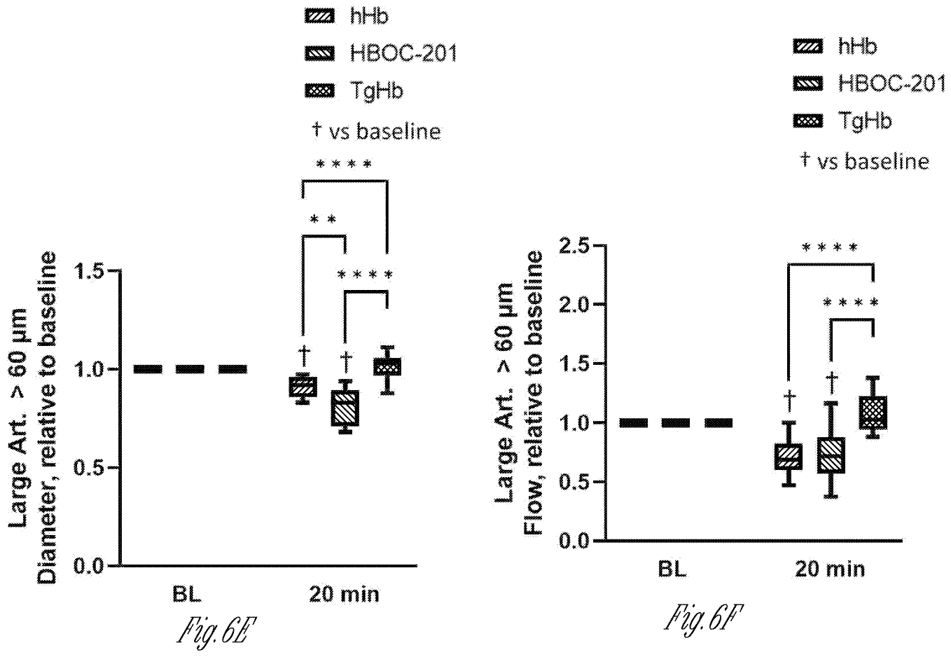

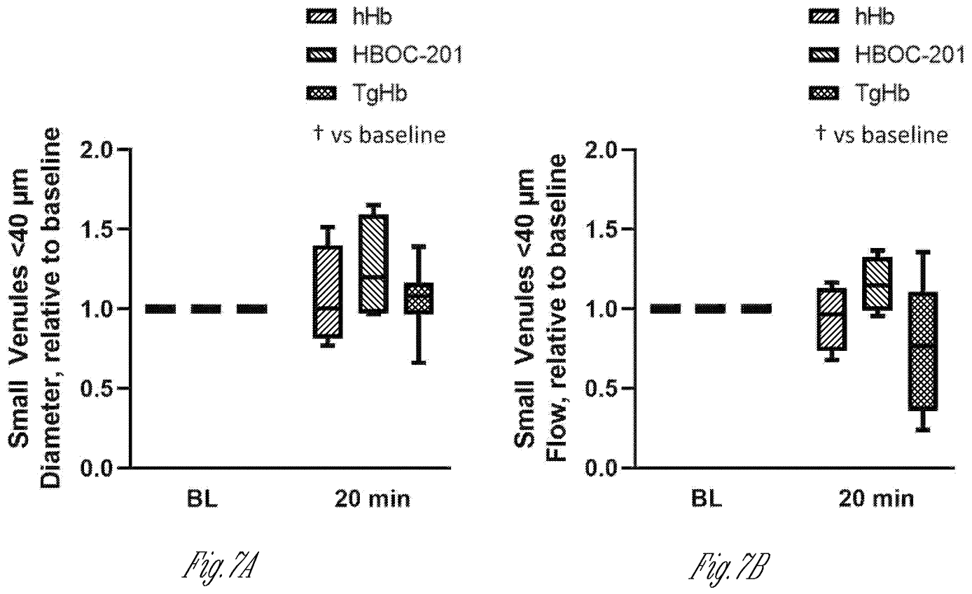

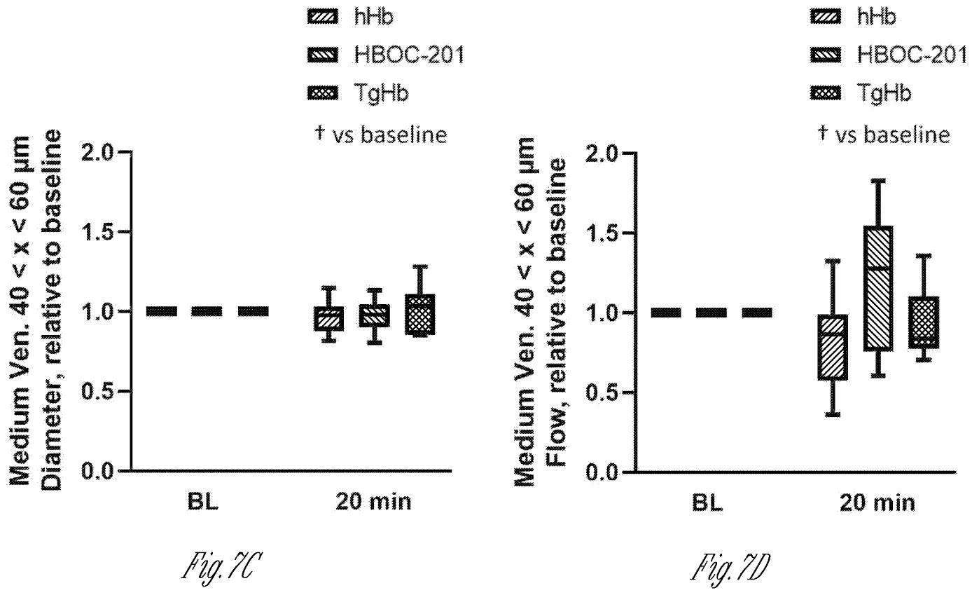

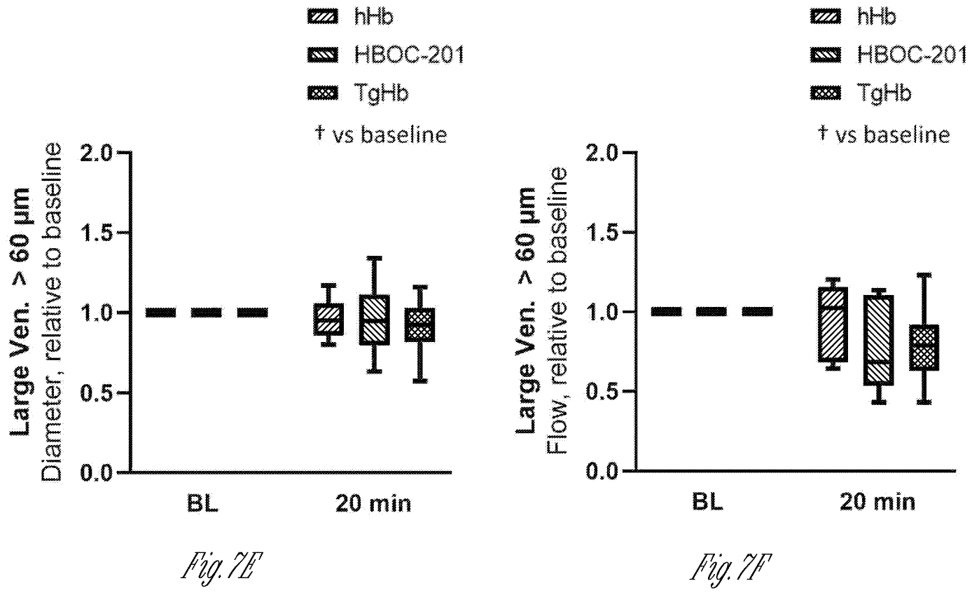

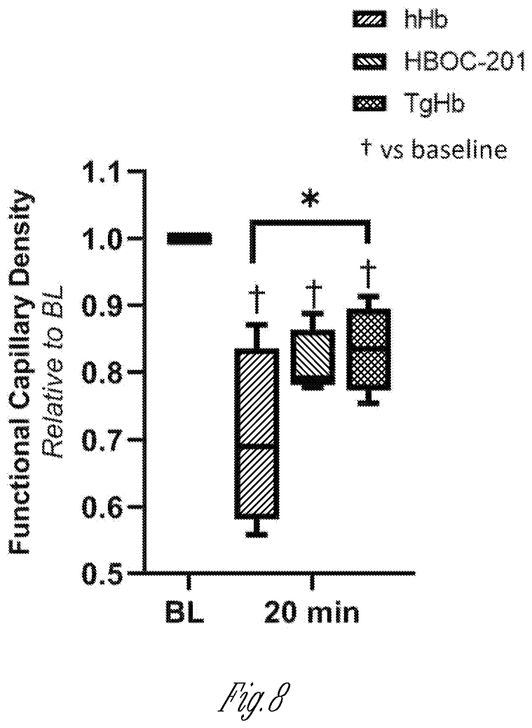

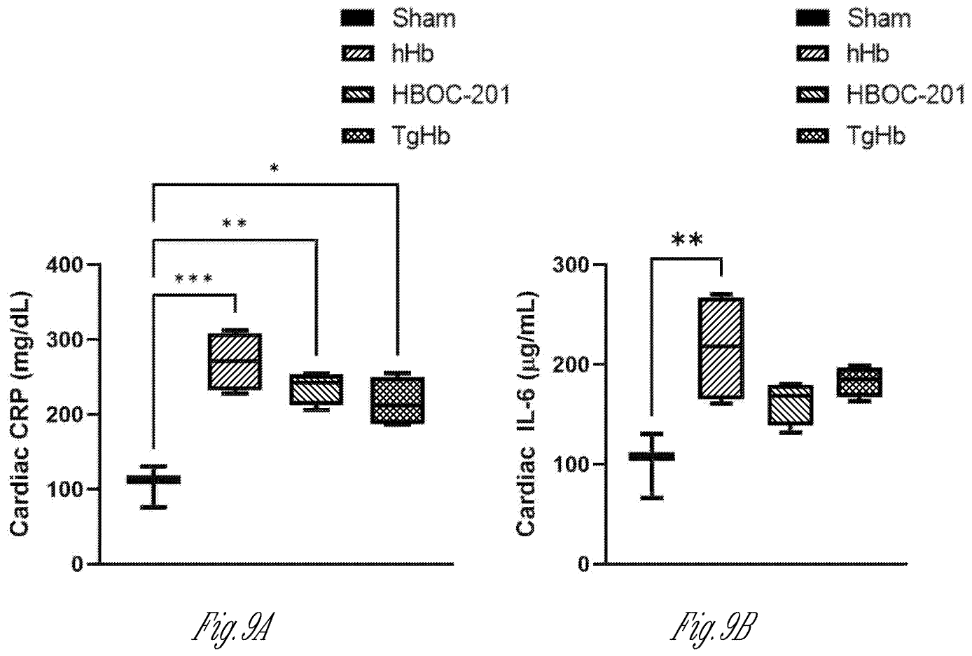

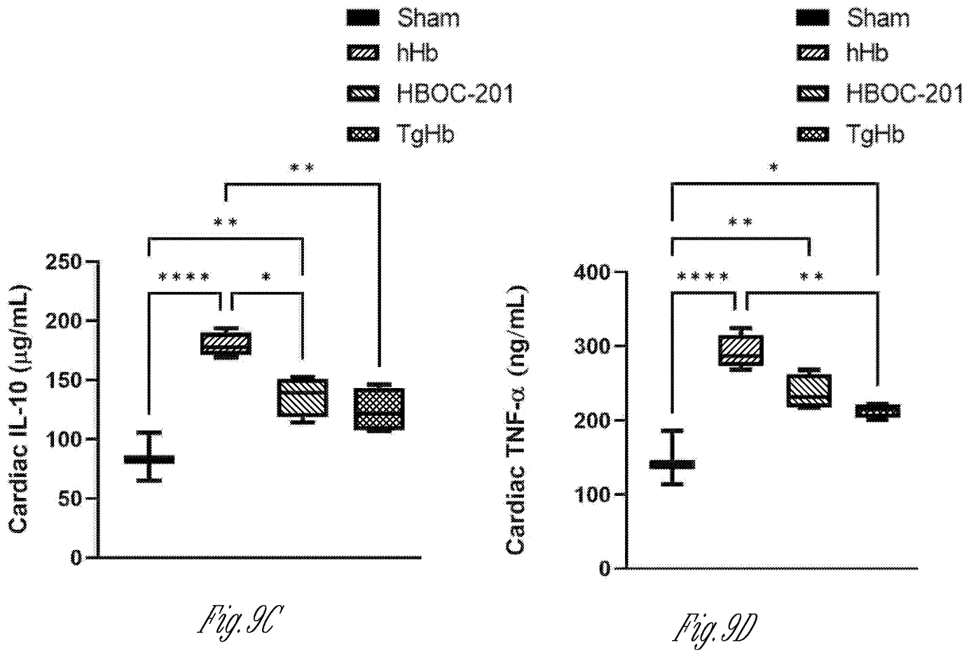

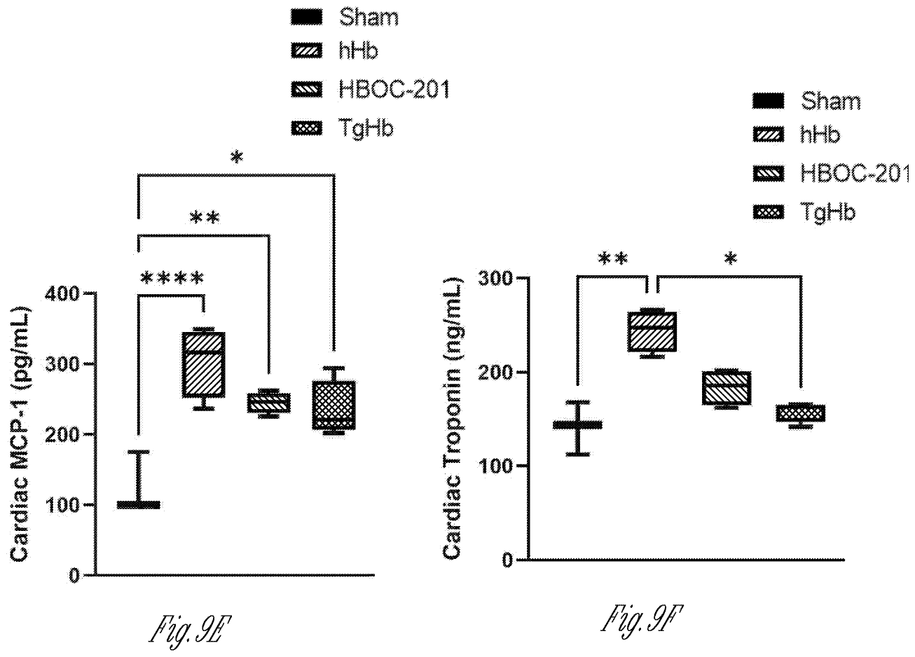

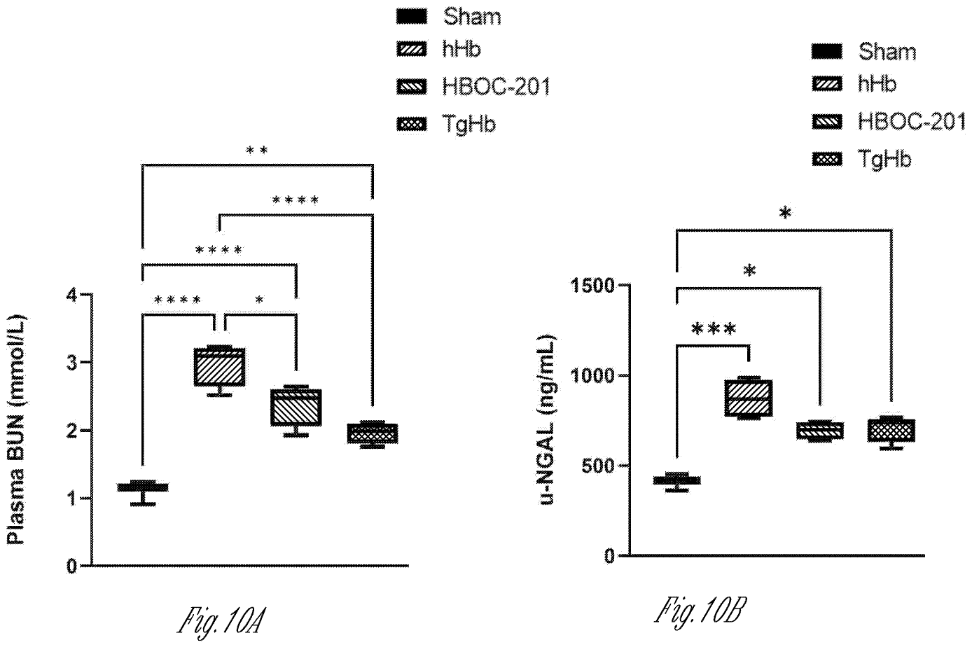

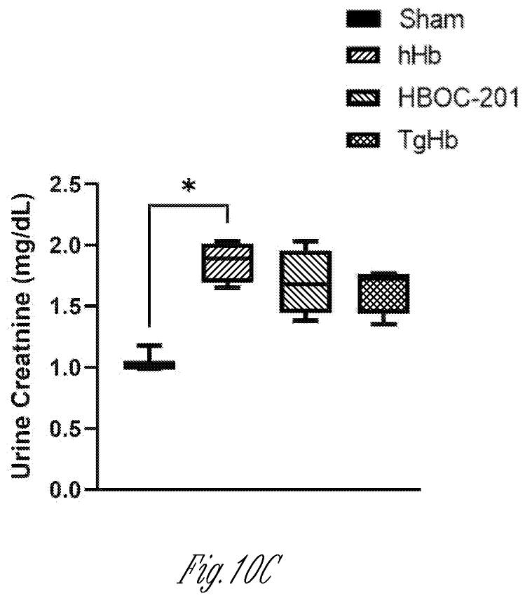

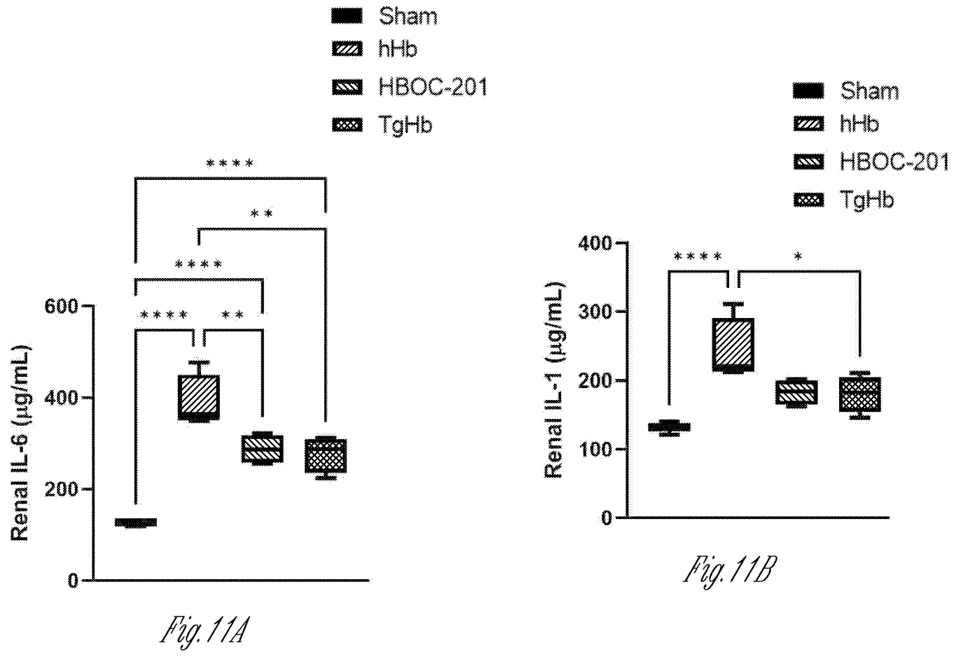

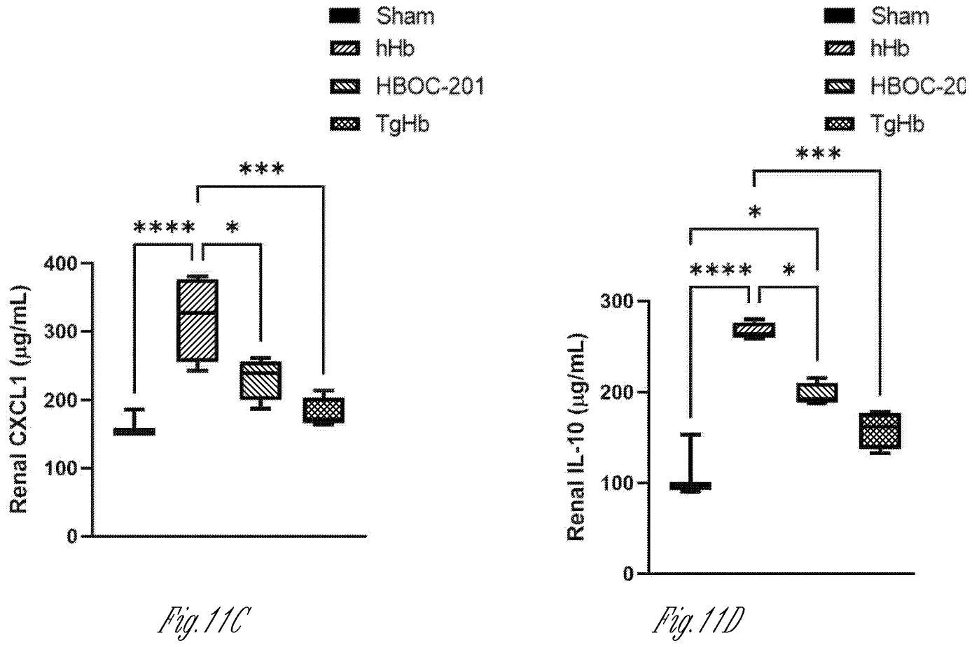

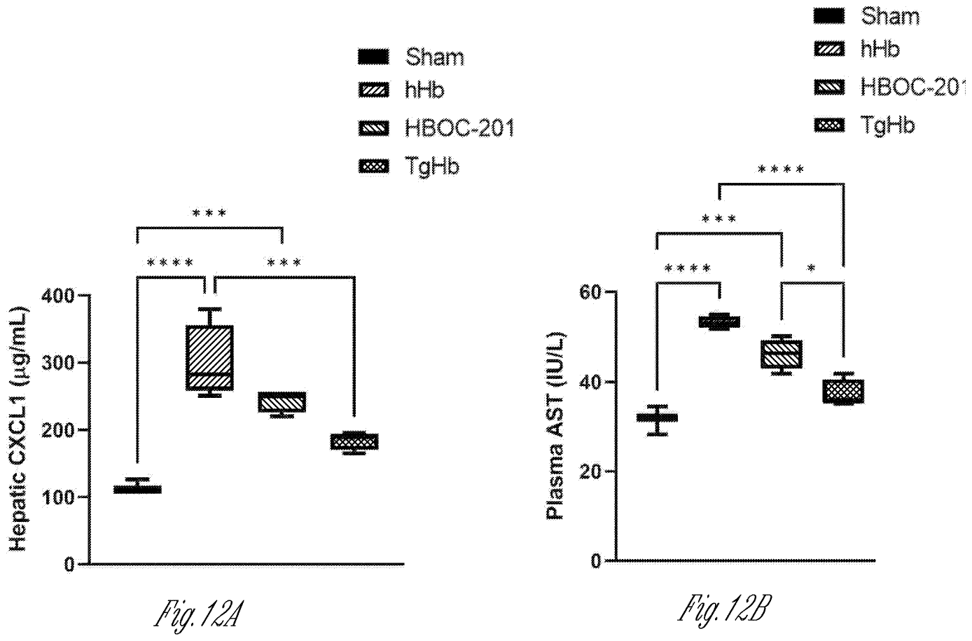

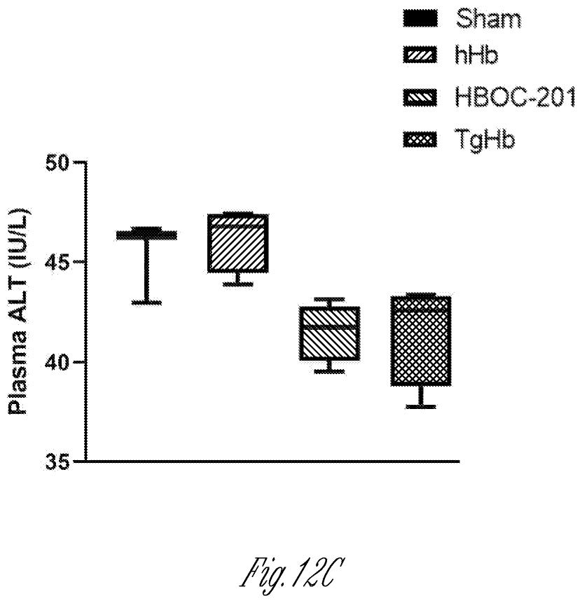

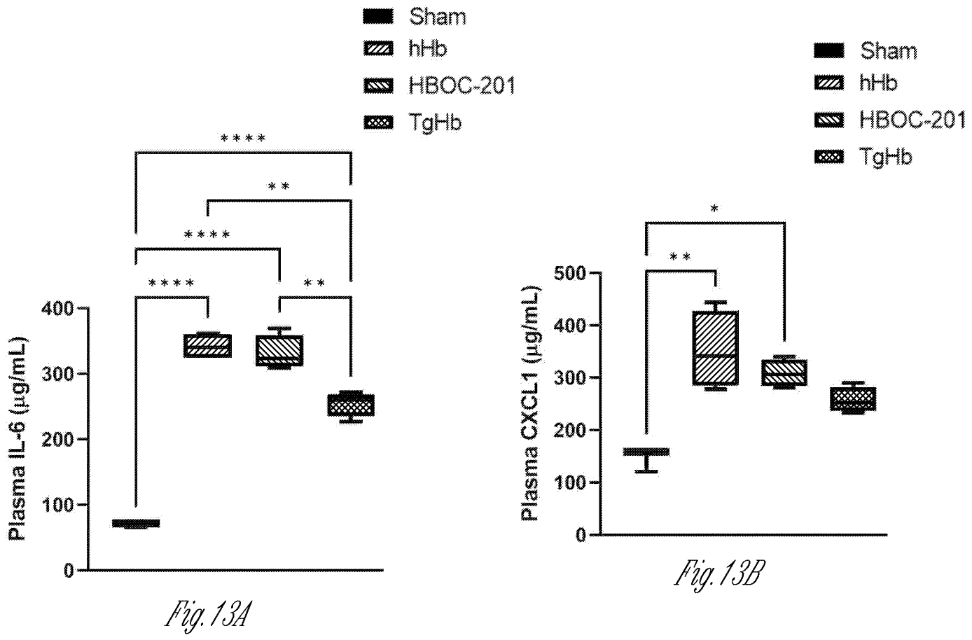

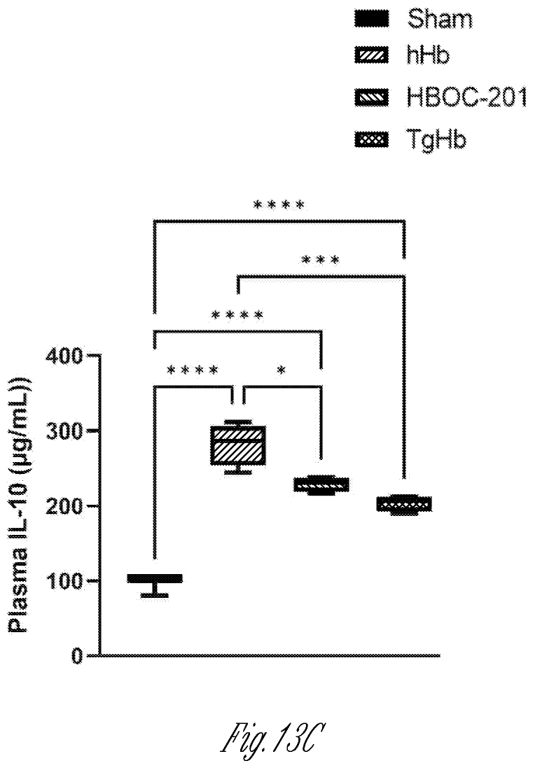

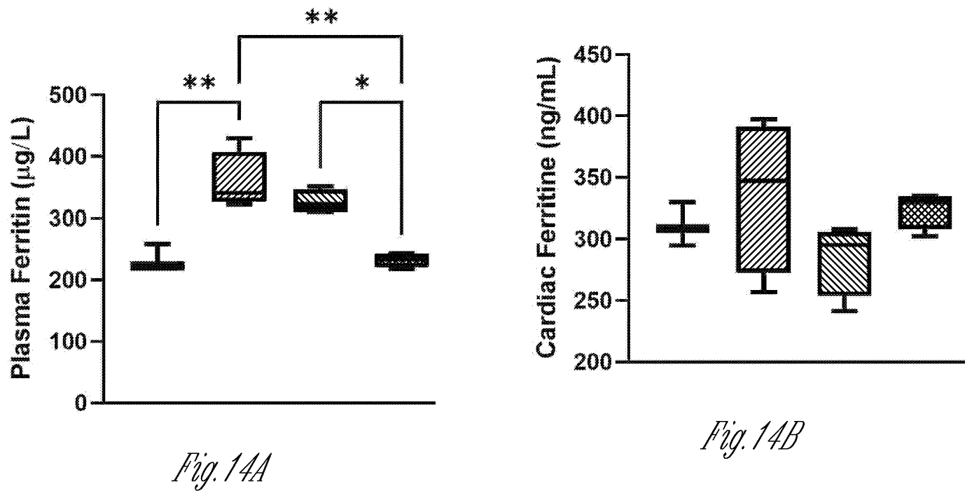

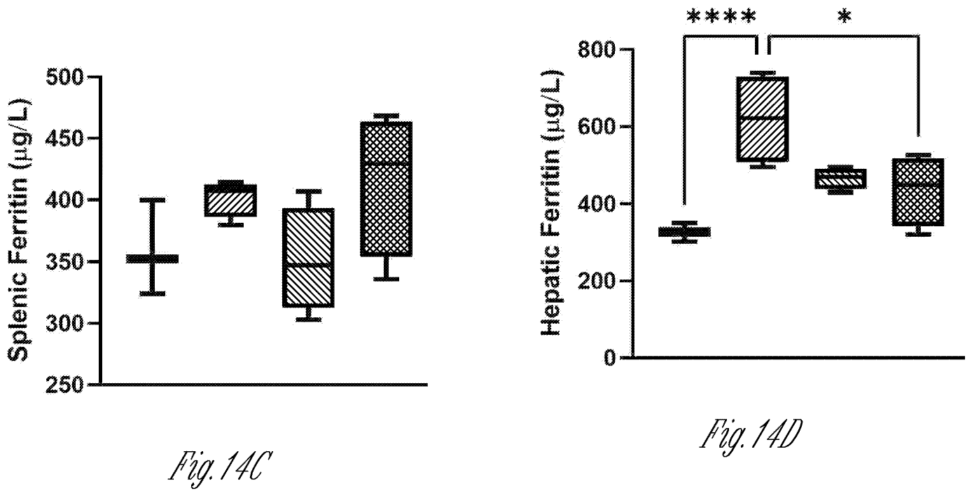

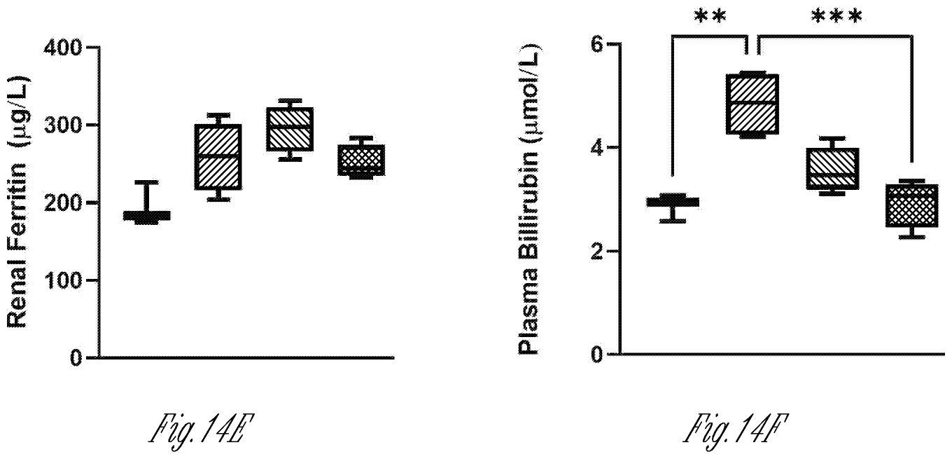

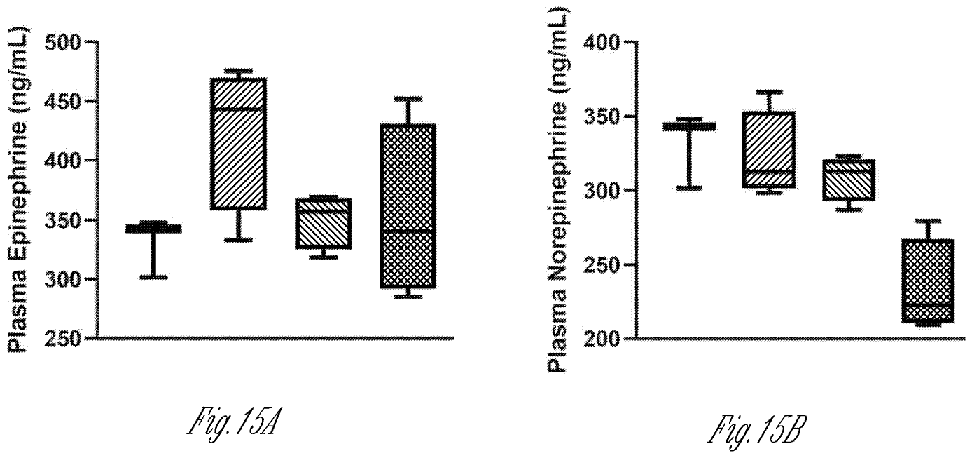

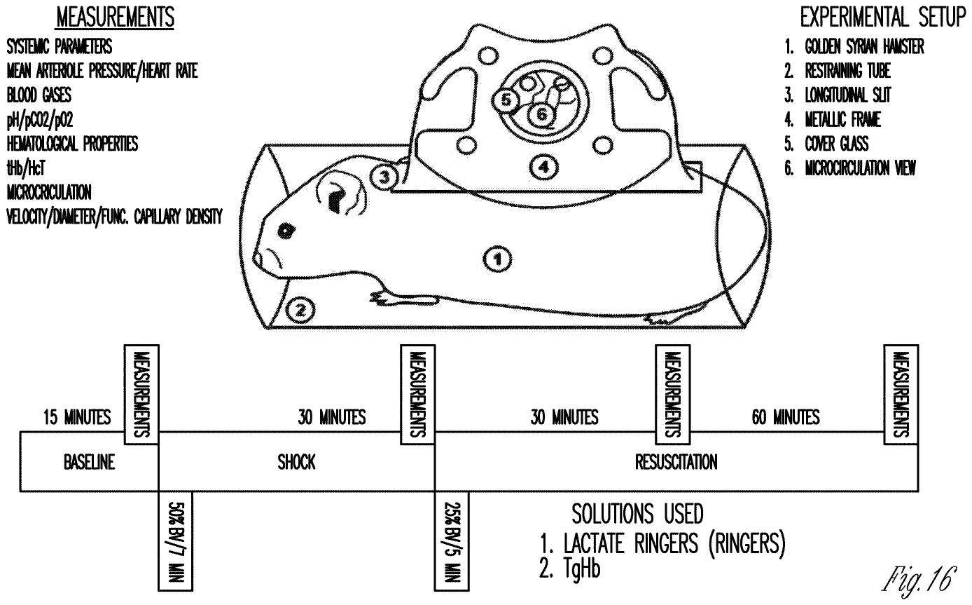

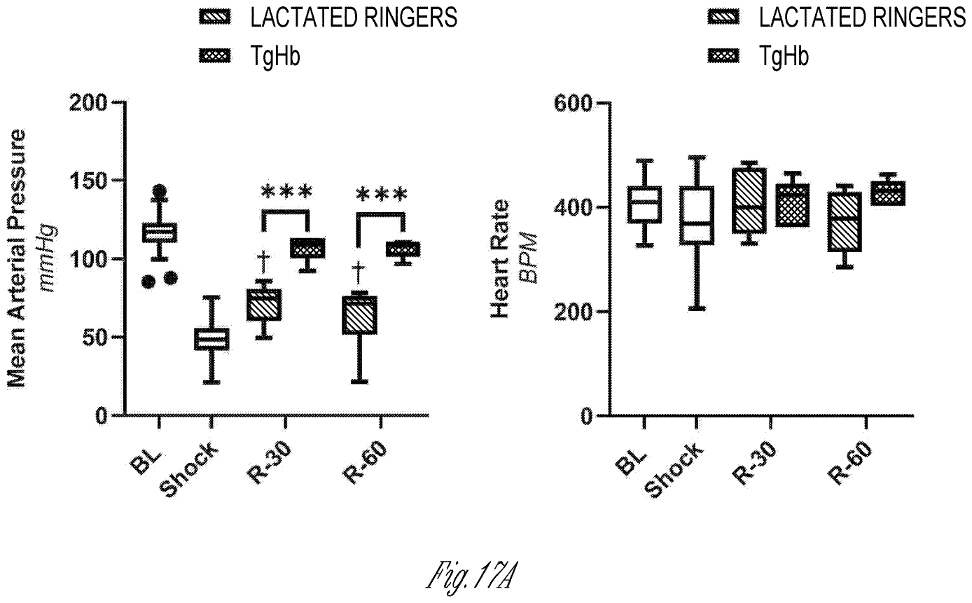

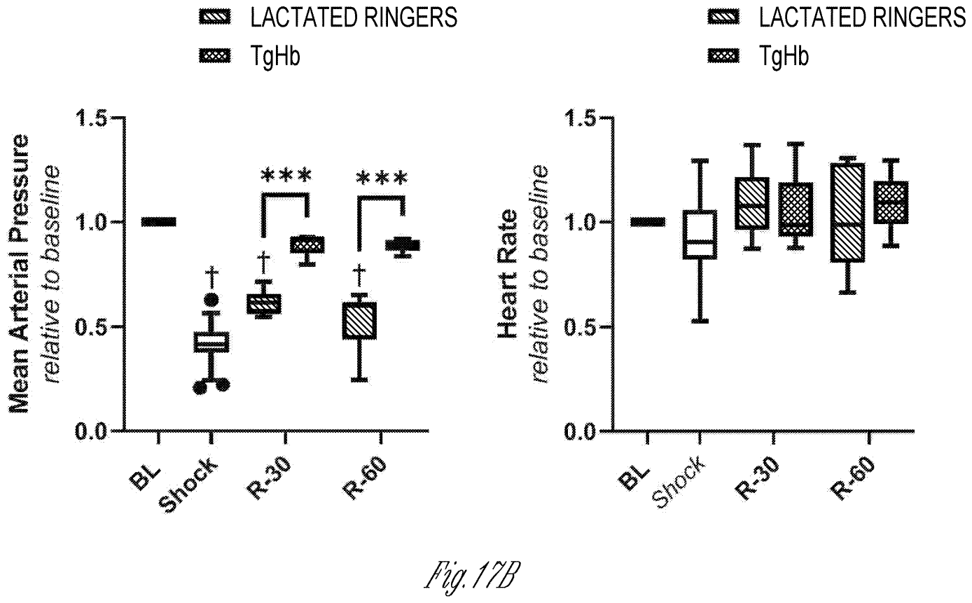

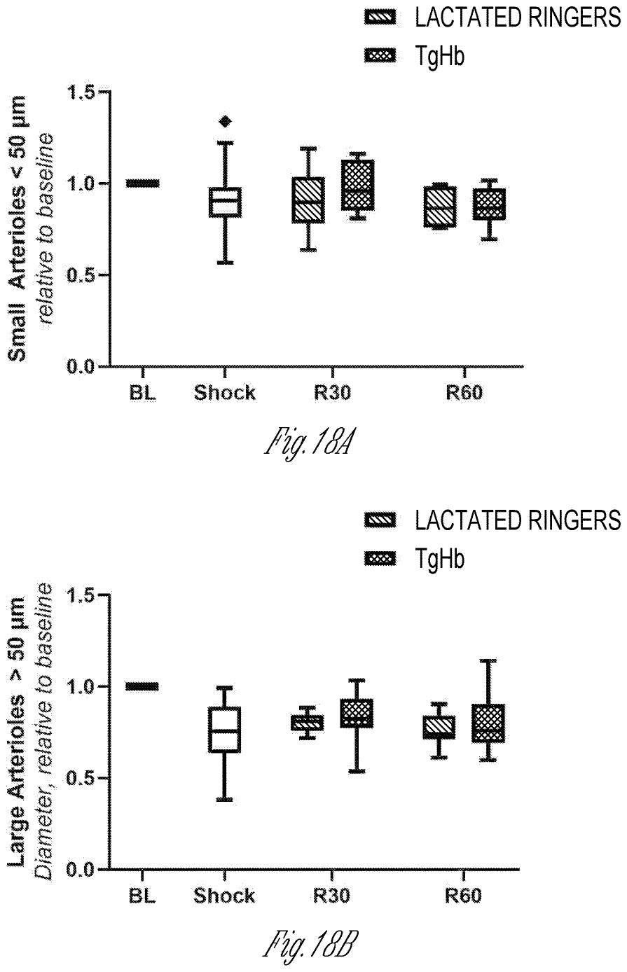

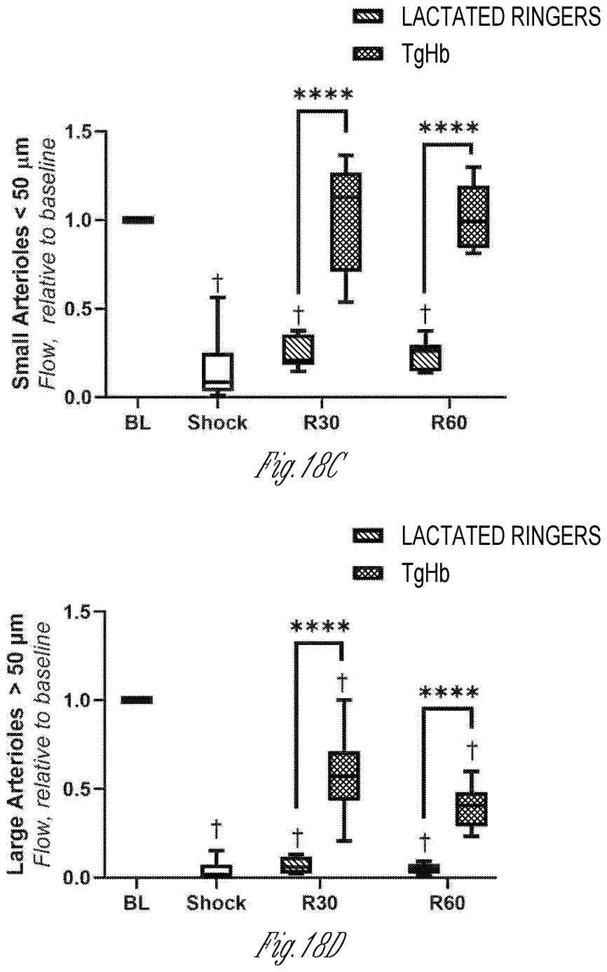

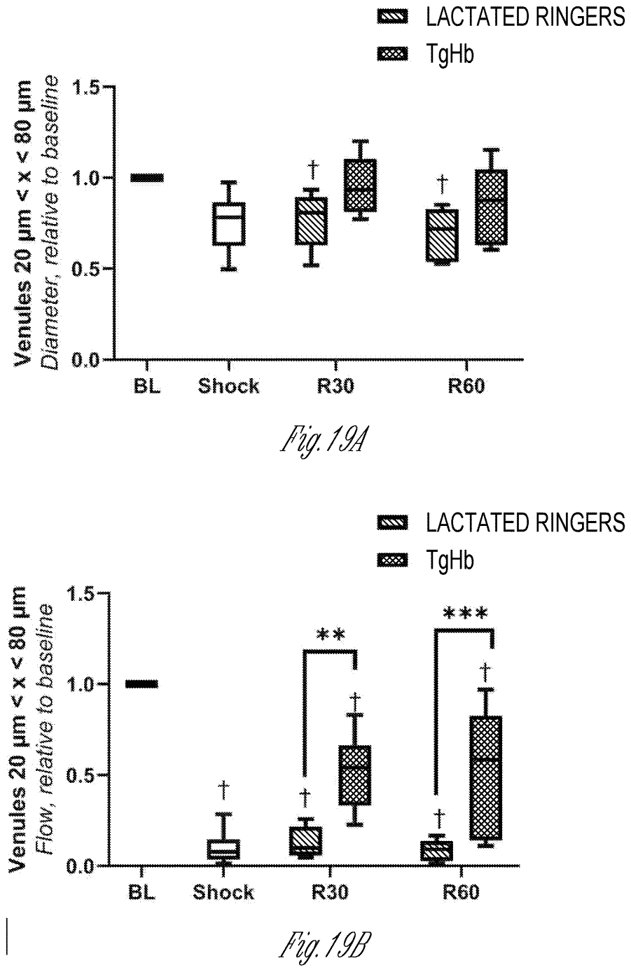

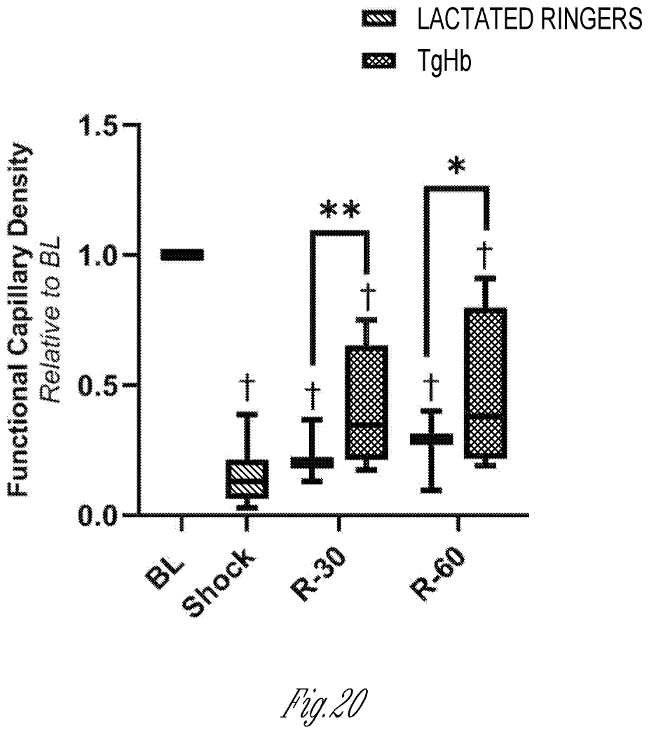

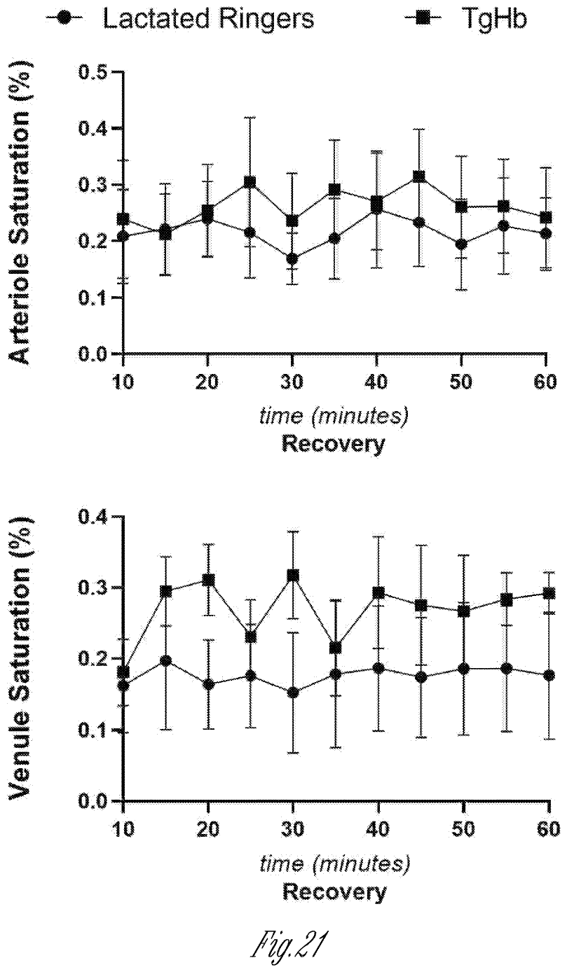

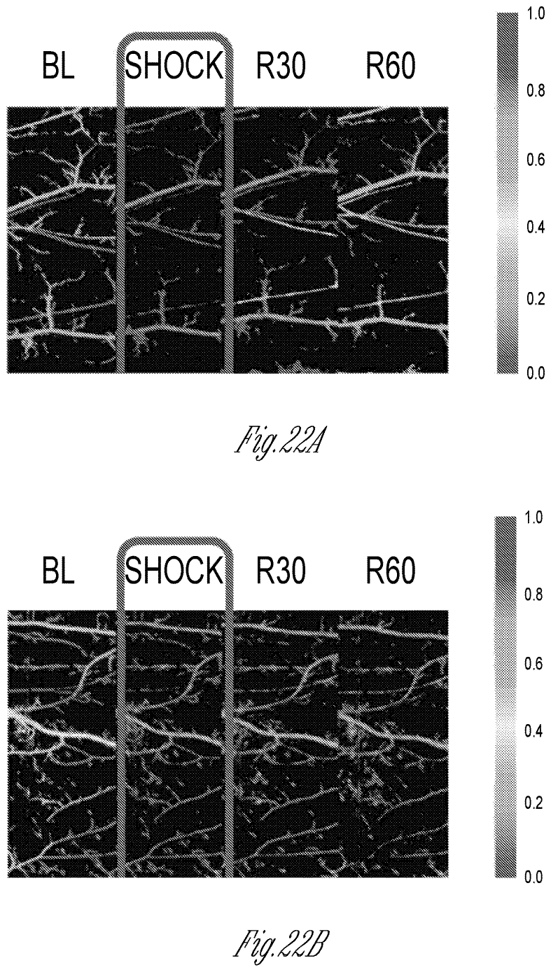

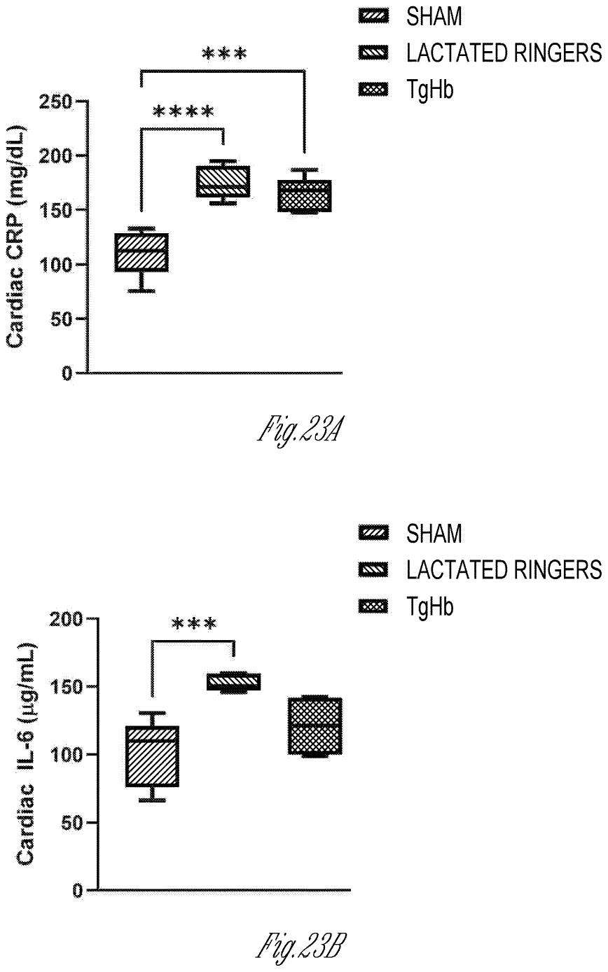

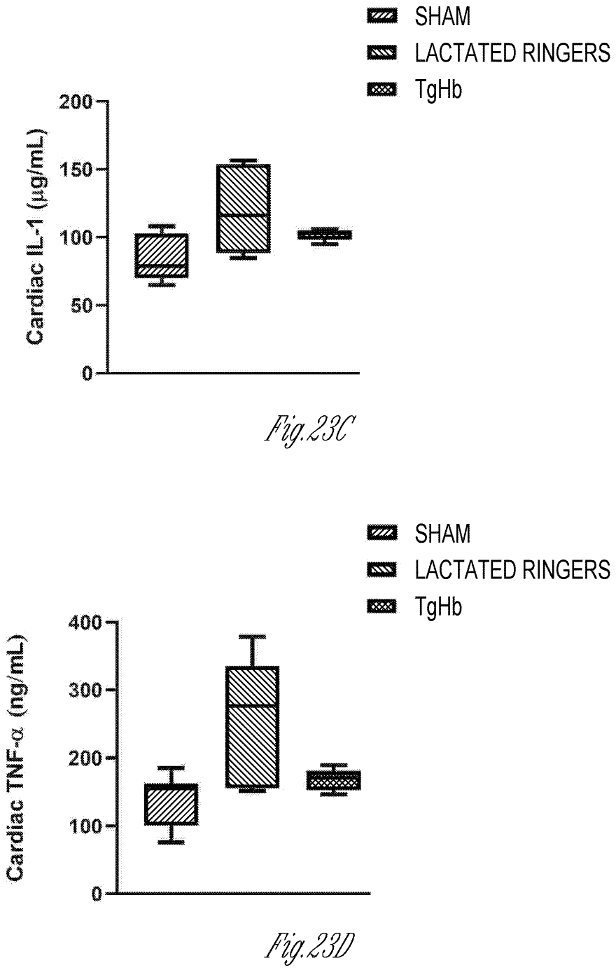

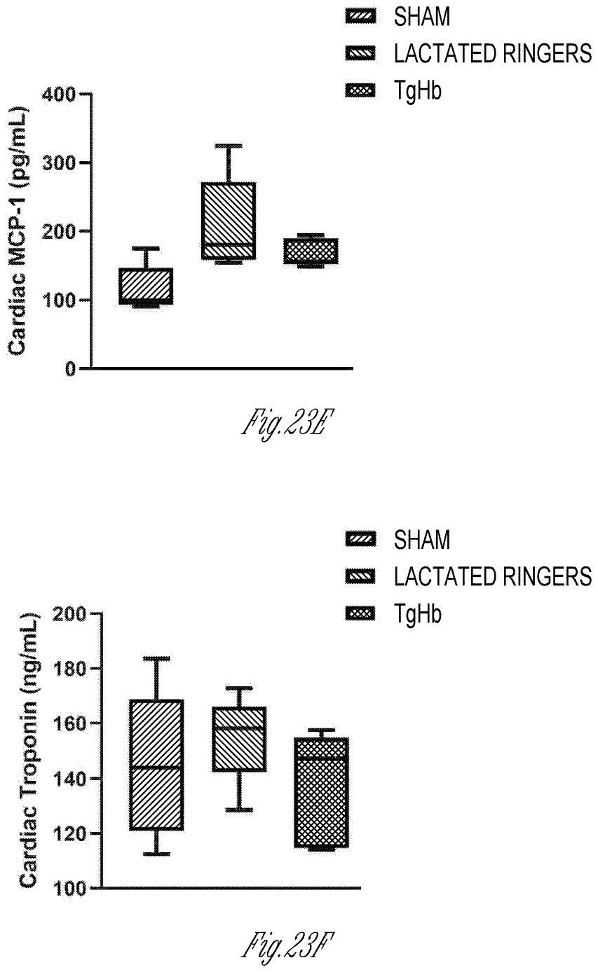

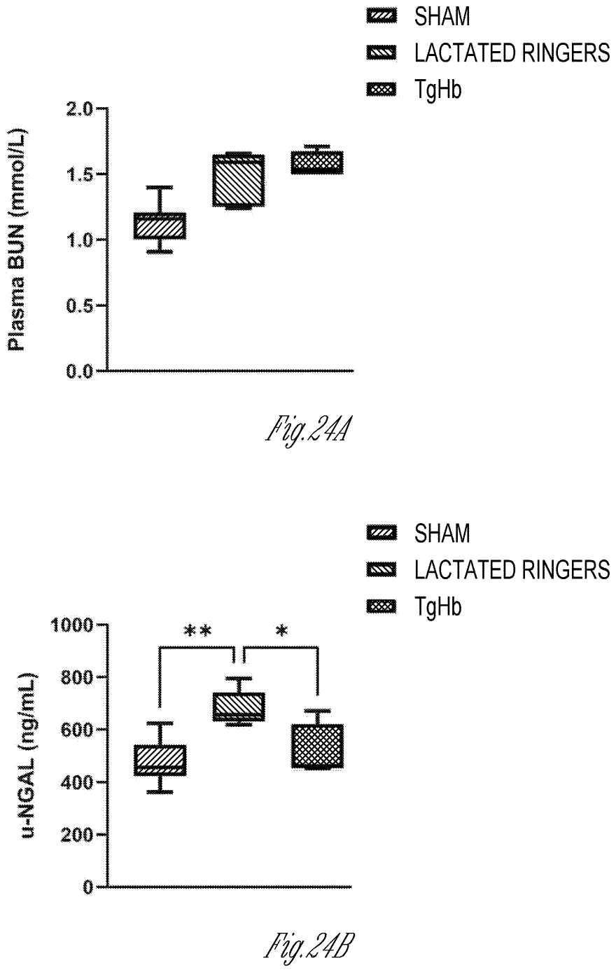

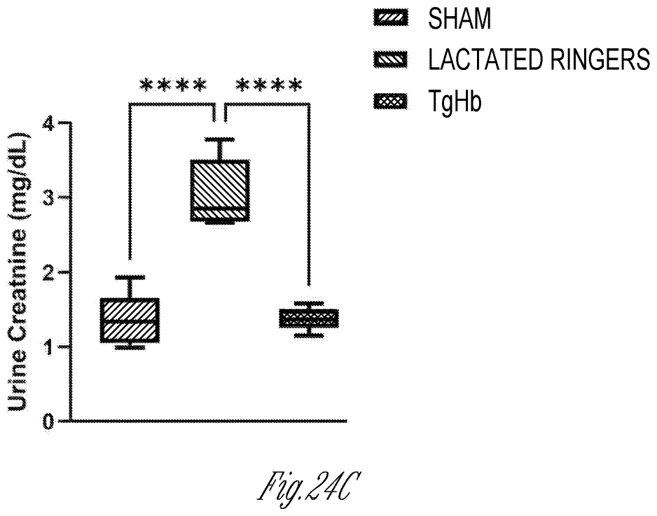

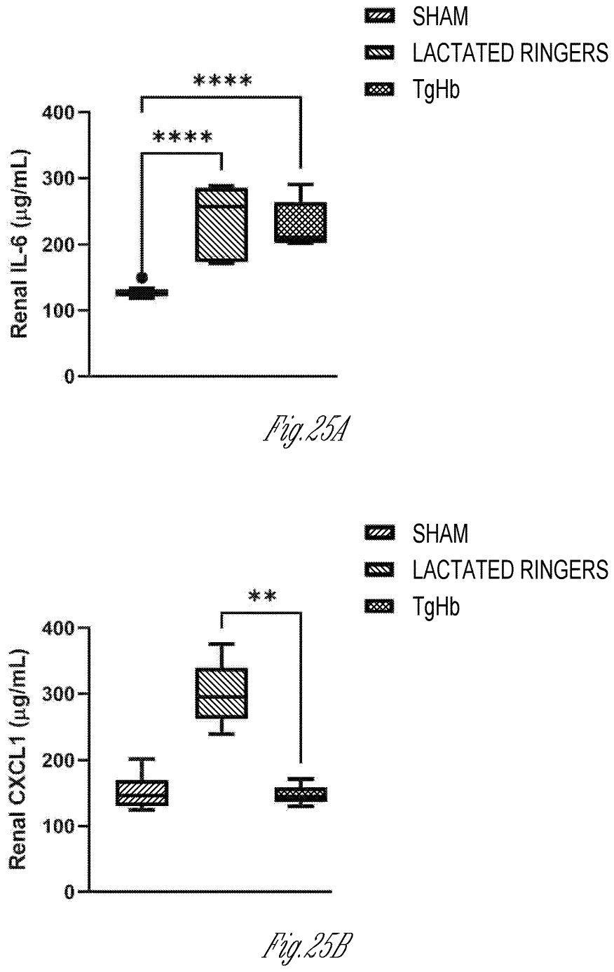

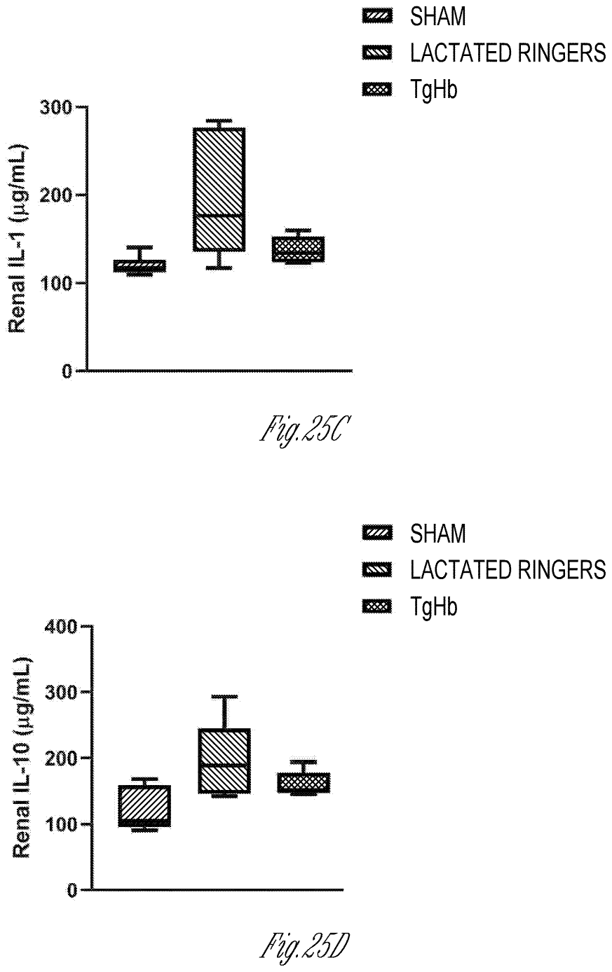

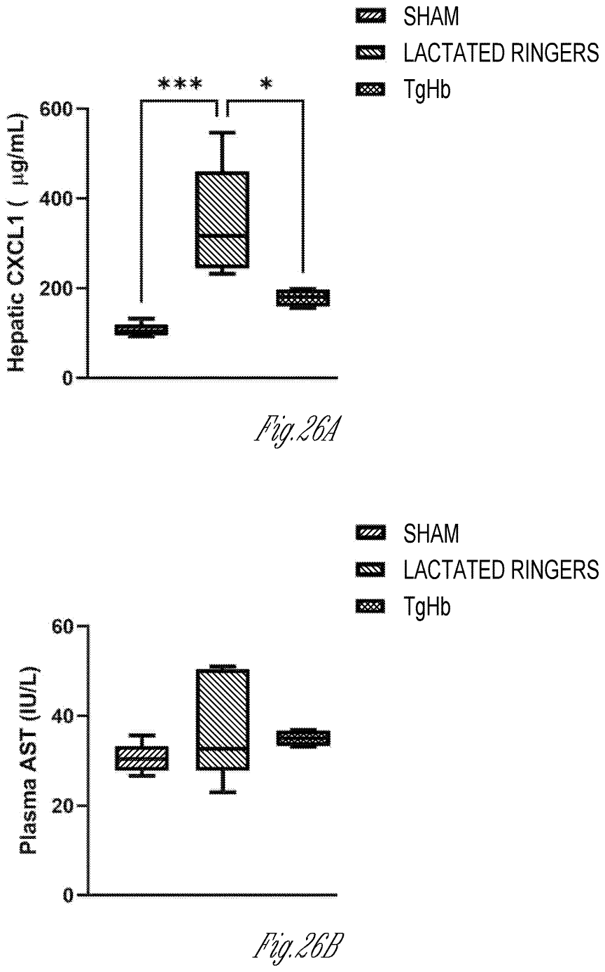

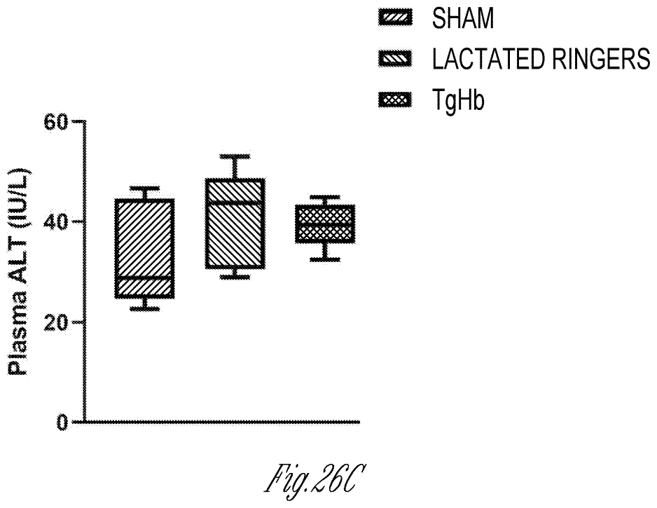

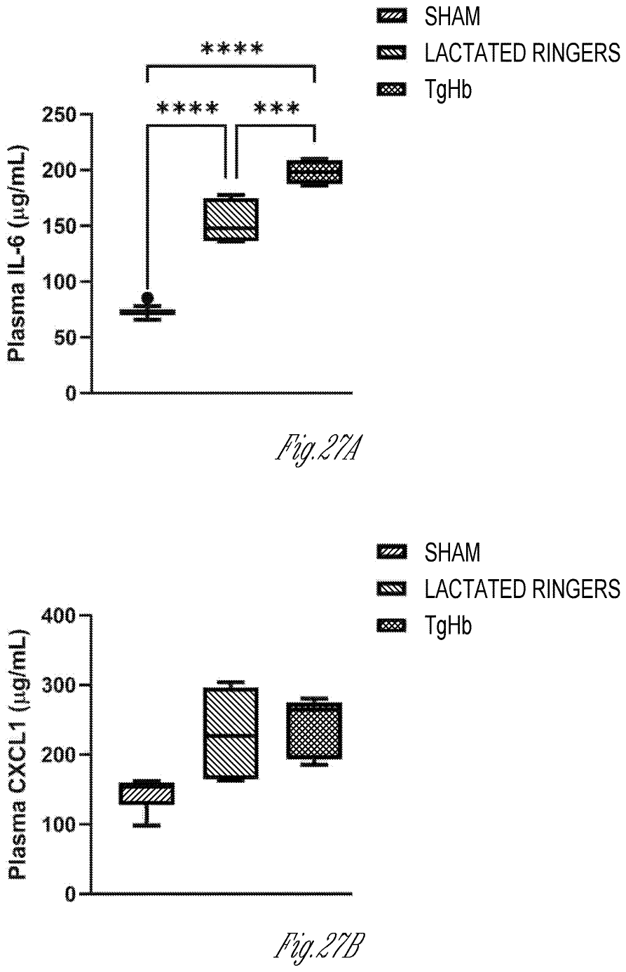

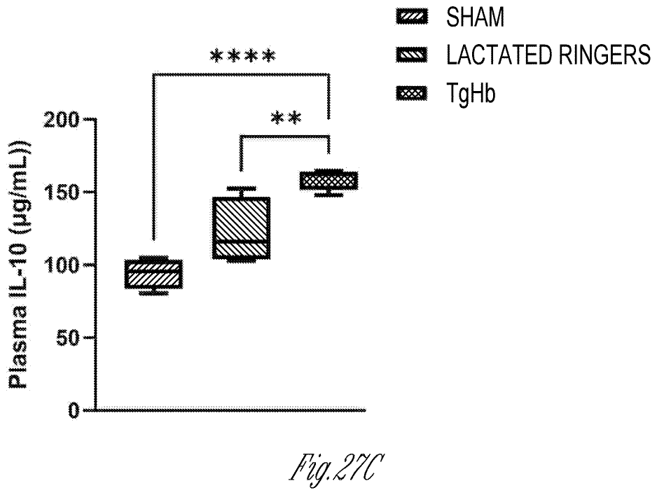

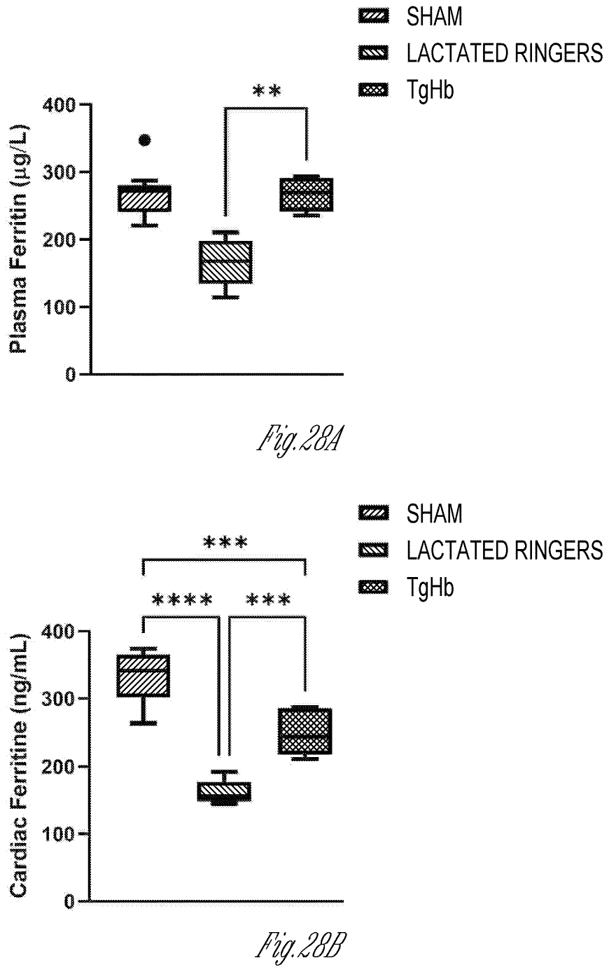

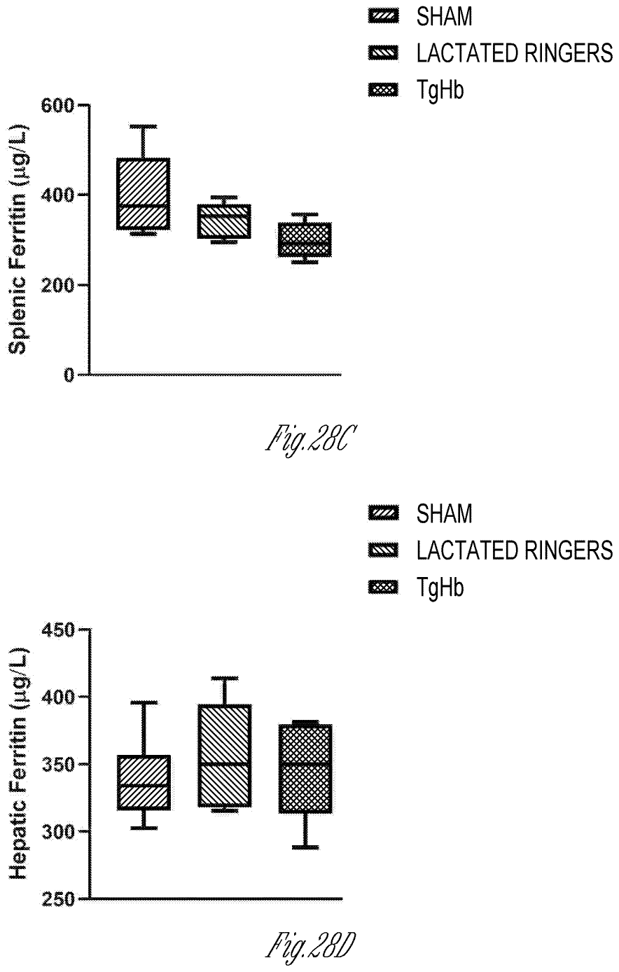

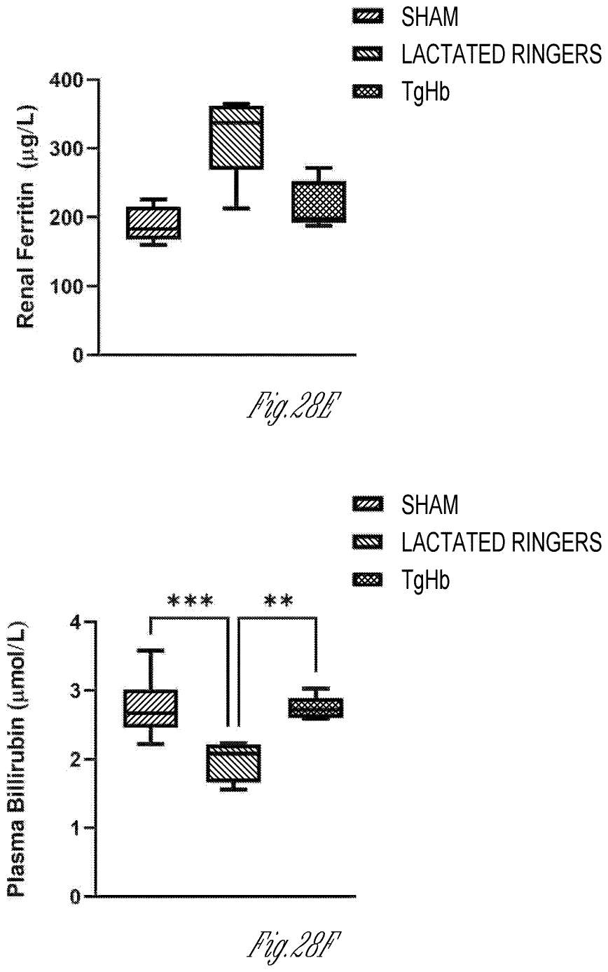

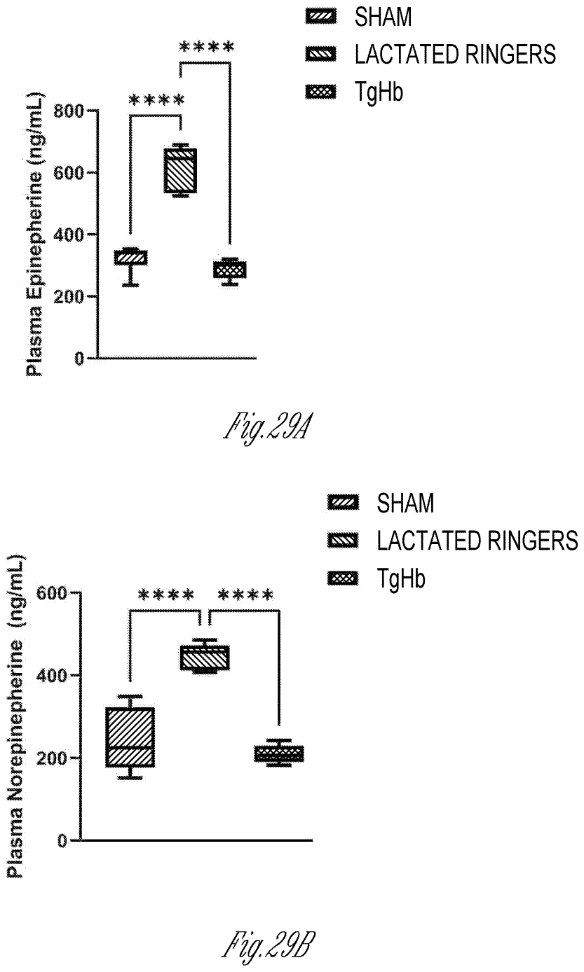

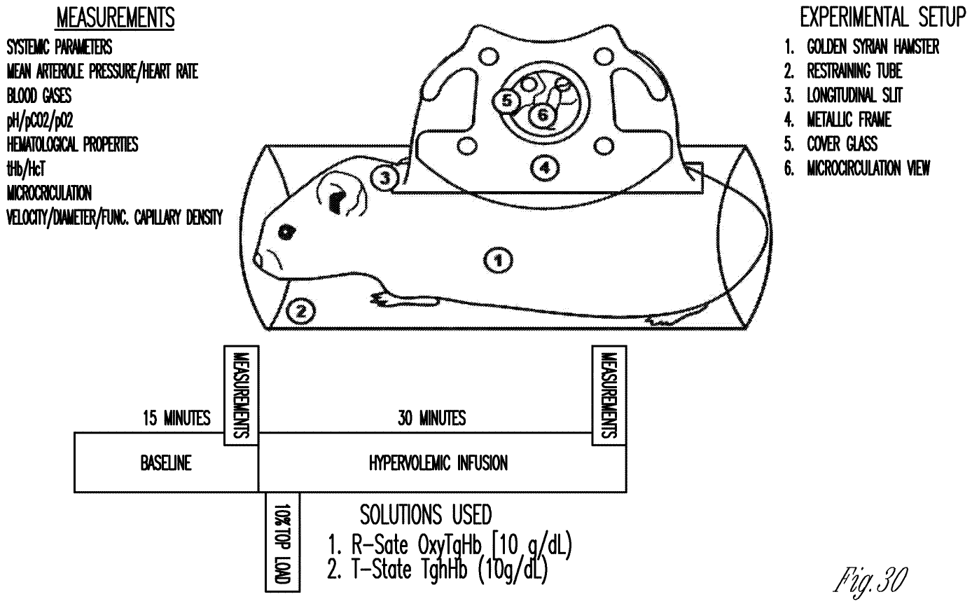

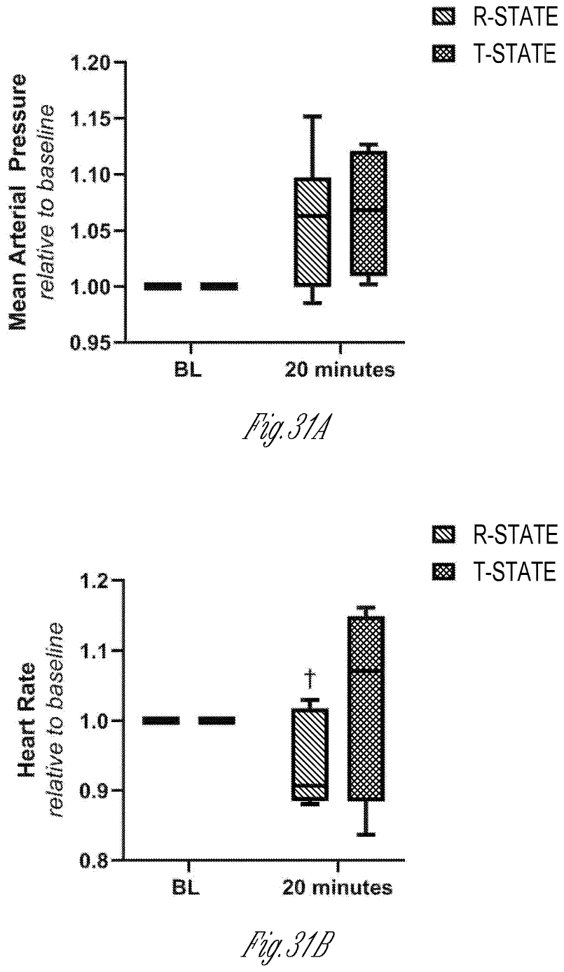

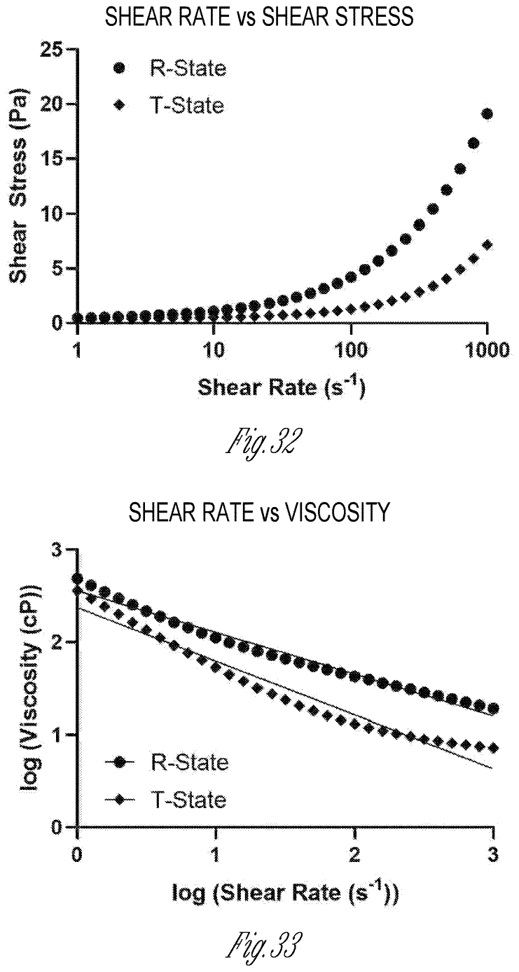

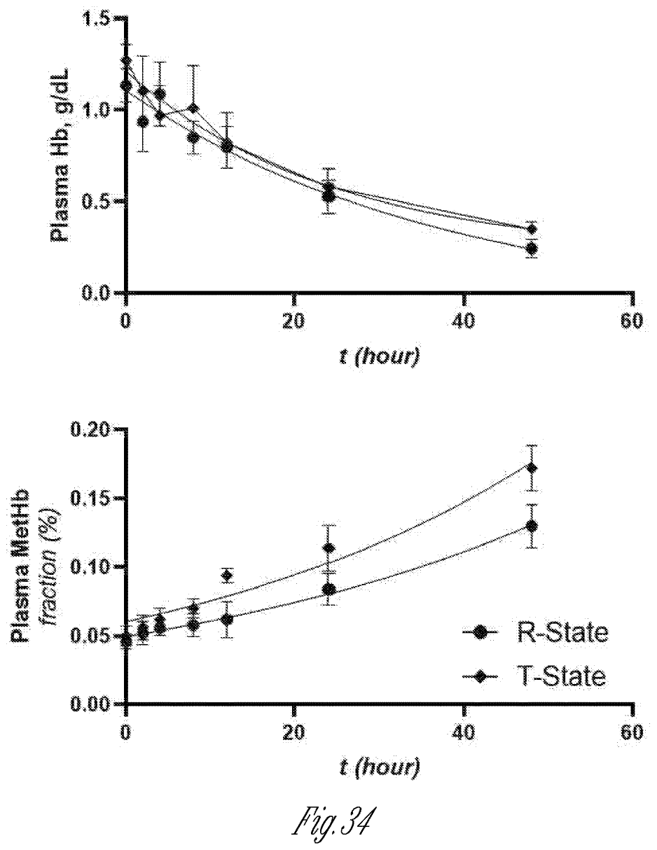

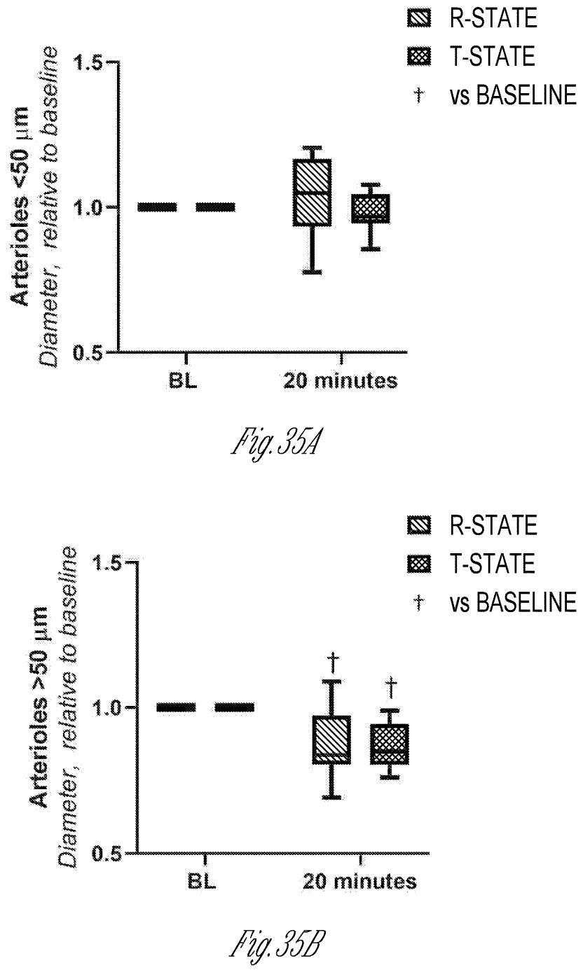

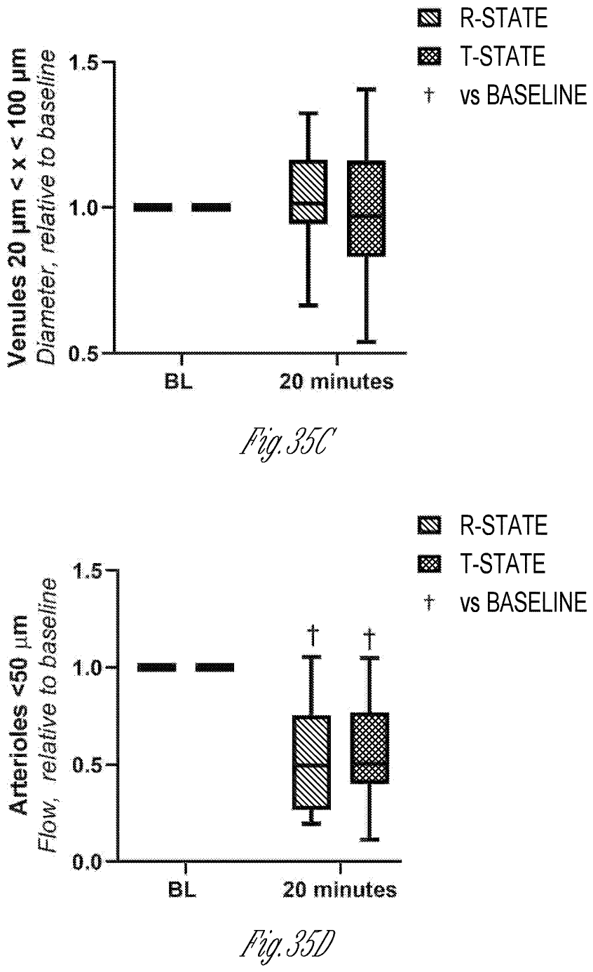

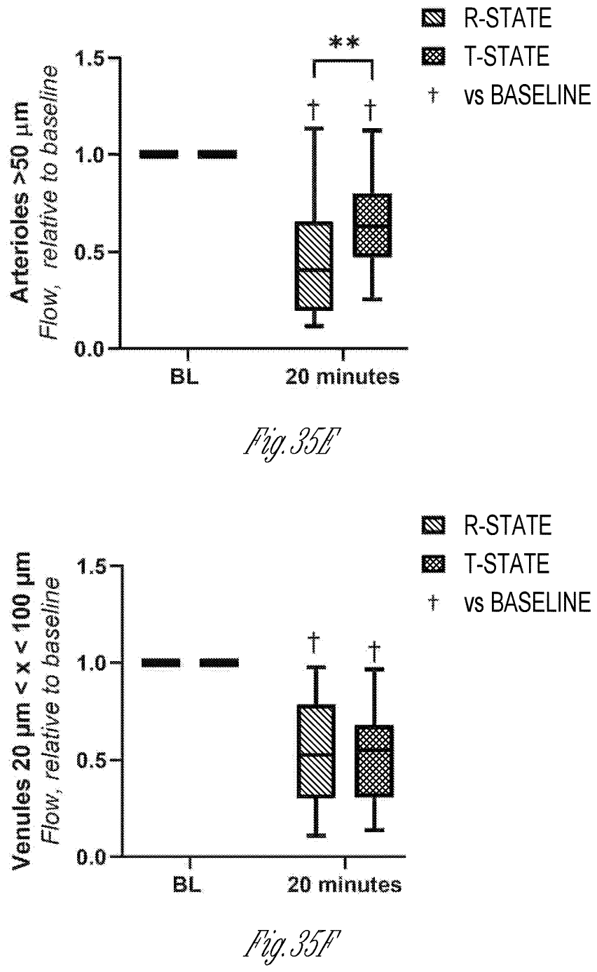

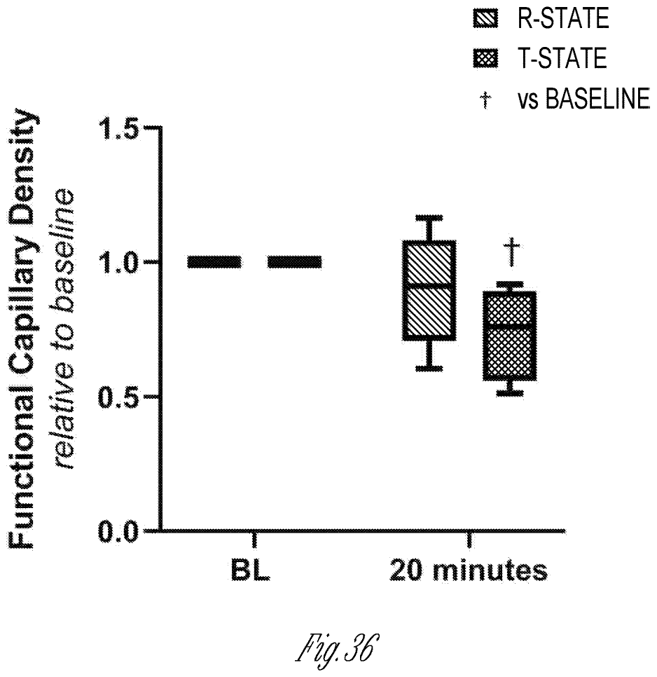

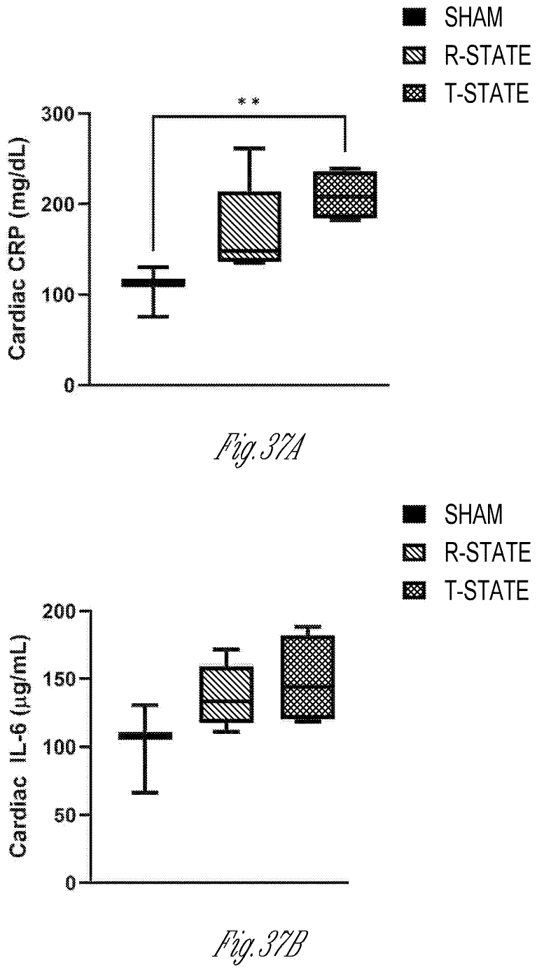

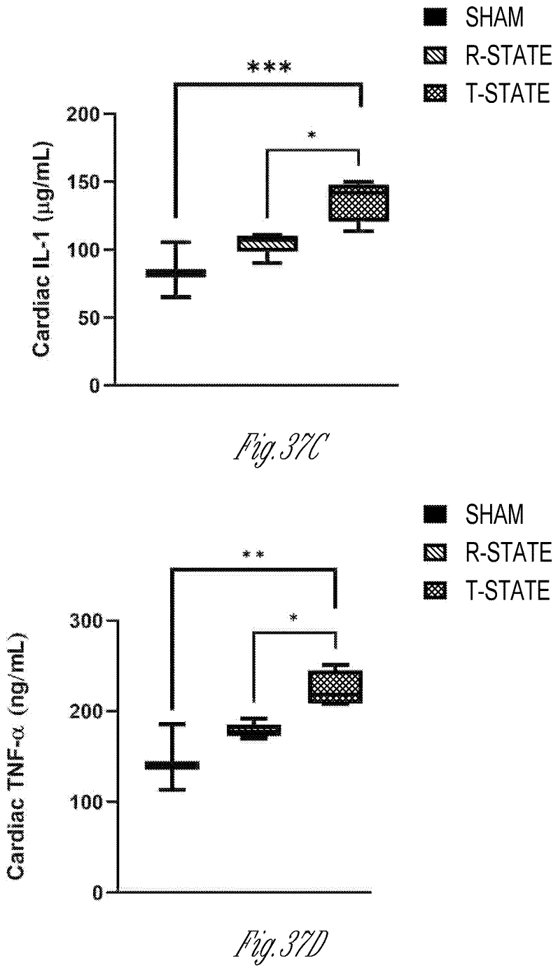

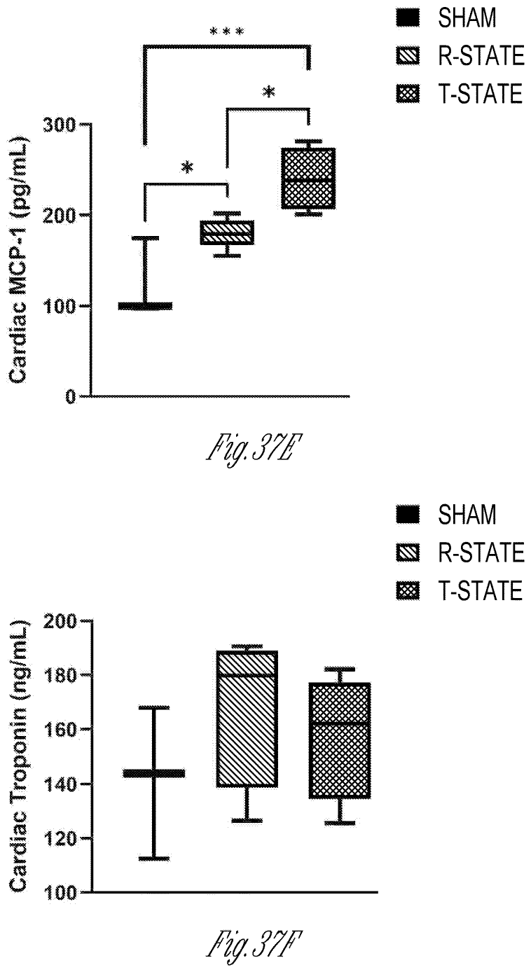

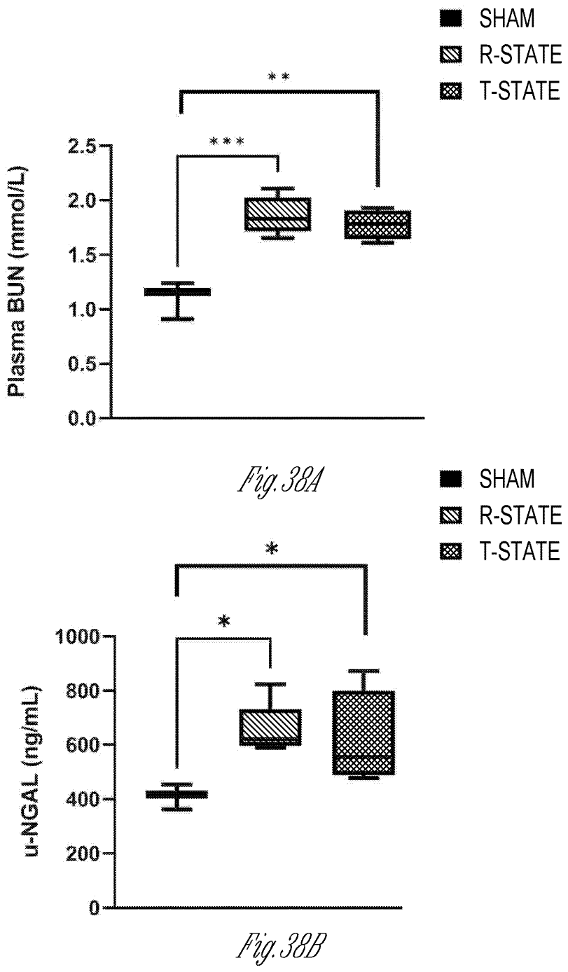

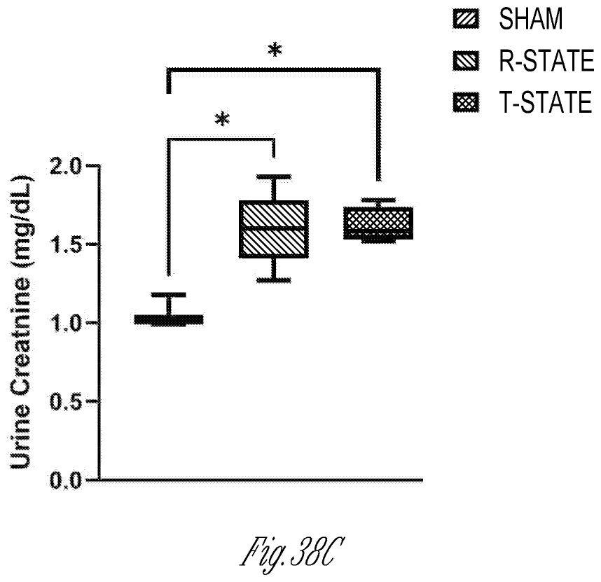

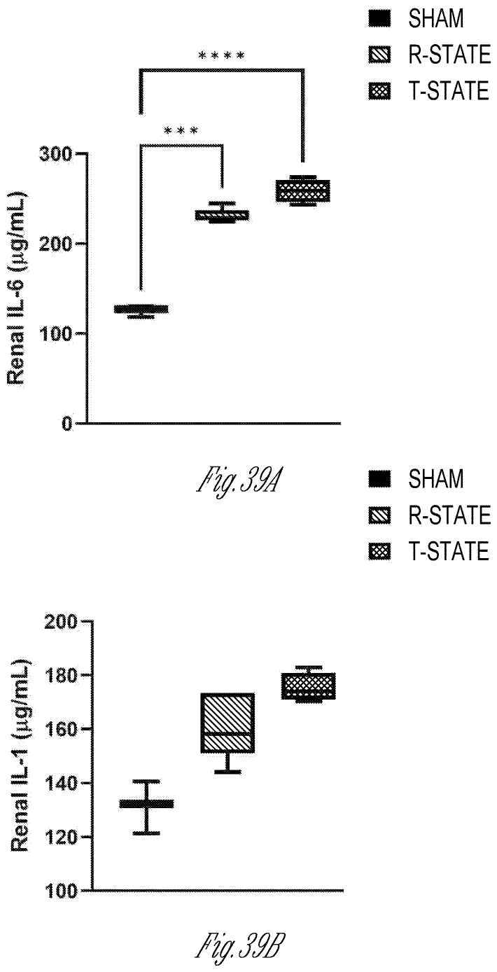

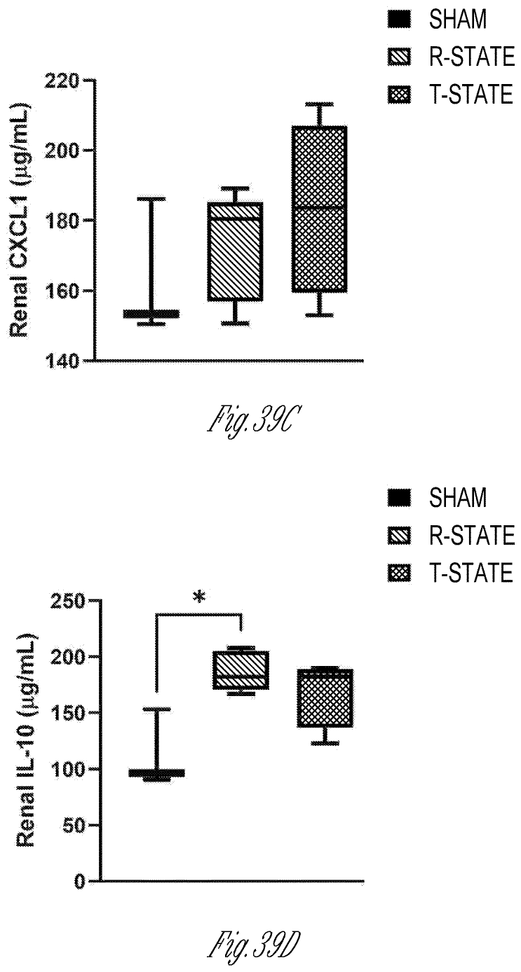

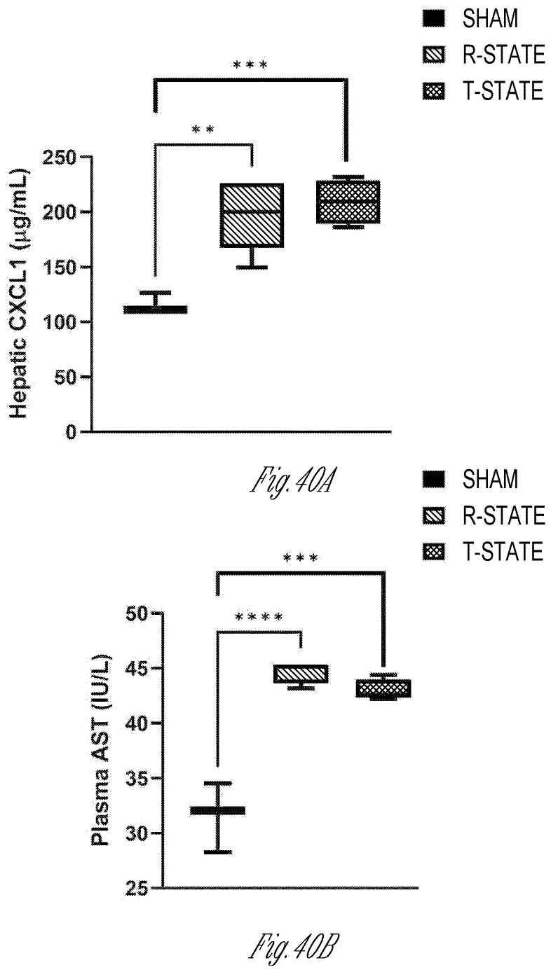

FIG. 3 illustrates the log (shear Rate (s⁻¹)) versus log (viscosity (cP)) and FIG. 4 illustrates shear rate (s⁻¹) versus shear stress (Pa) of both TgHb at 5 g/dL and TgHb at 10 g/dL.

Table 5 indicates the values of the transglutaminase to hemoglobin ratio, concentration of TgHb, metHb percentage, viscosity at 100 s⁻¹, and half-life in hours.

TABLE 5
Biophysical Properties of TgHb

| | TgHb |
|---|---|
| Transglutamase:Hb Ratio | 5:1 |
| Concentration (g/dL) | 10 |
| MetHb (%) | 5 |
| Viscosity (cP) | 5 |
| Half Life (Hrs) | 21 |

* Viscosity Measured at 100 s⁻¹

### Example 4: Pharmacokinetics and Oxidation Rate

Table 6 illustrates plasma Hb and plasma methHb fraction percentages over time from 0 hours to 48 hours.

TABLE 6
Pharmacokinetics of TgHb

| Time (hours) | Plasma Hb (g/dL) | Plasma metHb (%) |
|---|---|---|
| 0 | 1.21 ± 0.15 | 0.04 ± 0.01 |
| 2 | 1.11 ± 0.18 | 0.05 ± 0.01 |
| 4 | 1.19 ± 0.19 | 0.05 ± 0.01 |
| 8 | 1.01 ± 0.29 | 0.05 ± 0.01 |
| 12 | 0.84 ± 0.06 | 0.06 ± 0.01 |
| 24 | 0.63 ± 0.14 | 0.07 ± 0.01 |
| 48 | 0.38 ± 0.16 | 0.10 ± 0.01 |

FIG. 5 illustrates A) plasma Hb and B) plasma methHb fraction percentages over time from 0 hours to 48 hours.

### Example 3: Microvascular Hemodynamics

FIG. 6 indicates results of diameter and flow of the arterioles in the microcirculation relative to their baseline following the hypervolemic infusion protocol described above. The microhemodynamics include the diameter and flow of arterioles ranging from 20 μm-100 μm. The data was segregated by diameter into three groups <40 μm (small art), 40 μm-60 μm (medium art), and >60 μm (large art).

The diameter of the small arterioles (small art) after a hypervolemic infusion of HBOC-201 illustrated a significantly lower diameter than baseline and a significantly lower diameter compared to both hHIb or TgHb after their hypervolemic infusion. The flow in the small arterioles after a hypervolemic infusion of HBOC-201 illustrated a significantly lower flow than baseline and a significantly lower flow compared to TgHb after its hypervolemic infusion. The diameter of the medium arterioles (medium art) after a hypervolemic infusion of HBOC-201 illustrated a significantly lower diameter than TgHb after its hypervolemic infusion. The flow in the medium arterioles after a hypervolemic infusion of HBOC-201 illustrated a significantly lower flow than baseline, also it is illustrated that after the hypervolemic infusion of hHb or HBOC-201 the flow was significantly lower compared TgHb after its hypervolemic infusion. The diameter of the large arterioles (large art) after a hypervolemic infusion of hHb or HBOC-201 illustrated a significantly lower diameter than baseline. Also, following the hypervolemic infusion of HBOC-201 the diameter was significantly lower than hHb or TgHb after their hypervolemic infusion, furthermore, following the hypervolemic infusion of hHb the diameter was significantly lower than TgHb after its hypervolemic infusion. The flow in the large arterioles after a hypervolemic infusion of hHb or HBOC-201 illustrated a significantly lower flow than baseline and a significantly lower flow compared to TgHb after its hypervolemic infusion.

FIG. 7 indicates results of diameter and flow of the venules in the microcirculation relative to their baseline following the hypervolemic infusion protocol described above. The microhemodynamics include the diameter and flow of venules ranging from 20 μm-100 μm. The data was segregated by diameter into three groups <40 μm (small ven.), 40 μm-60 μm (medium ven.), and >60 μm (large ven.).

The venules did not show any significant differences to baseline or amongst each other in the diameter or flow in the small, medium, or large venules following the hypervolemic infusion of hHb, HBOC-201, or TgHb.

### Example 4: Functional Capillary Density

The number of functional capillaries was quantified 20 minutes after hypervolemic infusion protocol described above.

FIG. 8 indicates results of functional capillary density in Golden Syrian hamsters after the hypervolemic infusion of hHb, HBOC-201, or TgHb. Following the hypervolemic infusion of all solutions there was a significant drop in FCD compared to baseline. Following the hypervolemic infusion of hHb there was a significant drop in FCD when compared to TgHb after its hypervolemic infusion.

### Example 5: Heart Health

Following the conclusion of the hypervolemic infusion protocol the Golden Syrian Hamsters were sacrificed and the heart of each sacrificed hamster was analyzed. Cardiac c-reactive protein (CRP), cardiac interleukin-6 (IL-6), cardiac interleukin-10 (IL-10), cardiac tumor necrosis factor alpha (TNF-α), cardiac monocyte chemoattractant proteion-1 (MCP-1) and cardiac Troponin proteins were quantified in the tissue. More specifically, upon completion of the experiment A) cardiac CRP, B) cardiac IL-6, C) cardiac IL-10, D) cardiac TNF-a, E) cardiac MCP-1 and F) cardiac Troponin were measured to evaluate cardiac inflammation and function.

Table 7 indicates levels of cardiac CRP, cardiac IL-6, cardiac IL-10, cardiac TNF-α, cardiac MCP-1 and cardiac Troponin proteins that can indicate one or more status of the cardiac function. Also, three Golden Syrian Hamsters were sacrificed without undergoing any protocol to establish a control. Significant to sham (†), significant to HBOC-201 (‡), and significant to TgHb (&).

TABLE 7
20 Minutes After Hypovolemic Infusion

| | Sham | hHb | HBOC-201 | TgHb |
|---|---|---|---|---|
| Cardiac CRP (mg/dL) | 108.3 ± 27.9 | 270.4 ± 40.7^{†} | 236.1 ± 22.7^{†} | 216.3 ± 34.4^{†} |
| Cardiac IL-6 (μg/mL) | 101.5 ± 32.5 | 216.8 ± 55.1^{†} | 162.4 ± 22.1 | 183.0 ± 15.6 |
| Cardiac IL-10 (μg/mL) | 84.44 ± 20.3 | 179.5 ± 10.4^{†‡&} | 136.2 ± 17.2^{†} | 124.1 ± 19.5^{†} |
| Cardiac TNF-a (ng/mL) | 146.6 ± 36.4 | 291.6 ± 23.5^{†&} | 237.3 ± 24.3^{†} | 213.6 ± 9.8^{†} |
| Cardiac MCP-1 (pg/mL) | 124.1 ± 44.3 | 304.7 ± 49.9^{†} | 245.1 ± 15.2^{†} | 234.6 ± 40.6^{†} |
| Cardiac Troponin (ng/mL) | 141.5 ± 27.9 | 244.4 ± 22.3^{†&} | 184.1 ± 19.5 | 158.5 ± 11.2 |

^{†}vs sham,
^{‡}vs HBOC-201,
^{&}vs TgHb

FIG. 9 indicates levels of cardiac CRP, cardiac IL-6, cardiac IL-10, cardiac TNF-α, cardiac MCP-1 and cardiac Troponin proteins that can indicate one or more status of the cardiac function.

Cardiac CRP levels were significantly elevated compared to sham after the hypervolemic infusion of hHb, HBOC-201, or TgHb. Cardiac IL-6 levels were significantly elevated compared to sham after the hypervolemic infusion of hHb. Cardiac IL-10 levels were significantly elevated compared to sham after the hypervolemic infusion of hHb, HBOC-201, or TgHb. Cardiac IL-10 levels were significantly elevated after the hypervolemic infusion of hHb compared to the levels of IL-10 after the hypervolemic infusion of HBOC-201 and TgHb. Cardiac TNF-α levels were significantly elevated compared to sham after the hypervolemic infusion of hHb, HBOC-201, or TgHb. Cardiac TNF-α levels were significantly elevated after the hypervolemic infusion of hHb compared to the levels of TNF-α after the hypervolemic infusion of TgHb. Cardiac MCP-1 levels were significantly elevated compared to sham after the hypervolemic infusion of hHb, HBOC-201, or TgHb. Cardiac Troponin levels were significantly elevated compared to sham after the hypervolemic infusion of hHb. Cardiac Troponin levels were significantly elevated after the hypervolemic infusion of hHb compared to the cardiac Troponin levels from a hypervolemic infusion of TgHb.

### Example 6: Kidney Health

Upon completion of the experiment the Golden Syrian Hamsters were sacrificed where the kidney, urine and plasma were collected, A) plasma blood urea nitrogen BUN, B) urine neutrophil gelatinase-associated lipocalin NAGL, and C) urine creatinine was measured to evaluate kidney function. Kidney function can be speculated by quantifying the concentrations of plasma BUN, urine NGAL and urine creatinine. FIG. 10 indicates levels of concentrations of plasma BUN, urine NGAL and urine creatinine, which can be based to speculate one or more kidney functions. Also, three Golden Syrian Hamsters were sacrificed without undergoing any protocol to establish a control.

Table 8 indicates levels of plasma BUN, urine NGAL and urine creatinine proteins that can indicate one or more status of the kidney function. Also, three Golden Syrian Hamsters were sacrificed without undergoing any protocol to establish a control. Significant to sham (†), significant to HBOC-201 (‡), and significant to TgHb (&).

TABLE 8
20 Minutes After Hypovolemic Infusion

| | Sham | hHb | HBOC-201 | TgHb |
|---|---|---|---|---|
| Plasma BUN (mmol/L) | 1.1 ± 0.1 | 3.0 ± 0.3^{†‡&} | 2.4 ± 0.3^{†} | 2.0 ± 0.2^{†} |
| Urine NAGL (ng/mL) | 411.0 ± 46.1 | 871.9 ± 106.8^{†} | 695.0 ± 51.0^{†} | 709.4 ± 77.4^{†} |
| Urine Creatinine (mg/dL) | 1.1 ± 0.1 | 1.9 ± 0.2^{†} | 1.7 ± 0.3 | 1.6 ± 0.2 |

^{†}vs sham,
^{‡}vs HBOC-201,
^{&}vs TgHb

Plasma BUN levels were significantly higher compared to sham after a hypovolemic infusion of hHb, HBOC-201, or TgHb. Furthermore, following the hypervolemic infusion of hHb levels of plasma BUN were significantly higher than the values recorded after a hypervolemic infusion of HBOC-201 or TgHb. Urine NAGL levels were significantly higher compared to sham after a hypervolemic infusion of hHb, HBOC-201, or TgHb. Urine creatinine levels were significantly higher compared to sham after a hypovolemic infusion of hHb.

Kidney inflammatory markers were quantified in the form of a variety of interleukin (IL) and chemokine (C—X—C motif) ligand 1 (CXCL1) markers renal IL-6, renal IL-1, renal CXCL1 and renal IL-10. More specifically, upon completion of the experiment A) renal IL-6, B) renal IL-1, C) renal CXCL1, and D) renal IL-10 were measured to evaluate kidney inflammation.

Upon completion of the experiment the Golden Syrian Hamsters were sacrificed where the kidney, was collected, A) renal IL-6, B) renal IL-1, C) renal CXCL1 and D) renal IL-10 was measured to evaluate kidney inflammation. Kidney inflammation can be speculated by quantifying the concentrations of renal IL-6, renal IL-1, renal CXCL1, and renal IL-10. FIG. 11 indicates levels of concentrations of renal IL-6, renal IL-1, renal CXCL1, and renal IL-10, which can be based to speculate one or more kidney inflammation. Also, three Golden Syrian Hamsters were sacrificed without undergoing any protocol to establish a control.

Table 9 indicates levels of renal IL-6, renal IL-1, renal CXCL1 and renal IL-10 proteins that can indicate one or more status of the kidney inflammation. Also, three Golden Syrian Hamsters were sacrificed without undergoing any protocol to establish a control. Significant to sham (†), significant to HBOC-201 (‡), and significant to TgHb (&).

TABLE 9
20 Minutes After Hypovolemic Infusion

| | Sham | hHb | HBOC-201 | TgHb |
|---|---|---|---|---|
| Renal IL-6 (μg/mL) | 125.5 ± 6.1 | 388.2 ± 59.7^{†‡&} | 287.7 ± 32.5^{†} | 277.7 ± 39.9^{†} |
| Renal IL-1 (μg/mL) | 131.4 ± 9.7 | 241.0 ± 47.4^{†&} | 183.4 ± 18.8 | 180.7 ± 26.4 |
| Renal CLCX1 (μg/mL) | 163.4 ± 19.8 | 319.6 ± 64.6^{†‡&} | 231.9 ± 31.3 | 180.4 ± 22.9 |
| Renal IL-10 (μg/mL) | 113.5 ± 34.6 | 266.5 ± 9.4^{†‡&} | 197.1 ± 12.7^{†} | 158.9 ± 21.0 |

^{†}vs sham,
^{‡}vs HBOC-201,
^{&}vs TgHb

Renal IL-6 levels were significantly higher compared to the sham following the hypervolemic infusion of hHb, HBOC-201, or TgHb. Furthermore, following a hypervolemic infusion of hHb renal IL-6 levels were significantly higher compared to the levels following a hypervolemic infusion of HBOC-201 or TgHb. Renal IL-1 levels were significantly higher compared to sham following a hypervolemic infusion of hHb. Furthermore, renal IL-1 levels were significantly higher following a hypervolemic infusion of hHb compared to renal IL-1 levels following a hypervolemic infusion of TgHb. Renal CXCL1 levels were significantly higher compared to sham following the hypervolemic infusion of hHb. Renal CXCL1 levels were significantly higher following a hypervolemic infusion of hHb compared to CXCL1 levels following a hypervolemic infusion of HBOC-201 or TgHb. Renal IL-10 levels were significantly higher than sham following a hypervolemic infusion of hHb or HBOC-201. Renal IL-10 levels were significantly higher after a hypervolemic infusion of hHb compared to the renal IL-10 levels following a hypervolemic infusion of HBOC-201 or TgHb.

### Example 7: Liver Health

Upon completion of the experiment A) Hepatic CXCL1, B) plasma aspartate transaminase (AST), and C) plasma alanine transaminase (ALT) was measured to evaluate liver function and inflammation of the liver. FIG. 12 indicates hepatic CXCL1, plasma AST and plasma ALT concentrations, which can be based to speculate to be associated with liver inflammation and function.

Table 10 indicates levels of hepatic CXCL1, plasma AST and plasma ALT proteins that can indicate one or more status of the liver inflammation and liver function. Also, three Golden Syrian Hamsters were sacrificed without undergoing any protocol to establish a control. Significant to sham (†), significant to HBOC-201 (‡), and significant to TgHb (&).

TABLE 10
20 Minutes After Hypovolemic Infusion

| | Sham | hHb | HBOC-201 | TgHb |
|---|---|---|---|---|
| Hepatic CXCL1 (μg/mL) | 116.1 ± 9.1 | 298.9 ± 55.7^{†&} | 244.2 ± 17.0^{†} | 184.8 ± 13.6 |
| Plasma AST (IU/L) | 31.6 ± 3.2 | 53.2 ± 1.3^{†&} | 46.1 ± 3.4^{†&} | 37.3 ± 3.1 |
| plasma ALT (IU/L) | 45.3 ± 2.1 | 46.2 ± 1.7 | 41.5 ± 1.5 | 41.5 ± 2.6 |

^{†}vs sham,
‡ vs HBOC-201,
^{&}vs TgHb

Hepatic CXCL1 levels were significantly higher compared to the sham following a hypervolemic infusion of hHb or HBOC-201. Furthermore, hepatic CXCL1 levels following a hypervolemic infusion of hHb was significantly higher compared to the hepatic CXCL1 levels following a hypervolemic infusion of TgHb. Plasma AST levels were higher compared to sham following a hypervolemic infusion of hHb or HBOC-201. Furthermore, plasma AST levels following a hypervolemic infusion of hHb or HBOC-201 were significantly higher compared to plasma AST levels following a hypervolemic infusion of TgHb. Plasma ALT levels has no significant changes to sham or between groups following a hypervolemic infusion of hHb, HBOC-201, or TgHb.

### Example 8: Vasculature Health

Following the conclusion of the experiment plasma was collected, plasma IL-6, plasma CXCL1, and plasma IL-10 were quantified. FIG. 13 indicates A) plasma IL-6, B) plasma CXCL1, and C) plasma IL-10 which can be speculated to be associated with vascular health.

Table 11 indicates levels of plasma IL-6, plasma CXCL1, and plasma IL-10 proteins that can indicate one or more status of the liver inflammation and liver function. Also, three Golden Syrian Hamsters were sacrificed without undergoing any protocol to establish a control. Significant to sham (†), significant to HBOC-201 (‡), and significant to TgHb (&).

TABLE 11
20 Minutes After Hypovolemic Infusion

| | Sham | hHb | HBOC-201 | TgHb |
|---|---|---|---|---|
| Plasma IL-6 (μg/mL) | 69.9 ± 3.6 | 341.5 ± 20.0^{†&} | 331.1 ± 26.6^{†&} | 254.8 ± 19.4^{†} |
| Plasma CLCX1 (μg/mL) | 146.9 ± 22.9 | 351.2 ± 74.6^{†} | 308.2 ± 26.2^{†} | 256.6 ± 24.3 |
| Plasma IL-10 (μg/mL) | 96.2 ± 13.6 | 282.5 ± 27.8^{†‡&} | 229.4 ± 9.9^{†} | 204.1 ± 10.7^{†} |

^{†}vs sham,
^{‡}vs HBOC-201,
^{&}vs TgHb

Plasma IL-6 levels were significantly higher compared to the sham following a hypervolemic infusion of hHb, HBOC-201, or TgHb. Furthermore, following the hypervolemic infusion of hHb or HBOC-201 plasma IL-6 levels were significantly higher compared to the plasma IL-6 levels following the hypervolemic infusion of TgHb. Plasma CXCL1 levels were significantly higher compared to sham following a hypervolemic infusion of hHb or HBOC-201. Plasma IL-10 levels were significantly higher compared to sham following a hypervolemic infusion of hHb, HBOC-201, and TgHb. Furthermore, the plasma IL-10 levels following a hypervolemic infusion of hHlb was significantly higher compared to the plasma IL-10 levels following a hypervolemic infusion of HBOC-201 or TgHb.

### Example 9: Ferritin and Bilirubin Concentration

Following the conclusion of the experiment the ferritin and bilirubin concentration was quantified in the plasma, heart, spleen, liver, kidney and the ferritin concentration was quantified. FIG. 14 indicates A) plasma ferritin, B) cardiac ferritin, C) splenic ferritin D) hepatic ferritin, E) renal ferritin, F) plasma bilirubin which can be speculated to be associated with levels of iron in the circulation.

Table 12 indicates levels of plasma ferritin, cardiac ferritin, splenic ferritin, hepatic ferritin, renal ferritin, and plasma bilirubin proteins that can indicate iron concentration and levels of hemolysis in the vasculature and vital organs. Also, three Golden Syrian Hamsters were sacrificed without undergoing any protocol to establish a control. Significant to sham (†), significant to HBOC-201 (‡), and significant to TgHb (&).

TABLE 12
20 Minutes After Hypovolemic Infusion

| | Sham | hHb | HBOC-201 | TgHb |
|---|---|---|---|---|
| Plasma Ferritin (μg/L) | 233.9 ± 21.3 | 358.1 ± 48.3^{†&} | 326.4 ± 19.4^{&} | 232.0 ± 12.1 |
| Cardiac Ferritin (ng/L) | 311.1 ± 17.7 | 337.1 ± 62.3 | 285.0 ± 29.8 | 324.3 ± 15.2 |
| Splenic Ferritin (μg/L) | 358.7 ± 38.3 | 402.0 ± 15.37 | 351.2 ± 42.8 | 415.8 ± 58.7 |
| Hepatic Ferritin (μg/L) | 326.8 ± 23.9 | 619.5 ± 116.8^{†&} | 465.5 ± 27.7 | 435.8 ± 91.6 |
| Renal Ferritin (μg/L) | 194.7 ± 27.3 | 258.9 ± 44.9 | 295.4 ± 31.1 | 250.9 ± 22.4 |
| Plasma Bilirubin (μmol/L) | 2.87 ± 0.26 | 4.85 ± 0.65^{†&} | 3.55 ± 0.45 | 2.94 ± 0.47 |

^{†}vs sham,
‡ vs HBOC-201,
^{&}vs TgHb

Plasma ferritin levels were significantly higher compared to sham following a hypervolemic infusion of hHb. Furthermore, plasma ferritin levels were significantly higher following a hypervolemic infusion of hHb compared to the plasma ferritin levels following a hypervolemic infusion of TgHb. Lastly, plasma ferritin levels were significantly higher following a hypervolemic infusion of HBOC-201 compared to the plasma ferritin levels following a hypervolemic infusion of TgHb. There was no significant difference in cardiac ferritin levels compared to sham or between groups following a hypervolemic infusion of hHb, HBOC-201, or TgHb. There were no significant differences in splenic ferritin levels compared to sham or between groups following a hypervolemic infusion of hHb, HBOC-201, or TgHb. Hepatic ferritin levels were significantly higher compared to sham following a hypervolemic infusion of hHb. Furthermore, hepatic ferritin levels were significantly higher compared following a hypervolemic infusion of hHb compared to the hepatic ferritin levels following a hypervolemic infusion of TgHb. There were no significant differences in renal ferritin compared to sham or between groups following a hypervolemic infusion of hHb, HBOC-201, or TgHb. Plasma bilirubin levels were significantly higher compared to sham following a hypervolemic infusion of hHb. Furthermore, plasma bilirubin levels were significantly higher following a hypervolemic infusion of hHb compared to the plasma bilirubin levels following a hypervolemic infusion of TgHb.

### Example 10: Catecholamines

Following the conclusion of the experiment the plasma epinephrine and plasma norepinephrine concentration was quantified in the plasma. FIG. 15 indicates A) plasma epinephrine and B) plasma norepinephrine which can be speculated to be associated with levels of the nervous system.

Table 13 indicates levels of plasma epinephrine and plasma norepinephrine proteins that can indicate iron concentration and levels of nervous system reactions. Also, three Golden Syrian Hamsters were sacrificed without undergoing any protocol to establish a control. Significant to sham significant to HBOC-201 (‡), and significant to TgHb (&).

TABLE 13
20 Minutes After Injection

| | Sham | hHb | HBOC-201 | TgHb |
|---|---|---|---|---|
| Plasma Epinephrine (ng/mL) | 330.8 ± 17.4 | 423.8 ± 62.9 | 350.4 ± 23.7 | 354.5 ± 73.7 |
| Plasma Norepinephrine (ng/mL) | 330.8 ± 25.4 | 322.4 ± 30.0 | 308.8 ± 15.4 | 233.5 ± 32.1 |

† vs sham,
‡ vs HBOC-201,
& vs TgHb

There was no significant difference in plasma epinephrine levels compared to sham or between groups following a hypervolemic infusion of hHb, HBOC-201, or TgHb. There was no significant difference in plasma norepinephrine levels compared to sham or between groups following a hypervolemic infusion of hHb, HBOC-201, or TgHb.

### Example New 2—Controlled Hemorrhagic Shock Resuscitation with TgHlb vs. Lactated Ringers

For the following EXAMPLES 11-19, the efficacy of this hemoglobin-based oxygen carrier using transglutaminase polymerization (TgHb) as a resuscitative fluid was tested en-vivo using the Golden Syrian hamsters.

### Materials and Methods

### Hb Preparation

Hb (P68871, P69905) for this study was prepared via tangential flow filtration as previously described. (Palmer, A. F.; Sun, G.; Harris, D. R. Tangential Flow Filtration of Hemoglobin. Biotechnol. Prog. 2009, 25(1), 189-199. https://doi.org/10.1002/btpr.119). The concentration of Hb was determined spectrophotometrically.

### Polymerization of Hb Using Transglutaminase

Hemoglobin protein dispersions were prepared at either 20% solids content (w/v) and adjusted to pH 7.5. The protein substrate was mixed with microbial transglutaminase at a ratio of approximately 5 units per gram of hemoglobin and slowly agitated in a rotating incubator at 37° C. At timed intervals, samples were taken for SDS-PAGE and OPA analyses. In most cases, the soluble supernatants obtained after centrifugation at 11950 g were tested. Residual protein content in transglutaminase-treated samples that were kept at 37° C. for 24 hours was measured using the BCA method.

### Window Chamber Preparation in Golden Syrian Hamsters

Investigations were performed in 55-65 g male Golden Syrian Hamsters (Charles River Laboratories, Boston, MA) fitted with a dorsal skinfold window chamber. Animal handling and care followed the NIH Guide for the Care and Use of Laboratory Animals. The experimental protocol was approved by the local animal care committee. The hamster window chamber model is widely used for microvascular studies in the unanesthetized state, and the complete surgical technique is described in detail elsewhere. (Colantuoni, A.; Bertuglia, S.; Intaglietta, M. Quantitation of Rhythmic Diameter Changes in Arterial Microcirculation. Am. J. Physiol. 1984, 246 (4 Pt 2), H508-517. https://doi.org/10.1152/ajpheart. 1984.246.4.H508.) The experimental animal was allowed at least 2 days for recovery before the preparation was assessed under the microscope for any signs of edema, bleeding, or unusual neovascularization. Animals were anesthetized again, and arterial and venous catheters filled with a heparinized saline solution (30 IU/ml) were implanted. Catheters were tunneled under the skin, exteriorized at the dorsal side of the neck, and securely attached to the window frame. The microvasculature was examined 3 to 4 days after the initial surgery and only animals with window chambers whose tissue did not present regions of low perfusion, inflammation, and edema were entered into the study. (Tsai, A. G.; Friesenecker, B.; McCarthy, M.; Sakai, H.; Intaglietta, M. Plasma Viscosity Regulates Capillary Perfusion during Extreme Hemodilution in Hamster Skinfold Model. Am. J. Physiol. 1998, 275 (6), H2170-2180)

### Inclusion Criteria

Animals were considered suitable for experiments if systemic parameters were as follows: heart rate (HR)>340 beats/min, mean arterial blood pressure (MAP)>80 mm Hg, systemic Hct>45%, and arterial O2 partial pressure (pAO2)>50 mm Hg. Additionally, animals with signs of low perfusion, inflammation, edema, or bleeding in their microvasculature were excluded from the study.

### Experimental Setup

The unanesthetized animal was placed in a restraining tube with a longitudinal slit from which the window chamber protruded, then fixed to the microscopic stage for transillumination with the intravital microscope (BX51WI, Olympus, Japan). Animals were given 20 minutes to adjust to the tube environment and images were obtained using a CCD camera (4815, COHU, San Diego, CA). Measurements were carried out using a 40×(LUMPFL-WIR, numerical aperture 0.8, Olympus) water immersion objective.

### Systemic Parameters

The MAP and HR were monitored continuously (MP150, Biopac System Inc., Santa Barbara, CA). Hct was measured from centrifuged arterial blood samples taken in heparinized capillary tubes. Hb content was determined spectrophotometrically (B-Hemoglobin, Hemocue, Stockholm, Sweden). Arterial blood was collected in heparinized glass capillaries (50 μL) and immediately analyzed for pO2, pCO2, and pH (ABL90; Radiometer America, Brea, CA). Arterial Hb saturation was measured using an IL482 CO-Oximeter (Instrumentation Laboratory, Lexington, MA).

### Microhemodynamics

Arteriolar and venular blood flow velocities were measured online by using the photodiode cross-correlation method (Intaglietta, M.; Silverman, N. R.; Tompkins, W. R. Capillary Flow Velocity Measurements in Vivo and in Situ by Television Methods. Microvasc. Res. 1975, 10 (2), 165-179. https://doi.org/10.1016/0026-2862 (75) 90004-7) (Photo Diode/Velocity Tracker Model 102B, Vista Electronics, San Diego, CA). The measured centerline velocity (V) was corrected according to vessel size to obtain the mean RBC velocity. (Lipowsky, H. H.; Zweifach, B. W. Application of the “Two-Slit” Photometric Technique to the Measurement of Microvascular Volumetric Flow Rates. Microvasc. Res. 1978, 15 (1), 93-101. https://doi.org/10.1016/0026-2862 (78) 90009-2.) A video image-shearing method was used to measure vessel diameter (D). (Intaglietta, M.; Tompkins, W. R. Microvascular Measurements by Video Image Shearing and Splitting. Microvasc. Res. 1973, 5 (3), 309-312. (2) Lipowsky, H. H.; Zweifach, B. W. Application of the “Two-Slit” Photometric Technique to the Measurement of Microvascular Volumetric Flow Rates. Microvasc. Res. 1978, 15 (1), 93-101. https://doi.org/10.1016/0026-2862 (78) 90009-2.) Blood flow (Q) was calculated from the values measured as Q=π×V(D/2)\hat{ }2. Changes in arteriolar and venular diameter from baseline were used as indicators of a change in vascular tone. This calculation assumes a parabolic velocity profile and has been found to be applicable to tubes of 15-100 μm internal diameters and for Hets in the range of 6-60%. (Lipowsky, H. H.; Zweifach, B. W. Application of the “Two-Slit” Photometric Technique to the Measurement of Microvascular Volumetric Flow Rates. Microvasc. Res. 1978, 15 (1), 93-101. https://doi.org/10.1016/0026-2862 (78) 90009-2)

### Functional Capillary Density (FCD)

Functional capillaries, defined as capillary segments that have transit of at least one RBC in a 60 second period in 10 successive microscopic fields, were assessed in a region of 0.46 mm². The FCD (cm-1) was calculated as the total length of RBC perfused capillaries divided by the viewing area (0.46 mm²).

### Acute Hemorrhage and Volume Replacement Protocol

Acute hemorrhage was induced by withdrawing 50% of the estimated total blood volume (BV) via the carotid artery catheter within 5 min. The timeline of protocol can be found in FIG. 16. Total blood volume (BV) was estimated as 7% of body weight. Thirty minutes after hemorrhage induction, animals were randomized and received a single volume infusion of 50% of the shed blood volume (25% of blood volume) of the resuscitation fluid (see experimental groups) over 7 min via the jugular vein catheter. Restoration of 25% of the blood volume does not cause hypervolemia (reinstating normovolemia) in the hamster window model, because autotransfusion (from the extravascular space) restores about half of the shed volume during the shock period. Animals did not receive any additional fluid during the experiment. Variables were recorded before hemorrhage (baseline), after hemorrhage (shock), 30 minutes after volume replacement (resuscitation) and 60 min after volume replacement (resuscitation).

### Experimental Groups

Animals were divided randomly into two experimental groups following the 30 minutes in hemorrhagic shock: 1) TghHb and 2) Lactated Ringer's.

### Oxygen Saturation in the Microcirculation

The animal's dorsal window chamber was imaged using a Pika L hyperspectral imaging system (Resonon Inc, Bozeman, MT) from which hyperspectral images (HSI) of the microcirculation were acquired. The methods for analyzing these images have been previously described.( ) Lucas, A.; Ao-ieong, E. S. Y.; Williams, A. T.; Jani, V. P.; Muller, C. R.; Yalcin, O.; Cabrales, P. Increased Hemoglobin Oxygen Affinity With 5-Hydroxymethylfurfural Supports Cardiac Function During Severe Hypoxia. Front. Physiol. 2019, 10.) Images were taken at baseline, shock, 30 min post resuscitation, and 60 min post resuscitation.

### Organ Harvesting and Analysis for Markers of Organ Damage and Inflammation

Blood was collected and centrifuged to separate the plasma. Animals were sacrificed with Fatal Plus (sodium pentobarbital, 300 mg/kg), urine was collected, and heart, kidneys, liver, and spleen were harvested. Markers of inflammation, function, and organ injury were evaluated using the ELISA Kits/Assays described in Table 1.

### Statistical Analysis

Results are presented as mean±standard deviation. Data within each group was analyzed using analysis of variance for repeated measurements (ANOVA, Kruskal-Wallis test). Microhemodynamic measurements were compared to baseline levels obtained before the experimental procedure. The box-whisker plot separates the data into quartiles, with the top of the box defining the 75th percentile, the line within the box giving the median, and the bottom of the box showing the 25th percentile. The upper “whisker” defines the 95^{th} percentile and the lower whisker defines the 5th percentile. Microhemodynamic data is presented as absolute values and ratios relative to baseline values. A ratio of 1.0 signifies no change from baseline while lower and higher ratios are indicative of changes proportionally lower and higher than baseline (i.e., 1.5 would mean a 50% increase from the baseline level). The same vessels and capillary fields were followed so that direct comparisons to their baseline levels could be performed, allowing for more robust statistics for small sample populations. All statistics were calculated using GraphPad Prism 4.01 (GraphPad Software, Inc., San Diego, CA). Changes were considered statistically significant if p<0.05.

### Results

### Mean Arterial Pressure (MAP), Heart Rate (HR), Hematological Properties and Blood Gases

Table 14 indicates hematocrit (Hct), total hemoglobin concentration ((Hb), mean arterial pressure (MAP), heart rate (HR), arterial partial pressure of oxygen (pO2), partial pressure of carbon dioxide (pCO2), arterial pH, concentration of lactate (cLac) at baseline, shock, 30 minutes after resuscitation, and 60 minutes after resuscitation.

TABLE 14
Acute Hemorrhage and Volume Replacement

| | | | 30-Minutes | | 60-Minutes | |
|---|---|---|---|---|---|---|
| | Baseline | Shock | Lactated Ringers | TgHb | Lactated Ringers | TgHb |
| Hct (%) | 46 ± 2 | 30 ± 1 ^{†} | 25 ± 1 ^{†} | 23 ± 3 ^{†} | 23 ± 1 ^{†} | 22 ± 2 ^{†} |
| tHb (g/dL) | 14.8 ± 0.4 | 9.8 ± 0.9 ^{†} | 8.2 ± 0.4 ^{†} | 9.47 ± 0.59 ^{†} | 8.43 ± 0.22 ^{†} | 8.88 ± 0.51 ^{†} |
| MAP (mmHg) | 116.49 ± 11.24 | 48.58 ± 11.75 ^{†} | 71.38 ± 12.87 ^{†‡} | 107.26 ± 8.68 | 63.23 ± 21.26 ^{†‡} | 106.54 ± 5.71 |
| HR (bpm) | 407.56 ± 43.61 | 374.50 ± 73.75 | 406.97 ± 63.13 | 407.75 ± 44.77 | 372.00 ± 63.07 | 427.27 ± 25.56 |
| pO₂ (mmHg) | 60.9 ± 2.1 | 100.8 ± 7.0 ^{†} | 109.7 ± 18.5 ^{†} | 80.3 ± 21.0 | 103.2 ± 19.2 ^{†} | 85.8 ± 14.5 |
| pCO₂ (mmHg) | 58.7 ± 2.3 | 45.7 ± 5.2 ^{†} | 33.4 ± 9.0 ^{†} | 49.6 ± 8.6 | 46.9 ± 16.4 | 47.7 ± 6.9 |
| Arterial pH | 7.366 ± 0.010 | 7.242 ± 0.015 ^{†} | 7.265 ± 0.146 ^{†} | 7.311 ± 0.104 | 7.484 ± 0.385 | 7.387 ± 0.068 |
| cLac (mmol/L) | 1.22 ± 0.47 | 11.70 ± 0.53 ^{†} | 12.2 ± 7.3 ^{†‡} | 5.93 ± 4.12 | 11.6 ± 7.3 ^{†‡} | 2.85 ± 2.15 |

^{†} vs baseline
^{‡}vs TgHb

FIG. 17 illustrates the absolute values and the relative to baseline values of MAP and HR following the acute hemorrhage and volume replacement (resuscitation) protocols with lactated ringers or TgHb.

Total hemoglobin concentration (tHb) and hematocrit (Hct) were significantly lower than baseline compared to shock, 30-min and 60-min after resuscitation with both lactated ringers or TgHb following the acute hemorrhage and volume replacement protocol. MAP was significantly lower than baseline at shock following an acute hemorrhage. Following resuscitation (volume replacement) with lactated ringers the MAP was significantly lower after 30-min and 60-min compared to baseline. Following resuscitation with TgHb the MAP had no significance to baseline, but MAP was significantly higher after resuscitation with TgHb after 30-min and 60-min then resuscitation with lactated ringers. HR had no significance after baseline after acute hemorrhage (shock) or after resuscitation with either lactated ringers or TgHb. pO2 was significantly lower than baseline at shock following an acute hemorrhage. Following resuscitation (volume replacement) with lactated ringers the pO2 was significantly higher after 30-min and 60-min compared to baseline. Following resuscitation with TgHb the pO2 had no significance to baseline or to the pO2 after resuscitation with lactated ringers after 30-min and 60-min. pCO2 was significantly lower than baseline at shock following an acute hemorrhage. Following resuscitation (volume replacement) with lactated ringers the pCO2 was significantly higher after 30-min compared to baseline. Following resuscitation with TgHb the pCO2 had no significance to baseline or to the pCO2 after resuscitation with lactated ringers after 30-min and 60-min. Arterial pH was significantly lower than baseline at shock following an acute hemorrhage. Following resuscitation (volume replacement) with lactated ringers the arterial pH was significantly higher after 30-min compared to baseline. Following resuscitation with TgHb the arterial pH had no significance to baseline or to the arterial pH after resuscitation with lactated ringers after 30-min and 60-min. Concentration of lactate (cLac) was significantly higher than baseline at shock following an acute hemorrhage. Following resuscitation (volume replacement) with lactated ringers the cLac was significantly higher after 30-min and 60-min compared to baseline. Following resuscitation with TgHb the cLac had no significance to baseline after 30-min and 60-min, however, cLac was significantly lower than the levels recorded after resuscitation with lactated ringers after 30-min and 60-min.

### Microvascular Hemodynamics

FIG. 18 illustrates the microhemodynamics of the arterioles relative to baseline. The arterioles are separated between small arterioles (<50 μm) and large arterioles (>50 μm), A) small arterioles diameter, B) large arteriole diameter, C) small arteriole flow, and D) large arteriole flow.

The diameter of the small or large arterioles had no significant difference compared to baseline after an acute hemorrhage or between groups following resuscitation with lactated ringers or TgHb. The flow of the small and large arterioles after an acute hemorrhage recorded a significant drop compared to baseline. Furthermore, the small and the large arterioles following resuscitation with lactated ringers recorded a significant drop 30-min and 60-min after resuscitation compared to baseline and following resuscitation with TgHb.

FIG. 19 illustrates the microhemodynamics of the venules relative to baseline. The venules between 20≤x<80 μm were combined A) diameter of venules between 20≤x<80 μm and B) flow of venules between 20≤x<80 μm.

The diameter of venules between 20≤x<80 μm had no significant to baseline after an acute hemorrhage, but it was significant after resuscitation with lactated rings. The flow of venules between 20≤x<80 μm was significantly lower following an acute hemorrhage compared to baseline. Furthermore, the flow was significantly lower 30-min and 60-min following resuscitation with lactated ringers compared to resuscitation with TgHb.

### Functional Capillary Density

FIG. 20 illustrates the functional capillary density in Golden Syrian Hamsters after an acute hemorrhage and 30-min and 60-min after resuscitation with lactated ringers or TgHb.

The functional capillary density was significantly lower after an acute hemorrhage compared to baseline. Following 30-min and 60-min after resuscitation with lactated ringers the functional capillary density was significantly lower than baseline. Furthermore, 30-min and 60-min after resuscitation with lactated ringers the functional capillary density was significantly lower than resuscitation with TgHb.

### Oxygen Saturation

FIG. 21 illustrates oxygen saturation in the A) arterioles and B) venules in Golden Syrian Hamsters after resuscitation with lactated ringers or TgHb.

Table 15 illustrates the values of oxygen saturation from 10 minutes after resuscitation with lacated ringers or TgHb through 60 minutes with a recording every 5 minutes.

TABLE 15
Oxygen Saturation After Resusication

| | Venules | | Arterioles | |
|---|---|---|---|---|
| Time | Lactated Ringers | TgHb | Lactated Ringers | TgHb |
| 10 | 0.16 ± 0.06 | 0.18 ± 0.04 | 0.20 ± 0.08 | 0.24 ± 0.10 |
| 15 | 0.19 ± 0.09 | 0.29 ± 0.04 | 0.22 ± 0.08 | 0.21 ± 0.07 |
| 20 | 0.16 ± 0.06 | 0.31 ± 0.05 | 0.24 ± 0.06 | 0.25 ± 0.08 |
| 25 | 0.17 ± 0.07 | 0.23 ± 0.05 | 0.21 ± 0.08 | 0.30 ± 0.11 |
| 30 | 0.15 ± 0.08 | 0.31 ± 0.06 | 0.16 ± 0.04 | 0.23 ± 0.08 |
| 35 | 0.17 ± 0.10 | 0.21 ± 0.06 | 0.20 ± 0.07 | 0.29 ± 0.08 |
| 40 | 0.18 ± 0.08 | 0.29 ± 0.07 | 0.25 ± 0.10 | 0.27 ± 0.08 |
| 45 | 0.17 ± 0.08 | 0.27 ± 0.08 | 0.23 ± 0.08 | 0.31 ± 0.08 |
| 50 | 0.18 ± 0.09 | 0.26 ± 0.07 | 0.19 ± 0.08 | 0.26 ± 0.09 |
| 55 | 0.18 ± 0.08 | 0.28 ± 0.03 | 0.22 ± 0.08 | 0.26 ± 0.08 |
| 60 | 0.17 ± 0.08 | 0.29 ± 0.02 | 0.21 ± 0.06 | 0.24 ± 0.08 |

FIG. 22 illustrates the hyperspectral images at baseline, shock, 30-min and 60-min after resuscitation. The darker the red, the higher the oxygen saturation percentage, the darker the blue the lower the oxygen saturation percentage. The figure distributed between 0-1, 0 being zero percent of oxygen present and 1 being 100 percent of oxygen present.

### Heart Health

Following the conclusion of the experimental protocol the Golden Syrian Hamsters were sacrificed and the hearts were harvested and analyzed. The following cardiac inflammation and function markers were analyzed and represented on FIG. 23: A) Cardiac c-reactive protein (CRP), B) cardiac interleukin-6 (IL-6), C) cardiac interleukin-10 (IL-10), D) cardiac tumor necrosis factor alpha (TNF-α), E) cardiac monocyte chemoattractant proteion-1 (MCP-1) and F) cardiac Troponin proteins were quantified in the tissue.

Table 16 indicates levels of cardiac CRP, cardiac IL-6, cardiac IL-10, cardiac TNF-α, cardiac MCP-1 and cardiac Troponin proteins that can indicate one or more status of the cardiac function. Additionally, nine Golden Syrian Hamsters were sacrificed without undergoing any protocol to establish a control. Significant to sham (†), significant to HBOC-201 (‡), and significant to TgHb (&).

TABLE 16
Acute Hemorrhage and Volume Replacement

| | Sham | Ringers | TgHb |
|---|---|---|---|
| Cardiac CRP (mg/dL) | 110.2 ± 20.3 | 175.2 ± 15.6^{†} | 163.8 ± 16.4^{&} |
| Cardiac IL-6 (μg/mL) | 102.7 ± 23.8 | 152.8 ± 6.4 | 121.0 ± 23.7 |
| Cardiac IL-10 (μg/mL) | 84.0 ± 16.3 | 120.1 ± 32.9 | 101.8 ± 4.2 |
| Cardiac TNF-a (ng/mL) | 138.0 ± 37.5 | 251.6 ± 95.8 | 167.8 ± 16.2 |
| Cardiac MCP-1 (pg/mL) | 118.0 ± 31.0 | 208.4 ± 69.6 | 168.3 ± 10.2 |
| Cardiac | 144.7 ± 25.3 | 155.0 ± 16.2 | 137.3 ± 20.9 |
| Troponin (ng/mL) | | | |

^{†}vs sham,
‡ vs HBOC-201,
^{&}vs TgHb

Cardiac CRP was significantly elevated in the ringers and TgHb groups compared to sham. Cardiac IL-6 showed a significant increase in lactated ringers compared to sham, with TgHb showing no significance. Cardiac Il-1, cardiac TNF-α, cardiac MCP-1, and cardiac troponin showed no significance between the groups, however, the values for ringers seem to be elevated compared to sham and TgHb.

### Kidney Health

Upon completion of the experiment the Golden Syrian Hamsters were sacrificed where the kidney, urine and plasma were collected. FIG. 24 indicates A) plasma blood urea nitrogen (BUN), B) urine neutrophil gelatinase-associated lipocalin (NAGL), and C) urine creatinine, these measurements evaluate kidney function. Furthermore, three Golden Syrian Hamsters were sacrificed without undergoing any protocol to establish a control.

Table 17 indicates levels of plasma BUN, urine NGAL and urine creatinine proteins that can indicate one or more status of the kidney function. Significant to sham (†), significant to HBOC-201 (‡), and significant to TgHb (&)

TABLE 17
Acute Hemorrhage and Volume Replacement

| | Sham | Ringers | TgHb |
|---|---|---|---|
| Plasma | 1.1 ± 0.1 | 1.5 ± 0.2 | 1.6 ± 0.1 |
| BUN (mmol/L) | | | |
| Urine NAGL (ng/mL) | 476.8 ± 80.9 | 691.0 ± 68.6^{†&} | 512.5 ± 106.0 |
| Urine | 1.4 ± 0.3 | 3.0 ± 0.5^{†&} | 1.4 ± 0.2 |
| Creatinine (mg/dL) | | | |

^{†}vs sham,
‡ vs HBOC-201,
^{&}vs TgHb

Plasma BUN levels were not significantly different between all three groups. Furthermore, urine NAGL levels were significantly higher in the ringers group compared to both lactated ringers and TgHb. Urine creatinine levels were significantly higher in the ringers group compared to sham and TgHb.

Kidney inflammatory markers (FIG. 25) were quantified by A) renal IL-6, B) renal IL-1, C) renal CXCL1 and D) renal IL-10. Furthermore, three Golden Syrian Hamsters were sacrificed without undergoing any protocol to establish a control.

Table 18 indicates levels of renal IL-6, renal IL-1, renal CXCL1 and renal IL-10 proteins that can indicate one or more status of the kidney inflammation. Also, nine Golden Syrian Hamsters were sacrificed without undergoing any protocol to establish a control. Significant to sham (†), significant to Ringers (‡), and significant to TgHb (&).

TABLE 18
Acute Hemorrhage and Volume Replacement

| | Sham | Ringers | TgHb |
|---|---|---|---|
| Renal IL-6 (μg/mL) | 128.5 ± 9.5 | 234.8 ± 57.5^{†} | 228.6 ± 37.2^{†} |
| Renal IL-1 (μg/mL) | 120.5 ± 10.1 | 200.4 ± 73.1 | 137.6 ± 15.5 |
| Renal CLCX1 (μg/mL) | 150.8 ± 26.7 | 299.4 ± 49.2^{&} | 147.2 ± 14.9 |
| Renal IL-10 (μg/mL) | 120.8 ± 32.1 | 194.7 ± 60.0 | 161.0 ± 19.6 |

^{†}vs sham,
‡ vs HBOC-201,
^{&}vs TgHb

Renal IL-6 had a significant increase in the ringers group and the TgHb group when compared to sham. Renal CXCL-1 showed a significant increase in the ringers' group compared to the TgHb group. Lastly, renal IL-1 and renal IL-10 showed no significant differences between the three groups.

### Liver Health

Upon completion of the experiment A) Hepatic CXCL1, B) plasma aspartate transaminase (AST), and C) plasma alanine transaminase (ALT) was measured to evaluate liver function and inflammation of the liver, results are shown in FIG. 26.

Table 19 indicates levels of hepatic CXCL1, plasma AST and plasma ALT proteins that can indicate one or more status of the liver inflammation and liver function. Furthermore, three Golden Syrian Hamsters were sacrificed without undergoing any protocol to establish a control. Significant to sham (†), significant to HBOC-201 (‡), and significant to TgHb (&).

TABLE 19
Acute Hemorrhage and Volume Replacement

| | Sham | Ringers | TgHb |
|---|---|---|---|
| Hepatic CXCL1 (μg/mL) | 107.5 ± 14.2 | 345.5 ± 125.3^{†&} | 179.0 ± 20.2 |
| Plasma AST (IU/L) | 30.4 ± 3.2 | 37.8 ± 12.2 | 35.0 ± 1.9 |
| Plasma ALT (IU/L) | 33.2 ± 9.6 | 40.5 ± 9.8 | 39.5 ± 4.6 |

^{†}vs sham,
‡ vs HBOC-201,
^{&}vs TgHb

Hepatic CXCL1 levels were significantly higher in the ringers' group when compared to the sham and TgHb. Plasma AST levels and plasma ALT levels showed no significant differences between the three groups, however the levels of both AST and ALT were higher in the ringers group compared to TgHb and sham.

### Vasculature Health

Following the conclusion of the experiment plasma was collected, A) plasma IL-6, B) plasma CXCL1, and C) plasma IL-10 were quantified in FIG. 27.

Table 20 indicates levels of plasma IL-6, plasma CXCL1, and plasma IL-10 proteins that can indicate one or more status of liver inflammation and liver function. Also, three Golden Syrian Hamsters were sacrificed without undergoing any protocol to establish a control. Significant to sham (†), significant to HBOC-201 (‡), and significant to TgHb (&).

TABLE 20
Acute Hemorrhage and Volume Replacement

| | Sham | Ringers | TgHb |
|---|---|---|---|
| Plasma IL-6 (μg/mL) | 73.0 ± 5.7 | 154.1 ± 19.7^{†} | 198.2 ± 11.3^{†‡} |
| Plasma CLCX1 (μg/mL) | 144.9 ± 22.2 | 229.9 ± 65.9 | 240.2 ± 43.5 |
| Plasma IL-10 (μg/mL) | 94.6 ± 9.8 | 123.6 ± 22.1^{†} | 159.3 ± 7.7^{†} |

^{†}vs sham,
^{‡}vs HBOC-201,
& vs TgHb

Plasma IL-6 levels were significantly higher compared to the sham in both the ringers and TgHb groups. Furthermore, plasma IL-6 levels were significantly higher following the infusion of TgHb compared to the ringers' group. Plasma CXCL1 levels showed no significant difference between the three groups. Plasma IL-10 levels were significantly higher in the TgHb group when compared to sham and the group with the infusion of ringers.

### Ferritin and Bilirubin Concentration

Following the conclusion of the experiment the ferritin and bilirubin concentration was quantified in the plasma, heart, spleen, liver, kidney and the ferritin concentration was quantified. FIG. 28 indicates A) plasma ferritin, B) cardiac ferritin, C) splenic ferritin D) hepatic ferritin, E) renal ferritin, F) plasma bilirubin which can be speculated to be associated with levels of iron in the circulation.

Table 21 indicates levels of plasma ferritin, cardiac ferritin, splenic ferritin, hepatic ferritin, renal ferritin, and plasma bilirubin proteins that can indicate iron concentration and levels of hemolysis in the vasculature and vital organs. Also, three Golden Syrian Hamsters were sacrificed without undergoing any protocol to establish a control. Significant to sham (†), significant to HBOC-201 (‡), and significant to TgHb (&).

TABLE 21
Acute Hemorrhage and Volume Replacement

| | Sham | Ringers | TgHb |
|---|---|---|---|
| Plasma Ferritin (μg/L) | 269.3 ± 37.3 | 166.6 ± 36.1^{&} | 267.0 ± 25.5 |
| Cardiac Ferritin (ng/L) | 333.4 ± 37.5 | 161.2 ± 18.4^{†&} | 250.4 ± 34.5 |
| Splenic Ferritin (μg/L) | 399.9 ± 86.6 | 342.6 ± 41.0 | 298.7 ± 41.2 |
| Hepatic Ferritin (μg/L) | 339.0 ± 28.6 | 355.1 ± 40.7 | 347.3 ± 37.8 |
| Renal Ferritin (μg/L) | 189.1 ± 24.6 | 320.0 ± 62.4 | 217.4 ± 34.8 |
| Plasma Bilirubin | 2.8 ± 0.4 | 2.0 ± 0.3^{†&} | 2.7 ± 0.2 |
| (μmol/L) | | | |

^{†}vs sham,
‡ vs HBOC-201,
^{&}vs TgHb

Plasma ferritin levels were significantly higher in the TgHb group compared to ringers'; however, the rest of the group comparisons showed no significant difference between each other. There was no significant difference in splenic, hepatic, and renal ferritin levels compared to sham or between groups. Cardiac ferritin levels were significantly higher in both TgHb and sham when compared to the ringer's group. Moreover, the cardiac ferritin was also significantly higher for the TgHb when compared to the sham. Lastly, plasma bilirubin levels were significantly higher in both TgHb and sham when compared to the ringer's group.

### Catecholamines

Following the conclusion of the experiment the plasma epinephrine and plasma norepinephrine concentration was quantified in the plasma. FIG. 29 indicates A) plasma epinephrine and B) plasma norepinephrine which can be speculated to be associated with levels of the nervous system.

Table 22 indicates levels of plasma epinephrine and plasma norepinephrine proteins that can indicate iron concentration and levels of nervous system reactions. Also, three Golden Syrian Hamsters were sacrificed without undergoing any protocol to establish a control. Significant to sham (†), significant to HBOC-201 (‡), and significant to TgHb (&).

TABLE 22
Acute Hemorrhage and Volume Replacement

| | Sham | Ringers | TgHb |
|---|---|---|---|
| Plasma Epinephrine | 320.8 ± 38.0 | 613.9 ± 75.5^{†&} | 289.1 ± 32.1 |
| (ng/mL) | | | |
| Plasma Norepinephrine | 241.4 ± 73.4 | 445.1 ± 32.3^{†&} | 209.2 ± 22.3 |
| (ng/mL) | | | |

^{†}vs sham,
‡ vs HBOC-201,
^{&}vs TgHb

Both epinephrine and norepinephrine levels showed a significant increase following the infusion of ringers compared to sham and to the group infused with TgHb.

Characterization of Fully Oxygenated Hemoglobin (R-State) and Fully Deoxygenated Hemoglobin (T-State) Polymerized with Transglutaminase

### Materials and Methods

### Hb Preparation

Hb (P68871, P69905) for this study was prepared via tangential flow filtration as previously described. (Palmer, A. F.; Sun, G.; Harris, D. R. Tangential Flow Filtration of Hemoglobin. Biotechnol. Prog. 2009, 25 (1), 189-199. https://doi.org/10.1002/btpr.119). The concentration of Hb was determined spectrophotometrically.

### Polymerization of Hb Using Transglutaminase

Hemoglobin protein dispersions were prepared at either 20% solids content (w/v) and adjusted to pH 7.5. The protein substrate was mixed with microbial transglutaminase at a ratio of approximately 5 units per gram of hemoglobin and slowly agitated in a rotating incubator at 37° C. At timed intervals, samples were taken for SDS-PAGE and OPA analyses. In most cases, the soluble supernatants obtained after centrifugation at 11950 g were tested. Residual protein content in transglutaminase-treated samples that were kept at 37° C. for 24 hours was measured using the BCA method.

### Rheological Measurements

The viscosity was measured for transglutaminase polymerization human R-State (fully oxygenated) hemoglobin and transglutaminase polymerization human T-State (fully deoxygenated) hemoglobin. The viscosity was measured using a computerized cone-plate rheometer at 37° C. (Discovery HR-2; TA Instruments, New Castle, DE).

### Window Chamber Preparation in Golden Syrian Hamsters

Investigations were performed in 55-65 g male Golden Syrian Hamsters (Charles River Laboratories, Boston, MA) fitted with a dorsal skinfold window chamber. Animal handling and care followed the NIH Guide for the Care and Use of Laboratory Animals. The experimental protocol was approved by the local animal care committee. The hamster window chamber model is widely used for microvascular studies in the unanesthetized state, and the complete surgical technique is described in detail elsewhere. (Colantuoni, A.; Bertuglia, S.; Intaglietta, M. Quantitation of Rhythmic Diameter Changes in Arterial Microcirculation. Am. J. Physiol. 1984, 246 (4 Pt 2), H508-517. https://doi.org/10.1152/ajpheart.1984.246.4.H508.) The experimental animal was allowed at least 2 days for recovery before the preparation was assessed under the microscope for any signs of edema, bleeding, or unusual neovascularization. Animals were anesthetized again, and arterial and venous catheters filled with a heparinized saline solution (30 IU/ml) were implanted. Catheters were tunneled under the skin, exteriorized at the dorsal side of the neck, and securely attached to the window frame. The microvasculature was examined 3 to 4 days after the initial surgery and only animals with window chambers whose tissue did not present regions of low perfusion, inflammation, and edema were entered into the study. (Tsai, A. G.; Friesenecker, B.; McCarthy, M.; Sakai, H.; Intaglietta, M. Plasma Viscosity Regulates Capillary Perfusion during Extreme Hemodilution in Hamster Skinfold Model. Am. J. Physiol. 1998, 275 (6), H2170-2180)

### Inclusion Criteria

Animals were considered suitable for experiments if systemic parameters were as follows: heart rate (HR)>340 beats/min, mean arterial blood pressure (MAP)>80 mm Hg, systemic hematocrit (Hct)>45%, and arterial O2 partial pressure (pAO2)>50 mm Hg. Additionally, animals with signs of low perfusion, inflammation, edema, or bleeding in their microvasculature were excluded from the study.

### Experimental Setup

The unanesthetized animal was placed in a restraining tube with a longitudinal slit from which the window chamber protruded, then fixed to the microscopic stage for transillumination with the intravital microscope (BX51WI, Olympus, Japan). Animals were given 20 minutes to adjust to the tube environment and images were obtained using a CCD camera (4815, COHU, San Diego, CA). Measurements were carried out using a 40× (LUMPFL-WIR, numerical aperture 0.8, Olympus) water immersion objective.

### Systemic Parameters

The Mean Arterial Pressure (MAP) and Heart Rate (HR) were monitored continuously (MP150, Biopac System Inc., Santa Barbara, CA). Hematocrit (Hct) was measured from centrifuged arterial blood samples taken in heparinized capillary tubes. Hb content was determined spectrophotometrically (B-Hemoglobin, Hemocue, Stockholm, Sweden). Arterial blood was collected in heparinized glass capillaries (50 μL) and immediately analyzed for total hemoglobin concentration (tHb), partial pressure of oxygen (pO2), partial pressure of carbon dioxide (pCO2), and pH (ABL90; Radiometer America, Brea, CA).

### Microhemodynamics

Arteriolar and venular blood flow velocities were measured online by using the photodiode cross-correlation method (Intaglietta, M.; Silverman, N. R.; Tompkins, W. R. Capillary Flow Velocity Measurements in Vivo and in Situ by Television Methods. Microvasc. Res. 1975, 10 (2), 165-179. https://doi.org/10.1016/0026-2862 (75) 90004-7) (Photo Diode/Velocity Tracker Model 102B, Vista Electronics, San Diego, CA). The measured centerline velocity (V) was corrected according to vessel size to obtain the mean RBC velocity. (Lipowsky, H. H.; Zweifach, B. W. Application of the “Two-Slit” Photometric Technique to the Measurement of Microvascular Volumetric Flow Rates. Microvasc. Res. 1978, 15 (1), 93-101. https://doi.org/10.1016/0026-2862 (78) 90009-2.) A video image-shearing method was used to measure vessel diameter (D). (Intaglietta, M.; Tompkins, W. R. Microvascular Measurements by Video Image Shearing and Splitting. Microvasc. Res. 1973, 5 (3), 309-312. (2) Lipowsky, H. H.; Zweifach, B. W. Application of the “Two-Slit” Photometric Technique to the Measurement of Microvascular Volumetric Flow Rates. Microvasc. Res. 1978, 15 (1), 93-101. https://doi.org/10.1016/0026-2862 (78) 90009-2.) Blood flow (Q) was calculated from the values measured as Q=π×V (D/2)\hat{ }2. Changes in arteriolar and venular diameter from baseline were used as indicators of a change in vascular tone. This calculation assumes a parabolic velocity profile and has been found to be applicable to tubes of 15-100 μm internal diameters and for Hets in the range of 6-60%. (Lipowsky, H. H.; Zweifach, B. W. Application of the “Two-Slit” Photometric Technique to the Measurement of Microvascular Volumetric Flow Rates. Microvasc. Res. 1978, 15 (1), 93-101. https://doi.org/10.1016/0026-2862 (78) 90009-2)

### Functional Capillary Density (FCD)

Functional capillaries, defined as capillary segments that have transit of at least one RBC in a 60 second period in 10 successive microscopic fields, were assessed in a region of 0.46 mm². The FCD (cm-1) was calculated as the total length of RBC perfused capillaries divided by the viewing area (0.46 mm²)

### Hypervolemic Infusion (Top-Load) Protocol

Hamsters received a hypervolemic infusion as a bolus through the carotid artery of 10% of the hamster's blood volume. The hamsters blood volume was calculated as 7% of their total body weight. The experimental groups were labeled based on the solutions R-state (fully oxygenated) and T-State (fully deoxygenated) TgHb all solutions were kept at a concentration of 100 mg/mL. After each infusion, animals were allowed 20 minutes to stabilize before systemic and microvascular characterization. The experimental timeline is described in FIG. 30.

### Pharmacokinetics and Oxidation Rate Measurements

Pharmacokinetics and oxidation rate was studied for 48 hours after exchange transfusion with 100 mg/mL R-State or T-State TgHb. Briefly, hamsters fitted with the dorsal window chamber were exchanged with 40% of the estimated BV (7% of body weight). Test solutions were infused into the jugular vein catheter at a rate of 100 μl/min with simultaneous blood withdrawal at the same rate from the carotid artery catheter via a dual syringe pump (Harvard Apparatus, Holliston, MA). Blood samples (50 μL) were taken after exchange transfusion at 0.5, 1, 2, 8, 12, 24, and 48 hours. Pharmacokinetic and oxidation rates were determined using non-compart-mental analysis.

### Organ Harvesting and Analysis for Markers of Organ Damage and Inflammation.

Blood was collected and centrifuged to separate the plasma. Animals were sacrificed with Fatal Plus (sodium pentobarbital, 300 mg/kg), urine was collected, and heart, kidneys, liver, and spleen were harvested. Markers of inflammation, function, and organ injury were evaluated using the ELISA Kits/Assays described in Table 1.

Table 1 describes the ELISA Kits/Assays used to quantify markers of organ damage and inflammation.

### Statistical Analysis

Results are presented as mean±standard deviation. Data within each group was analyzed using analysis of variance for repeated measurements (ANOVA, Kruskal-Wallis test). When appropriate, post hoc analyses were performed with the Dunns multiple comparison test. Microhemodynamic measurements were compared to baseline levels obtained before the experimental procedure. The box-whisker plot separates the data into quartiles, with the top of the box defining the 75th percentile, the line within the box giving the median, and the bottom of the box showing the 25th percentile. The upper “whisker” defines the 95th percentile and the lower whisker defines the 5th percentile. Microhemodynamic data is presented as absolute values and ratios relative to baseline values. A ratio of 1.0 signifies no change from baseline while lower and higher ratios are indicative of changes proportionally lower and higher than baseline (i.e., 1.5 would mean a 50% increase from the baseline level). The same vessels and capillary fields were followed so that direct comparisons to their baseline levels could be performed, allowing for more robust statistics for small sample populations. All statistics were calculated using GraphPad Prism 4.01 (GraphPad Software, Inc., San Diego, CA). Changes were considered statistically significant if p<0.05

### Results

### Mean Arterial Pressure (MAP), Heart Rate (HR), Hematological Properties and Blood Gases

Table 23 represents the baseline values and the measured quantities of total hemoglobin concentration (tHb), hematocrit (Hct), mean arterial pressure (MAP), heart rate (HR), pH, pCO2, pO2, after the hypervolemic infusion of R-State or T-State TgHb.

TABLE 23
20 Minutes After Hypovolemic Infusion

| | Baseline | R-State | T-State |
|---|---|---|---|
| tHb (g/dL) | 15.5 ± 0.4 | 15.5 ± 0.9 | 15.4 ± 0.1 |
| Hct (%) | 47 ± 1 | 47 ± 2 | 45 ± 2 |
| MAP (mmHg) | 105.18 ± 6.10 | 111.98 ± 19.68 | 111.27 ± 8.04 |
| HR (bpm) | 430.22 ± 5.08 | 423.58 ± 21.27 | 415.30 ± 25.02 |
| pH | 7.36 ± 0.01 | 7.39 ± 0.03 | 7.37 ± 0.06 |
| pCO₂ (mmHg) | 57.2 ± 1.6 | 56.6 ± 3.2 | 52.6 ± 4.6 |
| pO₂ (mmHg) | 63.5 ± 1.9 | 60.0 ± 8.1 | 63.7 ± 10.5 |
| pHb (g/dL) | — | 1.1 ± 0.3 | 1.0 ± 0.1 |

FIG. 31 indicates results of MAP and HR with their relative to baseline following the hypervolemic infusion of R-State or T-State TgHb.

Total hemoglobin concentration (tlb), hematocrit (Hct), mean arterial pressure (MAP), heart rate (HR), pH, partial arterial pressure of carbon dioxide (pCO2), and partial arterial pressure of oxygen (pO2) had no significance compared to baseline or between groups after a hypovolemic infusion of R-State or T-State TgHb.

### Biophysical Properties

Table 24 indicates the values of the viscosity and the shear stress of R-State TgHb (10 g/dL) at shear rates from 0 to 1000.

TABLE 24

| Shear rate | Viscosity | Step time | Normal stress | Stress | Temperature | Viscosity |
|---|---|---|---|---|---|---|
| (s⁻¹) | (cP) | (s) | (Pa) | (Pa) | (° C.) | (Pa · s) |
| 1000 | 19.1235 | 23.3631 | −189.48 | 19.1235 | 37.011 | 0.019124 |
| 794.328 | 20.6738 | 49.5819 | −179.625 | 16.4218 | 36.996 | 0.020674 |
| 630.958 | 22.3187 | 70.6904 | −170.819 | 14.0822 | 37.011 | 0.022319 |
| 501.189 | 24.2025 | 91.788 | −169.53 | 12.13 | 37.019 | 0.024203 |
| 398.108 | 26.2503 | 112.879 | −168.268 | 10.4504 | 37.002 | 0.02625 |
| 316.228 | 28.4109 | 139.003 | −170.143 | 8.98431 | 37.004 | 0.028411 |
| 251.189 | 30.7695 | 165.174 | −171.219 | 7.72896 | 37 | 0.03077 |
| 199.526 | 33.3175 | 186.297 | −172.758 | 6.64771 | 37.012 | 0.033318 |
| 158.49 | 36.0835 | 207.416 | −174.687 | 5.71886 | 36.997 | 0.036084 |
| 125.893 | 39.1675 | 228.485 | −176.75 | 4.93092 | 37.006 | 0.039168 |
| 99.9999 | 42.473 | 249.591 | −179.212 | 4.24729 | 36.99 | 0.042473 |
| 79.4328 | 46.1242 | 270.707 | −180.122 | 3.66378 | 37.013 | 0.046124 |
| 63.0959 | 50.1946 | 291.777 | −181.841 | 3.16707 | 37.02 | 0.050195 |
| 50.1188 | 54.7351 | 312.849 | −183.64 | 2.74325 | 37.001 | 0.054735 |
| 39.8106 | 59.9188 | 333.942 | −184.528 | 2.3854 | 37.003 | 0.059919 |
| 31.6226 | 65.7563 | 355.081 | −187.367 | 2.07939 | 36.993 | 0.065756 |
| 25.1189 | 72.2917 | 376.206 | −187.473 | 1.81589 | 37.005 | 0.072292 |
| 19.9525 | 79.7549 | 397.336 | −189.082 | 1.59131 | 36.987 | 0.079755 |
| 15.849 | 88.5512 | 418.456 | −190.803 | 1.40345 | 37 | 0.088551 |
| 12.5892 | 99.1661 | 439.539 | −190.559 | 1.24842 | 37.009 | 0.099166 |
| 9.99993 | 111.37 | 460.611 | −193.188 | 1.11369 | 37.002 | 0.11137 |
| 7.94321 | 125.721 | 481.704 | −192.712 | 0.998629 | 37.011 | 0.125721 |
| 6.30959 | 143.294 | 502.788 | −193.306 | 0.904128 | 36.986 | 0.143294 |
| 5.01176 | 163.231 | 523.872 | −195.42 | 0.818074 | 36.99 | 0.163231 |
| 3.98104 | 188.572 | 544.95 | −194.315 | 0.75071 | 37.002 | 0.188572 |
| 3.16209 | 216.967 | 566.059 | −197.335 | 0.686069 | 36.992 | 0.216967 |
| 2.61191 | 253.822 | 587.179 | −196.198 | 0.637578 | 36.998 | 0.253822 |
| 1.99525 | 298.343 | 608.318 | −195.579 | 0.595268 | 37.002 | 0.298343 |
| 1.58483 | 350.272 | 629.463 | −196.289 | 0.55512 | 37.008 | 0.350272 |
| 1.259 | 410.773 | 650.597 | −199.97 | 0.517163 | 36.998 | 0.410773 |
| 1.00004 | 488.012 | 671.747 | −200.757 | 0.488031 | 36.995 | 0.488012 |

Table 25 indicates the values of the viscosity and the shear stress of T-State TgHb (10 g/dL) at shear rates from 0 to 1000.

TABLE 25

| Shear rate | Viscosity | Step time | Stress | Temperature | Viscosity | Shear rate |
|---|---|---|---|---|---|---|
| (s⁻¹) | (cP) | (s) | (Pa) | (° C.) | (Pa · s) | (s⁻¹) |
| 1000 | 7.20346 | 26.3742 | −148.385 | 7.20347 | 36.997 | 0.007203 |
| 794.328 | 7.49098 | 52.5081 | −135.177 | 5.95029 | 37.001 | 0.007491 |
| 630.958 | 7.79926 | 76.6922 | −123.767 | 4.92101 | 37.011 | 0.007799 |
| 501.188 | 8.14202 | 104.765 | −116.955 | 4.08068 | 37 | 0.008142 |
| 398.107 | 8.56164 | 125.833 | −113.545 | 3.40844 | 36.997 | 0.008562 |
| 316.229 | 9.04731 | 151.953 | −112.713 | 2.86102 | 36.991 | 0.009047 |
| 251.189 | 9.60024 | 178.154 | −111.673 | 2.41147 | 37.003 | 0.0096 |
| 199.526 | 10.2444 | 204.276 | −110.729 | 2.04403 | 36.995 | 0.010244 |
| 158.489 | 11.0106 | 230.46 | −111.805 | 1.74506 | 36.997 | 0.011011 |
| 125.893 | 11.9351 | 256.559 | −114.854 | 1.50254 | 37.002 | 0.011935 |
| 100 | 13.0385 | 277.638 | −114.11 | 1.30386 | 37.014 | 0.013039 |
| 79.4326 | 14.4005 | 298.693 | −114.857 | 1.14387 | 37.006 | 0.014401 |
| 63.0956 | 16.0625 | 319.75 | −114.915 | 1.01347 | 36.997 | 0.016063 |
| 50.1188 | 18.0864 | 340.812 | −116.733 | 0.90647 | 36.994 | 0.018086 |
| 39.8107 | 20.5627 | 361.888 | −117.879 | 0.818616 | 37.004 | 0.020563 |
| 31.6229 | 23.6055 | 383.033 | −118.183 | 0.746473 | 36.999 | 0.023606 |
| 25.1189 | 27.3855 | 404.181 | −120.205 | 0.687894 | 37.001 | 0.027386 |
| 19.9527 | 31.9594 | 425.33 | −121.318 | 0.637677 | 37.001 | 0.031959 |
| 15.8489 | 37.6496 | 446.439 | −122.167 | 0.596704 | 37.005 | 0.03765 |
| 12.5892 | 44.575 | 467.572 | −120.727 | 0.561166 | 37.007 | 0.044575 |
| 10.0001 | 53.0565 | 488.68 | −122.447 | 0.530572 | 37.008 | 0.053057 |
| 7.94328 | 63.5057 | 509.766 | −123.776 | 0.504443 | 37.012 | 0.063506 |
| 6.30953 | 76.3197 | 530.8 | −122.841 | 0.481542 | 37.01 | 0.07632 |
| 5.01207 | 91.9617 | 551.919 | −125.628 | 0.460918 | 36.996 | 0.091962 |
| 3.98106 | 111.358 | 573.02 | −123.439 | 0.443323 | 37.001 | 0.111358 |
| 3.16227 | 134.984 | 594.172 | −125.628 | 0.426855 | 37.001 | 0.134984 |
| 2.51189 | 163.986 | 615.33 | −124.09 | 0.411916 | 37.006 | 0.163986 |
| 1.99519 | 199.637 | 636.478 | −124.926 | 0.398314 | 37 | 0.199637 |
| 1.58492 | 242.791 | 657.579 | −125.541 | 0.384804 | 36.997 | 0.242791 |
| 1.25909 | 294.909 | 678.66 | −129.096 | 0.371316 | 36.996 | 0.294909 |
| 0.999938 | 359.548 | 699.742 | −128.575 | 0.359526 | 37.004 | 0.359548 |

FIG. 32 indicates the shear rate and shear stress of both R-State and T-State TgHb.

FIG. 33 indicates the slog (shear rate) and log (viscosity) of both R-State and T-State TgHb.

Table 26 indicates the values of the transglutaminase to hemoglobin ratio, concentration of TgHb, metHb percentage, viscosity at 100 s⁻¹, and half-life in hours.

TABLE 26
Biophysical Properties of R state and T State of TgHb.

| | R-State | T-State |
|---|---|---|
| Transglutamase:Hb Raio | 5:1 | 5:1 |
| Concentration (g/dL) | 10 | 10 |
| MetHb (%) | 5 | 5 |
| Viscosity (cP) | 42 | 13 |
| Half Life (Hrs) | 26 | 17 |

* Viscosity Measured at 100 s⁻¹

### Pharmacokinetics and Oxidation Rate

Table 27 illustrates plasma Hb and plasma methHb fraction percentages over time from 0 hours to 48 hours of R-State and T-State TgHb.

TABLE 27
Pharmacokinetics and Oxidation Rate

| | R-State | | T-State | |
|---|---|---|---|---|
| Time (hours) | Plasma Hb (g/dL) | Plasma metHb (%) | Plasma Hb (g/dL) | Plasma metHb (%) |
| 0 | 1.13 ± 0.09 | 0.04 ± 0.01 | 1.27 ± 0.09 | 0.05 ± 0.01 |
| 2 | 0.94 ± 0.16 | 0.05 ± 0.01 | 1.10 ± 0.19 | 0.05 ± 0.01 |
| 4 | 1.09 ± 0.17 | 0.05 ± 0.01 | 0.97 ± 0.16 | 0.06 ± 0.01 |
| 8 | 0.85 ± 0.09 | 0.05 ± 0.01 | 1.01 ± 0.23 | 0.07 ± 0.01 |
| 12 | 0.80 ± 0.11 | 0.06 ± 0.01 | 0.82 ± 0.16 | 0.09 ± 0.01 |
| 24 | 0.53 ± 0.09 | 0.08 ± 0.01 | 0.58 ± 0.10 | 0.11 ± 0.01 |
| 48 | 0.25 ± 0.05 | 0.13 ± 0.01 | 0.35 ± 0.04 | 0.17 ± 0.01 |

FIG. 34 illustrates A) plasma Hb and B) plasma methHb fraction percentages over time from 0 hours to 48 hours of R-State and T-State TgHb.

### Microvascular Hemodynamics

FIG. 35 indicates results of diameter and flow of the arterioles and venules in the microcirculation relative to their baseline following the hypervolemic infusion protocol described above. The microhemodynamics include the diameter and flow of arterioles ranging from 20 μm-100 μm. The arterioles were segregated by diameter into two groups A, D)≤50 μm (small art), and B, E)>50 μm (large art). The venules were sorted by diameter between C, F) 20 μm-100 μm.

The diameter of small arterioles had no significance to baseline or between groups after hypovolemic infusion of R-State and T-State TgHb. The flow of small arterioles was significantly lower than baseline following a hypovolemic infusion of R-State or T-State TgHb. The diameter of large arterioles was significantly lower than baseline after a hypovolemic infusion of R-State or T-State TgHb. The flow of large arterioles was significantly lower than baseline after a hypovolemic infusion of R-State or T-State TgHb. Furthermore, following a hypovolemic infusion of R-State TgHb it was significantly lower than a hypovolemic infusion of T-State TgHb. The diameter of the venules had no significance to baseline or between groups after hypovolemic infusion of R-State or T-State TgHb. The flow of the venules was significantly lower than baseline following a hypovolemic infusion of R-State or T-State TgHb.

### Example 4: Functional Capillary Density

The number of functional capillaries was quantified 20 minutes after hypervolemic infusion protocol described above. FIG. 36 indicates results of functional capillary density in Golden Syrian hamsters after the hypervolemic infusion of R-State or T-State TgHb.

The FCD following a hypovolemic infusion of T-State TgHb was significantly lower than baseline.

### Example 5; Heart Health

Following the conclusion of the hypervolemic infusion protocol the Golden Syrian Hamsters were sacrificed and the heart of each sacrificed hamster was analyzed. FIG. 37 portrays A) Cardiac c-reactive protein (CRP), B) cardiac interleukin-6 (IL-6), C) cardiac interleukin-10 (IL-10), D) cardiac tumor necrosis factor alpha (TNF-α), E) cardiac monocyte chemoattractant proteion-1 (MCP-1) and F) cardiac Troponin in the tissue to evaluate cardiac inflammation and function.

Table 28 indicates levels of cardiac CRP, cardiac IL-6, cardiac IL-10, cardiac TNF-α, cardiac MCP-1 and cardiac Troponin proteins that can indicate one or more status of the cardiac function. Also, three Golden Syrian Hamsters were sacrificed without undergoing any protocol to establish a control. Significant to sham (†), significant to R-State ($), and significant to T-State (&).

TABLE 28
20 Minutes After Hypovolemic Infusion

| | Sham | R-State | T-State |
|---|---|---|---|
| Cardiac CRP (mg/dL) | 106.3 ± 27.9 | 169.9 ± 52.6 | 209.5 ± 27.6^{†} |
| Cardiac IL-6 (μg/mL) | 101.5 ± 32.5 | 137.3 ± 23.1 | 148.8 ± 32.9 |
| Cardiac IL-10 (μg/mL) | 84.4 ± 20.3 | 105.1 ± 8.5 | 136.8 ± 15.8^{†‡} |
| Cardiac TNF-a (ng/mL) | 146.6 ± 36.3 | 178.4 ± 8.0 | 223.9 ± 20.2^{†‡} |
| Cardiac MCP-1 (pg/mL) | 124.1 ± 44.3 | 180.6 ± 16.9^{†} | 239.6 ± 35.6^{†‡} |
| Cardiac Troponin | 141.5 ± 27.8 | 169.2 ± 29.1 | 158.0 ± 23.6 |
| (ng/mL) | | | |

^{†}vs sham,
^{‡}vs R-State,
& vs T-State

Cardiac CRP were significantly higher following a hypovolemic infusion of T-State TgHb compared to sham. Cardiac IL-6, and cardiac troponin had no significance between groups or to sham after a hypovolemic infusion of R-State or T-State TgHb. Cardiac IL-1 and cardiac TNF-α levels were significantly higher than sham following a hypovolemic infusion of R-State or T-State TgHb. Cardiac MCP-1 levels were significantly higher compared to sham following a hypovolemic infusion of R-State and T-State TgHb. Furthermore, following a hypovolemic infusion of T-State TgHb the MCP-1 levels were significantly higher than MCP-1 levels following a hypovolemic infusion of R-State TgHb.

### Kidney Health

Upon completion of the experiment the Golden Syrian Hamsters were sacrificed where the kidney, urine and plasma were collected. FIG. 38 portray A) plasma blood urea nitrogen BUN, B) urine neutrophil gelatinase-associated lipocalin NAGL, and C) urine creatinine these were measured to evaluate kidney function.

Table 29 indicates levels of plasma BUN, urine NGAL and urine creatinine proteins that can indicate one or more status of the kidney function. Also, three Golden Syrian Hamsters were sacrificed without undergoing any protocol to establish a control. Significant to sham (†), significant to R-State ($), and significant to T-State (&).

TABLE 29
20 Minutes After Hypovolemic Infusion

| | Sham | R-State | T-State |
|---|---|---|---|
| Plasma BUN | 1.1 ± 0.2 | 1.9 ± 0.2^{†} | 1.8 ± 0.1 |
| (mmol/L) | | | |
| Urine NAGL (ng/mL) | 411.0 ± 46.1 | 655.5 ± 96.1^{†} | 615.0 ± 177.8^{†} |
| Urine Creatinine | 1.1 ± 0.1 | 1.6 ± 0.2^{†} | 1.6 ± 0.1^{†} |
| (mg/dL) | | | |

^{†}vs sham,
‡ vs R-State,
& vs T-State

Plasma BUN, urine NAGL, and urine creatinine levels were significantly higher following a hypovolemic infusion of R-State and T-State TgHb compared to sham.

Kidney inflammatory markers were quantified in the form of a variety of interleukin (IL) and chemokine (C—X—C motif) ligand 1 (CXCL1) markers renal IL-6, renal IL-1, renal CXCL1 and renal IL-10. More specifically, upon completion of the experiment A) renal IL-6, B) renal IL-1, C) renal CXCL1, and D) renal IL-10 were measured to evaluate kidney inflammation.

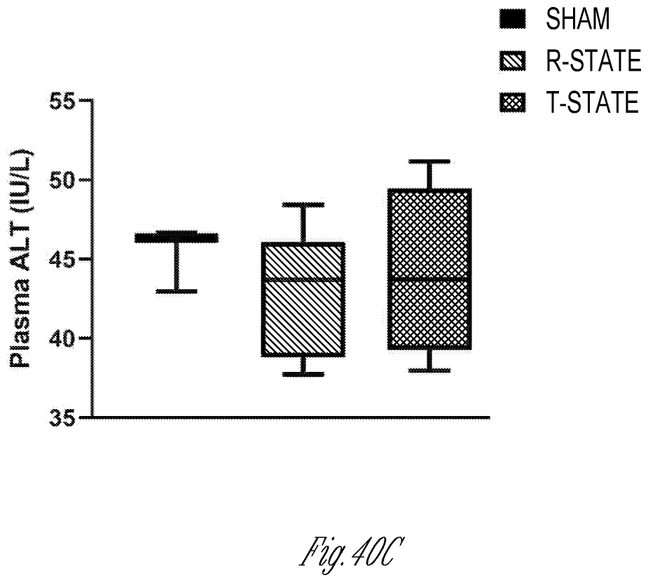

Upon completion of the experiment the Golden Syrian Hamsters were sacrificed where the kidney was collected. FIG. 39 portrays A) renal IL-6 B) renal IL-1, C) renal CXCL1 and D) renal IL-10 to evaluate kidney inflammation. Three Golden Syrian Hamsters were sacrificed without undergoing any protocol to establish a control.

Table 30 indicates levels of renal IL-6, renal IL-1, renal CXCL1 and renal IL-10 proteins that can indicate one or more states of kidney inflammation. Three Golden Syrian Hamsters were sacrificed without undergoing any protocol to establish a control. Significant to sham (†), significant to R-State (1), and significant to T-State (&).

TABLE 30
20 Minutes After Hypovolemic Infusion

| | Sham | R-State | T-State |
|---|---|---|---|
| Renal IL-6 (μg/mL) | 125.5 ± 6.1 | 230.9 ± 7.8^{†} | 258.7 ± 12.8^{†} |
| Renal IL-1 (μg/mL) | 131.4 ± 9.7 | 161.4 ± 12.3 | 175.3 ± 5.4 |
| Renal CLCX1 (μg/mL) | 163.4 ± 19.8 | 172.9 ± 15.6 | 183.4 ± 24.9 |
| Renal IL-10 (μg/mL) | 113.5 ± 20.0 | 186.7 ± 17.7^{†} | 169.4 ± 31.3 |

^{†}ys sham,
‡ vs R-State,
& vs T-State

Renal IL-6 levels after the hypovolemic infusion of R-State or T-Sate TgHb was significantly higher compared to sham. Renal IL-1 and renal CXCL1 had no significance compared to baseline or between groups of hypovolemic infusions of R-State or T-State TgHb. Renal IL-10 after hypovolemic infusions with R-State TgHb is significantly higher than sham.

### Liver Health

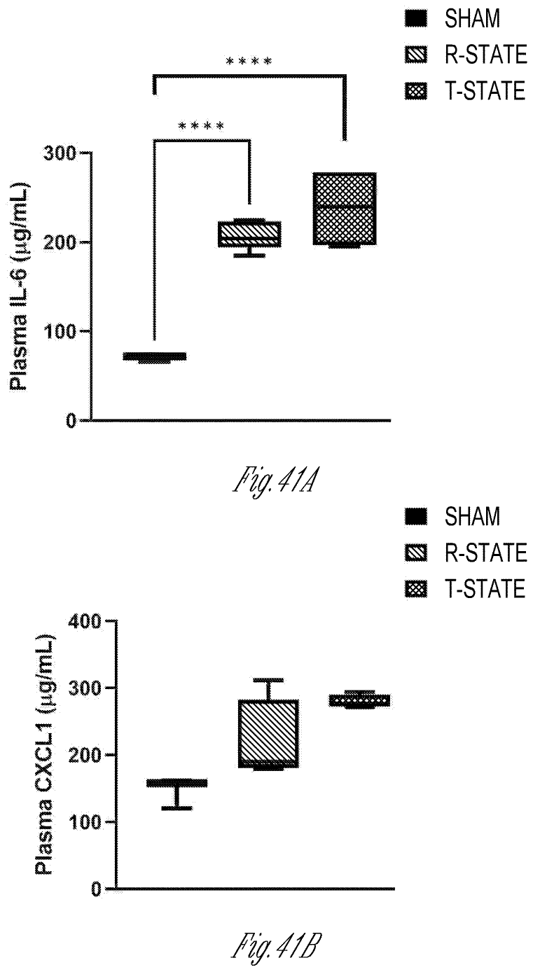

Upon completion of the experiment A) Hepatic CXCL1, B) plasma aspartate transaminase (AST), and C) plasma alanine transaminase (ALT) was measured to evaluate liver function and inflammation of the liver which is represented on FIG. 40.

Table 31 indicates levels of hepatic CXCL1, plasma AST and plasma ALT proteins that can indicate one or more status of the liver inflammation and liver function. Three Golden Syrian Hamsters were sacrificed without undergoing any protocol to establish a control. Significant to sham (†), significant to R-State (1), and significant to T-State (&).

TABLE 31
20 Minutes After Hypovolemic Infusion

| | Sham | R-State | T-State |
|---|---|---|---|
| Hepatic CXCL1 (μg/mL) | 116.1 ± 9.1 | 197.4 ± 31.9^{†} | 209.2 ± 20.5^{†} |
| Plasma AST (IU/L) | 31.6 ± 3.2 | 44.7 ± 1.0^{†} | 43.0 ± 1.0^{†} |
| Plasma ALT (IU/L) | 45.3 ± 2.1 | 42.7 ± 4.1 | 44.2 ± 5.4 |

^{†}vs sham,
‡ vs R-State,
& vs T-State

Hepatic CXCL1 and plasma AST levels were significantly higher after the hypovolemic infusion of R-State and T-State TgHb compared to sham. Plasma ALT levels were not significant to sham or after the hypovolemic infusion of R-State or T-State TgHb.

### Vasculature Health

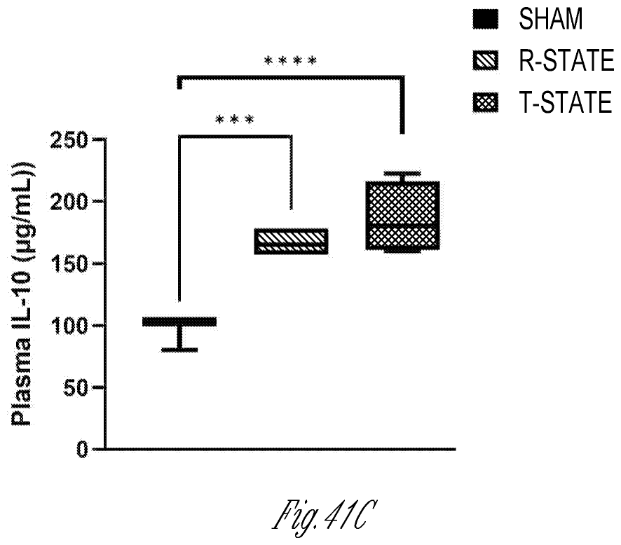

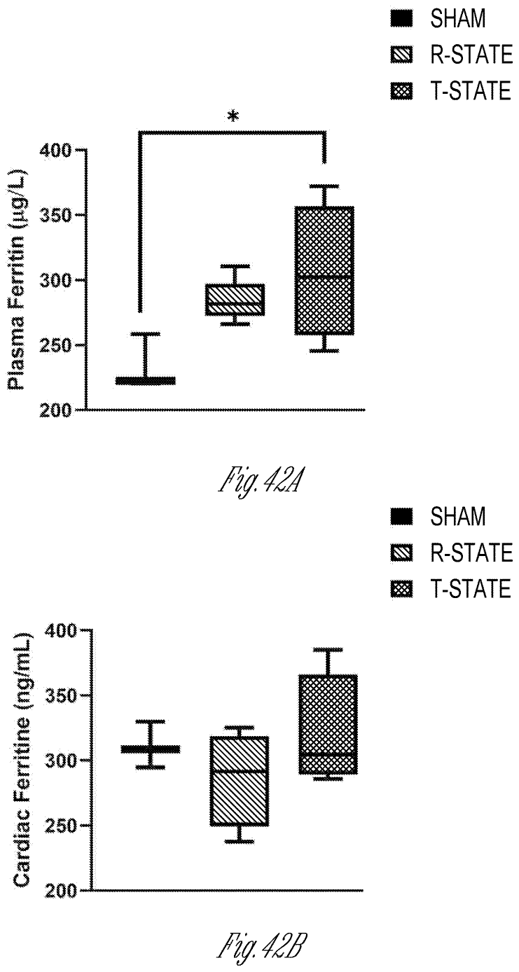

Following the conclusion of the experiment plasma was collected, plasma IL-6, plasma CXCL1, and plasma IL-10 were quantified. FIG. 41 indicates A) plasma IL-6, B) plasma CXCL1, and C) plasma IL-10 which can be speculated to be associated with vascular health.

Table 32 indicates levels of plasma IL-6, plasma CXCL1, and plasma IL-10 proteins that can indicate one or more status of liver inflammation and liver function. Three Golden Syrian Hamsters were sacrificed without undergoing any protocol to establish a control. Significant to sham (†), significant to R-State ($), and significant to T-State (&).

TABLE 32
20 Minutes After Hypovolemic Infusion

| | Sham | R-State | T-State |
|---|---|---|---|
| Plasma IL-6 (μg/mL) | 69.9 ± 3.6 | 207.9 ± 16.1^{†} | 238.1 ± 46.4^{†} |
| Plasma CLCX1 (μg/mL) | 146.9 ± 22.9 | 218.0 ± 62.8 | 279.7 ± 9.8 |
| Plasma IL-10 (μg/mL) | 96.2 ± 13.6 | 167.3 ± 10.6^{†} | 186.0 ± 29.8^{†} |

^{†}vs sham,
‡ vs R-State,
& vs T-State

Plasma IL-6 and plasma IL-10 levels were significantly higher compared to sham following a hypovolemic infusion of R-State or T-State TgHb. Plasma CXCL1 levels have no significance to baseline or between groups following a hypovolemic infusion of R-State or T-State TgHb.

### Ferritin and Bilirubin Concentration

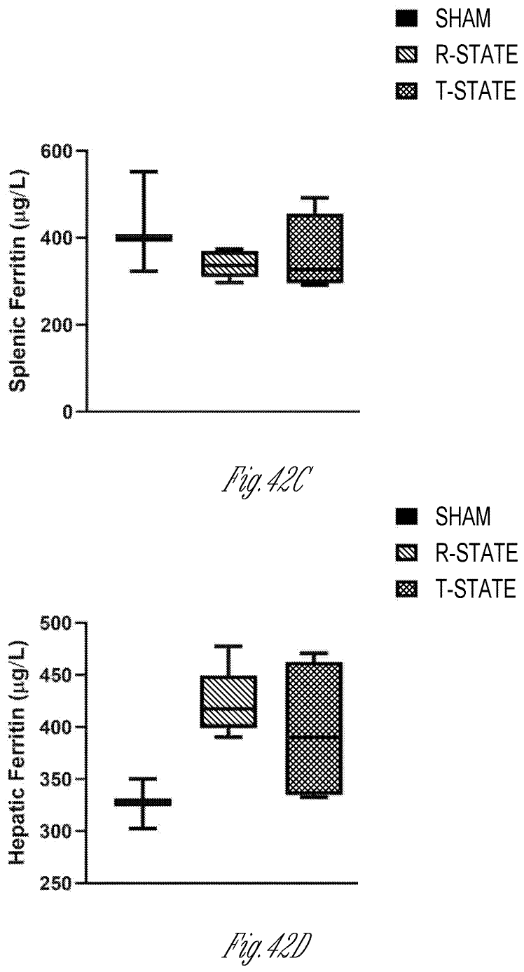

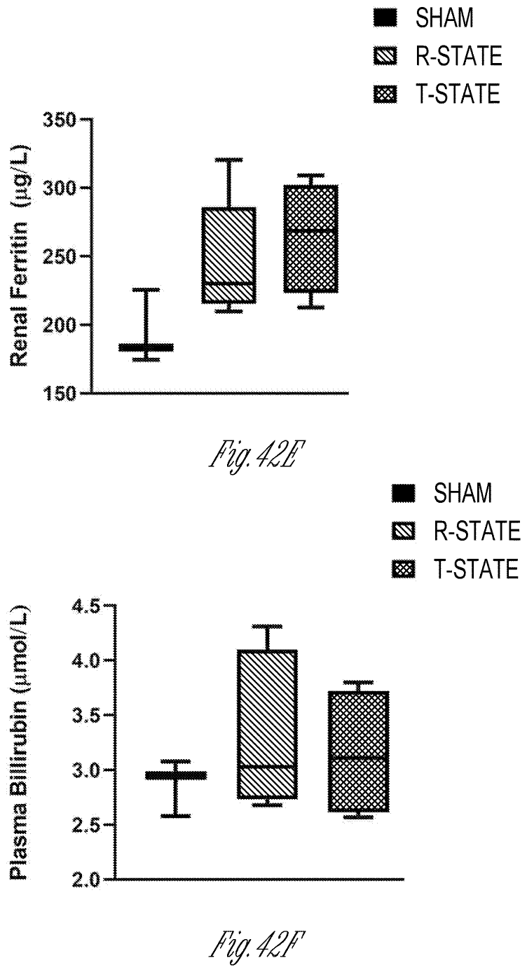

Following the conclusion of the experiment the ferritin and bilirubin concentration was quantified in the plasma, heart, spleen, liver, kidney and the ferritin concentration was quantified. FIG. 42 indicates A) plasma ferritin, B) cardiac ferritin, C) splenic ferritin D) hepatic ferritin, E) renal ferritin, F) plasma bilirubin which can be speculated to be associated with levels of iron in the circulation.

Table 33 indicates levels of plasma ferritin, cardiac ferritin, splenic ferritin, hepatic ferritin, renal ferritin, and plasma bilirubin proteins that can indicate iron concentration and levels of hemolysis in the vasculature and vital organs. Also, three Golden Syrian Hamsters were sacrificed without undergoing any protocol to establish a control. Significant to sham (†), significant to R-State (‡), and significant to T-State (&).

TABLE 33
20 Minutes After Hypovolemic Infusion

| | Sham | R-State | T-State |
|---|---|---|---|
| Plasma Ferritin (μg/L) | 233.9 ± 21.3 | 284.3 ± 16.2 | 305.7 ± 52.2^{†} |
| Cardiac Ferritin (ng/L) | 311.1 ± 17.7 | 286.7 ± 36.8 | 319.9 ± 44.5 |
| Splenic Ferritin (μg/L) | 425.4 ± 116.3 | 339.2 ± 30.7 | 360.1 ± 90.8 |
| Hepatic Ferritin (μg/L) | 326.8 ± 23.9 | 422.9 ± 32.9 | 395.9 ± 68.9 |
| Renal Ferritin (μg/L) | 194.7 ± 27.3 | 246.6 ± 44.0 | 264.7 ± 41.0 |
| Plasma Bilirubin | 2.9 ± 0.3 | 3.3 ± 1.0 | 3.1 ± 1.0 |
| (μmol/L) | | | |

^{†}vs sham,
‡ vs R-State,
& vs T-State

Plasma ferritin levels following a hypovolemic infusion of T-State TgHb was significantly higher compared to sham. Cardiac ferritin, splenic ferritin, hepatic ferritin, renal ferritin, and plasma bilirubin levels were not significantly different to sham or between groups following hypovolemic infusion of R-State or T-State TgHb.

### Example 10: Catecholamines

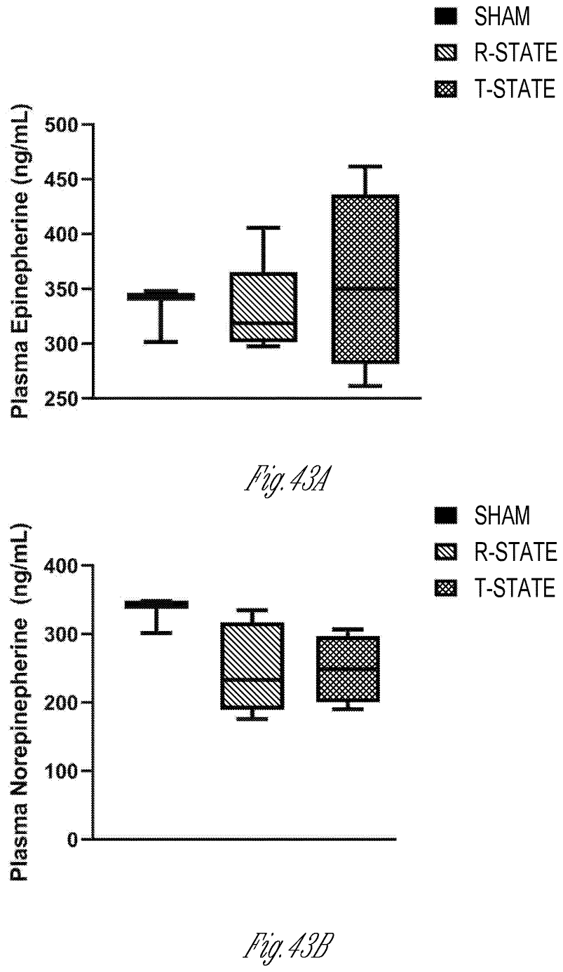

Following the conclusion of the experiment the plasma epinephrine and plasma norepinephrine concentration was quantified in the plasma. FIG. 43 indicates A) plasma epinephrine and B) plasma norepinephrine which can be speculated to be associated with levels of the nervous system.

Table 34 indicates levels of plasma epinephrine and plasma norepinephrine proteins that can indicate iron concentration and levels of nervous system reactions. Three Golden Syrian Hamsters were sacrificed without undergoing any protocol to establish a control. Significant to sham (†), significant to R-State (*), and significant to T-State (&).

TABLE 34
20 Minutes After Hypovolemic Infusion

| | Sham | R-State | T-State |
|---|---|---|---|
| Plasma Epinephrine (ng/mL) | 330.8 ± 25.4 | 330.6 ± 43.5 | 356.0 ± 82.3 |
| Plasma Norepinephrine | 330.8 ± 25.4 | 249.2 ± 66.4 | 248.8 ± 50.2 |
| (ng/mL) | | | |

† vs sham,
‡ vs R-State,
& vs T-State

Plasma epinephrine and plasma norepinephrine had no significance against sham or between groups after a hypovolemic infusion of R-State or T-State TgHb.

### Analysis

The results suggest that TgHb is a promising HBOC design. TgHb was tested against HBOC-201, a commercially available HBOC, and stroma free human hemoglobin. HBOC-201 and hemoglobin showed a significant amount of vasoconstriction and reduced flow in all sizes of arterioles, but TgHb had a more favorable outcome. The functional capillary density showed a fair drop in all three groups compared to baseline. Finally, all three groups showed acute inflammation and loss in function for the heart, liver, spleen, and kidney compared to the sham. However, TgHb did have noticeably lower concentrations of the pro-inflammatory markers and better organ function compared to HBOC-201 and hemoglobin.

In conclusion, TG can be used as means to bioconjugate hemoglobin for a more natural solution compared to the traditional use of glutaraldehyde. However, TgHb polymerization will need further adjustments to properly adjust the size and prevent any vascular dysfunction or inflammation responses.

The evolution of artificial blood continues to innovate on the ability to address common global challenges such as blood shortages or severe blood losses. TgHb proposes another type of hemoglobin-based oxygen carrier (HBOC). To be able to properly evaluate the usefulness of this HBOC, a key place like the microcirculation must be observed. The microcirculation addresses oxygen transport to the tissues and organs.

The microcirculation is broken up into the arterioles, venules, and capillaries. The arterioles are high-pressure and high-flow vessels where oxygen transport occurs. (Munoz, C. J.; Lucas, A.; Williams, A. T.; Cabrales, P. A Review on Microvascular Hemodynamics: The Control of Blood Flow Distribution and Tissue Oxygenation. Crit. Care Clin. 2020, 36 (2), 293-305. https://doi.org/10.1016/j.ccc.2019.12.011.) Understanding oxygen transport is to understand that convection drives oxygen delivery, thus larger diameter, and higher flow arterioles offload the most oxygen in the tissue. (Dewhirst, M. W. Concepts of Oxygen Transport at the Microcirculatory Level. In Seminars in radiation oncology; Elsevier, 1998; Vol. 8, pp 143-150; Farrell, K. J.; Witte, C. L.; Witte, M. H.; Mobley, W. P.; Kintner, K. Oxygen Exchange in the Mesenteric Microcirculation of the Dog. Am. J. Physiol.-Heart Circ. Physiol. 1979, 236 (6), H846-H853. https://doi.org/10.1152/ajpheart.1979.236.6.H846; Tsai, A. G.; Cabrales, P.; Intaglietta, M. Mechanisms of Oxygen Transport in the Microcirculation: Effects of Cell-Free Oxygen Carriers. In Blood Substitutes; Elsevier, 2006; pp 84-92.)

Furthermore, the varying sizes of arterioles create the myogenic response to properly regulate the flow (“oxygen transport”) to the tissues based on their needs. In such regard, when evaluating various HBOCs, the arterioles are of interest particularly to estimate the efficacy of HBOC-201, hHb, and TgHb. Although HBOC-201, hHb, and TgHb all showed levels of vasoconstriction, TgHb had the least amount of vasoconstriction in comparison to the other experimental groups regardless of their size. A common issue associated with HBOC is vasoconstriction due to hemoglobin toxicities created by nitric oxide scavenging. (Han, T. H.; Hyduke, D. R.; Vaughn, M. W.; Fukuto, J. M.; Liao, J. C. Nitric Oxide Reaction with Red Blood Cells and Hemoglobin under Heterogeneous Conditions. Proc. Natl. Acad. Sci. U.S.A 2002, 99 (11), 7763-7768. https://doi.org/10.1073/pnas. 122118299; Sharma, V. S.; Traylor, T. G.; Gardiner, R.; Mizukami, H. Reaction of Nitric Oxide with Heme Proteins and Model Compounds of Hemoglobin. Biochemistry 1987, 26 (13), 3837-3843. https://doi.org/10.1021/bi00387a015.)

hHb will oxidize into methemoglobin, methemoglobin will then dissociate into two hemoglobin dimers, alpha and beta, these dimers will then extravasate through fenestrated capillaries. The extravasation of the dimers into the tissue prevents the relaxation of the smooth muscles as it scavenges the nitric oxide. (Munoz, C. J., Lucas, A.; Williams, A. T.; Cabrales, P. A Review on Microvascular Hemodynamics: The Control of Blood Flow Distribution and Tissue Oxygenation. Crit. Care Clin. 2020, 36 (2), 293-305. https://doi.org/10.1016/j.ccc.2019.12.011.)

By hindering the vasoactivity of the arterioles, there becomes a lack of oxygen control to the tissue rendering it hypoxic. (Cabrales, P.; Tsai, A. G., Frangos, J. A.; Intaglietta, M. Role of Endothelial Nitric Oxide in Microvascular Oxygen Delivery and Consumption. Free Radic. Biol. Med. 2005, 39 (9), 1229-1237. https://doi.org/10.1016/j.freeradbiomed.2005.06.019.) Furthermore, the vasoconstriction causes an increase in mean arterial blood pressure and a decrease in heart rate as seen in the data for hHb, but not as drastic in HBOC-201 and TgHb. Lastly, due to the constriction seen in the arterioles, a drop in hydrostatic pressure inside the vessel also occurs. This drop in hydrostatic pressure results in the shunting or collapsing of functional capillaries. The capillaries are vessels with a diameter less than a red blood cell. A decrease in the number of functional capillaries generally suggest an increase in oxidative damage and death of the tissue. hHb presented the lowest number of functional capillaries followed by HBOC-201 and finally TgHb. This result is suggestive of TgHb's ability to perfuse the capillaries keeping the number of reactive oxygen species (ROS) to a minimum. These results associated with the microcirculatory health can suggest that TgHb structure is large and stable enough to prevent any sort of microvascular dysfunction associated with nitric oxide scavenging.

Furthermore, to explore the organ function, inflammation, and overall health associated with the infusion of HBOC-201, hHb, TgHb, the heart, liver, kidney and plasma were harvested. The markers quantified in the heart were cardiac CRP, IL-6, IL-1, TNF-α, MCP-1, and Troponin. Overall, the animals infused with hHb showed highest concentrations in all markers followed by HBOC-201 and then TgHb. A high concentration of cardiac CRP is related to issues in the carotid artery. (Sproston, N. R.; Ashworth, J. J. Role of C-Reactive Protein at Sites of Inflammation and Infection. Front. Immunol. 2018, 9, 754. https://doi.org/10.3389/fimmu.2018.00754.) High concentrations of IL-6 and IL-1 (pro-inflammatory cytokines) quantify the damage done to the myocardiocytes from oxidative stress. (Fontes, J. A.; Rose, N. R.; Čiháková, D. The Varying Faces of IL-6: From Cardiac Protection to Cardiac Failure. Cytokine 2015, 74 (1), 62-68. https://doi.org/10.1016/j.cyto.2014.12.024.) Increased TNF-α indicates alteration to calcium and nitric oxide utilization, the higher the concentration the potential of depressed cardiac function goes up. (Rolski, F.; Błyszczuk, P. Complexity of TNF-α Signaling in Heart Disease. J. Clin. Med. 2020, 9 (10). https://doi.org/10.3390/jcm9103267.) Increased MCP-1 establishes the severity of the injury done on the tissue as the higher the concentration a larger number of monocytes are recruited. (Niu, J.; Kolattukudy, P. E. Role of MCP-1 in Cardiovascular Disease: Molecular Mechanisms and Clinical Implications. Clin. Sci. Lond. Engl. 1979 2009, 117 (3), 95-109. https://doi.org/10.1042/CS20080581.) Troponin is a protein that modulates the contraction and relaxation, an increased concentration is related to an increase contractility and increase load on the heart. Regarding the kidney, plasma BUN, u-NGAL and Creatinine were quantified to speculate kidney function. All three markers are waste products found in the bloodstream; an increased concentration of these markers illustrates the lack of filtering in the kidney. Kidney inflammatory markers were quantified as well, renal IL-6, IL-1, CXCL1, and IL-10. As anticipated hHb saw the largest concentration of inflammatory markers. In the case of the heart, hHb proved to perform the worst, followed by HBOC-201 and TgHb with the best performance. Hepatic CXCL1, plasma AST, and plasma ALT were used to evaluate liver performance. Just as before, the data is suggestive that the animals infused with hHb performed the worst, followed by HBOC-201, and TgHb performed the best. Lastly, plasma IL-6, plasma CXCL1, and plasma IL-10 were quantified to associate inflammatory and immune responses in the vasculature due to oxidative damages. The animals infused with hHb saw the highest concentrations of all markers associating this with high amounts of oxidative damage to the vasculature, followed by HBOC-201, but TgHb showed the best results. The histology results all coincide with TgHb having superior performance compared to hHb and HBOC-201. It is hypothesized the organ issues seen with hHb and HBOC-201 is due to the instability of these molecules in the circulation resulting in the release of heme and iron. The heme moiety is commonly known for its interaction with TLR4 causing a cascaded of cytokines, adhesion molecules, and macrophage migration to vital organs. (Belcher, J. D.; Chen, C.; Nguyen, J.; Milbauer, L.; Abdulla, F.; Alayash, A. I.; Smith, A.; Nath, K. A.; Hebbel, R. P.; Vercellotti, G. M. Heme Triggers TLR4 Signaling Leading to Endothelial Cell Activation and Vaso-Occlusion in Murine Sickle Cell Disease. Blood 2014, 123 (3), 377-390. https://doi.org/10.1182/blood-2013-04-495887; Belcher, J. D.; Beckman, J. D.; Balla, G.; Balla, J.; Vercellotti, G. Heme Degradation and Vascular Injury. Antioxid. Redox Signal. 2010, 12 (2), 233-248. https://doi.org/10.1089/ars.2009.2822.)

The increase in iron concentration results in oxidative damage to the RBC themselves and the neighboring tissues resulting in higher amounts of hemolysis forcing a negative feedback loop. (Balla, J.; Vercellotti, G. M.; Jeney, V.; Yachie, A.; Varga, Z.; Jacob, H. S.; Eaton, J. W.; Balla, G. Heme, Heme Oxygenase, and Ferritin: How the Vascular Endothelium Survives (and Dies) in an Iron-Rich Environment. Antioxid. Redox Signal. 2007, 9 (12), 2119-2137. https://doi.org/10.1089/ars.2007.1787; Cappellini, M.-D.; Cohen, A.; Eleftheriou, A.; Piga, A.; Porter, J.; Taher, A. Iron Overload; Thalassaemia International Federation, 2008.)

This can be seen with the high concentrations of bilirubin in hHb and HBOC-201. To quantify iron concentration the levels of ferritin in the plasma and liver suggest that hHb and HBOC-201 have a high amount of iron in the circulation. Ferritin is an iron scavenging protein and is released from the tissue depending on the levels of hepcidin which is directly related to the levels of iron. (Collins, J. F.; Wessling-Resnick, M.; Knutson, M. D. Hepcidin Regulation of Iron Transport. J. Nutr. 2008, 138 (11), 2284-2288. https://doi.org/10.3945/jn.108.096347.)

Hemoglobin in the T-state favors pre-capillary O2 delivery to tissues, which can increase the O2 flux across the vessel wall, triggering vascular autoregulatory mechanisms to prevent O2 toxicity. (Winslow, R. M. Oxygen: The Poison Is in the Dose. Transfusion (Paris) 2013, 53 (2), 424-437. https://doi.org/10.1111/j. 1537-2995.2012.03774.x.) Oversupply of O2 by T-state Hb could also favor the formation of reactive oxygen species (ROS). On the other hand, R-state Hb tends to tightly bind O2 and mostly release O2 at the capillary level, where tissue pO2 is low and O2 is needed, thus preventing vascular hyperoxygenation and decreasing vasoconstriction and ROS production. (Tsai, A. G., Cabrales, P., Manjula, B. N., Acharya, S. A., Winslow, R. M., & Intaglietta, M. (2006). Dissociation of local nitric oxide concentration and vasoconstriction in the presence of cell-free hemoglobin oxygen carriers. Blood, 108 (10), 3603-3610. https://doi.org/10.1182/blood-2006-02-005272).

The creation of a polymerized T-State or R-State TgHb establishes new engineering tactics for HBOC design. This technique drastically affects rheological properties, as the R-State has a viscosity almost 8× that of its T-State counterpart. This high viscous solution (R-State) hinders the microcirculation as flow the vascular flow in the arterioles is significantly impaired. However, the functional capillary density outperforms the T-State TgHb.

The use of these two might prove to be problematic as the model of a hypovolemic infusion is done on an animal at hemostasis with its oxygen transport properties. This effect is seen in the inflammatory and functional markers being significantly affected in both cases R-State and T-State.

TgHb in situations of hemorrhagic shock requires the restoration of vascular homeostasis. This is done not just through volume replacement, but with a fluid with adequate biophysical properties.

biophysical properties. In hypovolemic shock the vascular tonality can be manipulated through shear stress on the walls of the endothelium. Shear stress is directly correlated with viscosity, higher the viscosity the greater the shear stress on the endothelium and as a resalt a release of nitric oxide a potent vasodilator to increase the flow rate in the microvasculature driving greater oxygen deliver to the tissues.

By controlling the degree of polymerization of hemoglobin you can control biophysical properties (half-life, visocisty, oxidative rate, and collod pressure). Furthermore, the state of hemoglobin prior to oxidation (R-State or T-State) will create additional changes to the biophysical properties.

The engineering of the hemoglobin solution (including the choice of solvent) with a variety of biophysical properties are dependent on the application. As hemoglobin will oxidize in time generating reactive oxidative species and methemoglobin as a result. The methemolobin concentration is believed to be of concern as it is prone for dimerization and release of hemoglobin dimers that will scavenge nitric oxide and home which will trigger tool like receptor 4 causing inflammation, heightened immune response, and a number of other adverse effects. Controlling the half-life and oxidative rate allows for the use of the hemoglobin polymer to offload oxygen to replenish any oxygen debt created through the pathophysiological event and limit the toxic events associated with hemoglobin circulating freely converting to methemoglobin.

Of note, the exemplary embodiments of the disclosure described herein do not limit the scope of the invention since these embodiments are merely examples of the embodiments of the invention. Any equivalent embodiments are intended to be within the scope of this invention. Indeed, various modifications of the disclosure, in addition to those shown and described herein, such as alternative useful combinations of the elements described, may become apparent to those skilled in the art from the description. Such modifications and embodiments are also intended to fall within the scope of the appended claims.

1. A method comprising:
introducing a transglutaminase to a solution that contains a plurality of hemoglobins to polymerize at least a portion of the plurality of the hemoglobins to form polymerized hemoglobins.

2. The method of claim 1, wherein, prior to introducing the transglutaminase, solid content of the plurality of hemoglobins in the solution is from about 1 g/dL to about 25 g/dL.

3. (canceled)

4. (canceled)

5. The method of claim 2, wherein introducing the transglutaminase comprises introducing the transglutaminase at a ratio of from about 2 to about 10 enzyme units of the transglutaminase per gram of the plurality of hemoglobins.

6. (canceled)

7. The method of claim 1, wherein the transglutaminase comprises a calcium-independent transglutaminase.

8. A composition comprising:
a plurality of hemoglobins, wherein at least a portion of the plurality of hemoglobins comprises polymerized hemoglobins; and
an amount of transglutaminase.

9. (canceled)

10. An aqueous solution comprising the composition of claim 8.

11. The aqueous solution of claim 10, wherein solid content of the plurality of hemoglobins in the solution is from about 1 g/dL to about 25 g/dL.

12. (canceled)

13. (canceled)

14. (canceled)

15. The aqueous solution of claim 11, wherein the aqueous solution exhibits a colloid osmotic pressure of less than about 50 mmHg.

16. (canceled)

17. (canceled)

18. The aqueous solution of claim 11, wherein solid content of the plurality of hemoglobins in the solution is about 10 g/dL.

19. The aqueous solution of claim 18, wherein, when an oxygenation state of the polymerized hemoglobins is about a fully oxygenated state of the polymerized hemoglobins, a viscosity of the aqueous solution at a shear rate of about 100 s⁻¹ at about 37° C. is from about 35 cP to about 50 cP.

20. (canceled)

21. (canceled)

22. The composition of claim 8, wherein the transglutaminase comprises a calcium-independent transglutaminase.

23. An aqueous solution comprising:
a plurality of hemoglobins, wherein at least a portion of the plurality of hemoglobins comprises transglutaminase-polymerized hemoglobins (TgHbs), and
wherein a viscosity of the aqueous solution at a shear rate of about 100 s⁻¹ at about 37° C. changes by about 25 cP or more when an oxygenation state of the TgHbs changes from about a deoxygenated state to about a fully oxygenated state.

24. The aqueous solution of claim 23, wherein the viscosity of the aqueous solution at the shear rate of about 100 s⁻¹ at about 37° C. changes by about 30 cP when the oxygenation state of the TgHbs changes from about deoxygenated state to about fully oxygenated state.

25. The aqueous solution of claim 23, wherein solid content of the plurality of hemoglobins in the solution is from about 1 gram per g/dL to about 25 g/dL.

26. (canceled)

27. (canceled)

28. The aqueous solution of claim 23, wherein solid content of the plurality of hemoglobins in the solution is from about 10 gram per deci-Liter (g/dL) to about 20 g/dL.

29. (canceled)

30. The aqueous solution of claim 28, wherein the aqueous solution exhibits a colloid osmotic pressure of less than about 50 mmHg.

31. (canceled)

32. The aqueous solution of claim 28, wherein the aqueous solution exhibits a colloid osmotic pressure of from about 30 mmHg to about 35 mmHg.

33. The aqueous solution of claim 28, wherein solid content of the plurality of hemoglobins in the solution is about 10 g/dL.

34. The aqueous solution of claim 33, wherein, when the oxygenation state is around the fully oxygenated state of the TgHbs, the viscosity of the aqueous solution at a shear rate of about 100 s⁻¹ at about 37° C. is from about 35 cP to about 50 cP.

35. The aqueous solution of claim 34, wherein, when the oxygenation state is around the fully oxygenated state of the TgHbs, the viscosity at the shear rate of about 100 s⁻¹ at about 37° C. is about 42 cP.
